(19) United States
(12) Patent Application Publication
(10) Pub. No.: US 2005/0064510 A1
(43) Pub. Date: Mar. 24, 2005

(54) Ligand analysis

(76) Inventors: Can D. Akyuz, New Brunswick, NJ (US); D. Allen Annis, Cambridge, MA (US); William H. Lee, Framingham, MA (US); Gergely M. Makara, Norwood, MA (US); Ciamac C. Moallemi, Stanford, CA (US); Huw M. Nash, Cambridge, MA (US); Zhongli Zheng, Lexington, MA (US)

Correspondence Address: FISH & RICHARDSON PC, 225 FRANKLIN ST, BOSTON, MA, 02110, US

(21) Appl. No.: 10/797,551

(22) Filed: Mar. 10, 2004

Related U.S. Application Data
(60) Provisional application No. 60/453,473, filed on Mar. 10, 2003.
(60) Provisional application No. 60/453,457, filed on Mar. 10, 2003.
(60) Provisional application No. 60/460,910, filed on Apr. 7, 2003.
(60) Provisional application No. 60/463,025, filed on Apr. 15, 2003.
(60) Provisional application No. 60/502,670, filed on Sep. 12, 2003.

(51) Int. Cl. G01N 33/53

(52) U.S. Cl. 435/7.1

(57) ABSTRACT

Methods for the analysis of receptor-ligand pairs are herein described. The methods can provide information about the binding affinity of a ligand to a receptor or can provide information about the binding kinetics of a ligand to a receptor. In some instances, the methods provide a very general system for evaluating and/or optimizing receptor-ligand interactions, which can be applied to a variety of types of receptor-ligand interactions, for example protein-small molecule interactions.

### CROSS-REFERENCE TO RELATED APPLICATIONS

This application claims priority to U.S. Provisional Application Ser. No. 60/453,473, filed on Mar. 10, 2003, U.S. Provisional Application Ser. No. 60/453,457, filed on Mar. 10, 2003, U.S. Provisional Application Ser. No. 60/460,910, filed on Apr. 7, 2003, U.S. Provisional Application Ser. No. 60/463,025, filed on Apr. 15, 2003, and U.S. Provisional Application Ser. No. 60/502,670, filed on Sep. 12, 2003; each of which is incorporated herein by reference in its entirety.

### BACKGROUND

The effort to discover drugs to treat various diseases has been revolutionized with technologies such as combinatorial chemistry and elucidation of the human genome. High throughput technologies in both genomics and biological screening, together with enormous advances in computer technology have made it more feasible to use these tools as part of rational approaches in the search for new medicines.

However, realizing the full potential of chemical genomics requires the development of generic and efficient tools that apply the strengths of technologies such as combinatorial chemistry to the progression of lead compounds for newly discovered disease targets. General techniques to directly assess binding mechanisms and evaluate ligand affinities in a mixture format are currently lacking, despite advances in chemical synthesis that have enabled considerable sophistication in the construction of diverse compound libraries to probe protein function. Genome and proteome analyses are rapidly increasing the number of human and bacterial proteins identified as targets for small molecule therapy of human disease, creating a critical need for methods of evaluating protein-ligand binding that are applicable to novel targets for which functional assays are unavailable. Affinity ranking and classification of ligand chemotypes according to binding site are especially desirable for targets where X-ray co-crystal structures are inaccessible for the rationalization of ligand structure-activity relationships.

One early predictor of a viable drug candidate is its K_{d}, equilibrium dissociation constant with its target biomolecule. K_{d} is a measure of how tightly the drug candidate binds to its target, and is a predictor with regard to the candidate's efficacy, potential toxicity, and side effects. The kinetics of a drug candidate, e.g., the dissociation rate, k₂, of a drug candidate from its target, can also be an early predictor of whether the drug candidate will ultimately become a drug. k₂ is a measure of how tightly the drug candidate binds to its target, and can be an indicator of the candidate's potential efficacy, toxicity and side effects. The ability to estimate various binding properties, including K_{d} and k₂, in an efficient and streamlined manner could significantly improve the drug discovery process.

### SUMMARY

Affinity selection-mass spectrometry (AS-MS) techniques hold particular promise for the evaluation of binding properties of ligands (e.g., small molecules and organic compounds) to receptors (e.g., proteins and enzymes). These techniques uniquely identify protein-bound components from complex mixtures by virtue of their molecular weights or collision-induced fragmentation patterns, making possible the simultaneous evaluation of multiple ligands from compound libraries. The exquisite sensitivity of modern MS technology enables AS-MS experiments using only minimal amounts of a purified biomolecular receptor. In contrast to cellular or other complex biochemical assays, AS-MS techniques report only compounds that bind selectively to the target of interest, thus precluding false positives arising from off-target activity.

We describe here a multidimensional chromatography-mass spectrometry method for simultaneously ranking the affinity of multiple ligands for a protein receptor while in some instances also demonstrating whether the ligands bind the same site as select competitor ligands or through interaction at an allosteric binding site. In instances where two ligands bind at different sites, the methods can in some instances yield their absolute binding affinity and a quantitative assessment of the degree of binding cooperativity between them.

In one aspect, the invention features a method of analyzing a binding affinity of a receptor for a ligand in a plurality of mixtures including a receptor, E, a ligand, Sᵢ, and a receptor-ligand binding pair, ESᵢ. The method includes the following steps:
(a) providing a plurality of mixtures, each mixture including a receptor, [E]₀, a ligand [Sᵢ]₀, and a titrant, T, wherein the concentration of one or more of E, Sᵢ, T are chosen such that the relative ability of T to displace S can be determined;
(b) allowing each of the plurality of mixtures to achieve equilibrium;
(c) separating the receptor-ligand binding pairs, ESᵢ, from the unbound ligands, Sᵢ, for each of the plurality of mixtures;
(d) determining the signal response from an analytical device for the receptor-ligand binding pair, ESᵢ in each of the plurality of mixtures; and
(e) evaluating the signal responses from step (d) of the receptor-ligand binding pairs, ESᵢ, to determine binding affinity of the ligand Sᵢ to the receptor E.

In some instances, each mixture is selected such that the concentration of T relative to [E]₀ and [Sᵢ]₀, is chosen to allow for comparison of the relative ability of Sᵢ to displace T.

In some instances, the method includes providing a plurality of mixtures, each mixture including a initial concentration of receptor, [E]₀, an initial concentration of ligand [Sᵢ]₀, and a known concentration of a titrant wherein [E]₀ and [Sᵢ]₀ are constant throughout each of the plurality of mixtures, the [Sᵢ]₀ is approximately the same within each of the plurality of mixtures, and the concentration of the titrant is varied within the plurality of mixtures.

In some instances, the plurality of mixture each includes a plurality of ligands Sᵢ, and a plurality of receptor-ligand binding pairs, ESᵢ, and the signal response is determined for at least two of the receptor-ligand binding pairs, ESᵢ, and the relative binding of the receptor-ligand binding pairs, ESᵢ, is determined.

In some instance, for example, when the mixture includes a plurality of ligands, at least about 90% of the plurality of ligands, Sᵢ, have a unique molecular mass.

In some instances, each mixture is selected such that the concentration of T relative to [E]₀ and [Sᵢ]₀, is chosen such that the binding affinity of a first ligand, S₁, can be compared with the binding affinity of a second ligand, S₂, to provide a measure of the relative binding affinity of S₁ for E and S₂ for E.

In some instances the binding affinities are relative binding equilibrium constants, K_{di}S.

In some instances, the method includes calculating the ACE₅₀ of one or more ligands, which is the titrant concentration at which the signal response of a receptor-ligand pair reaches 50% of its value when the titrant concentration is 0.

In some instances, relative K_{d}s of a plurality of ligands are determined such that the ligand with the lowest ACE₅₀ value has the highest K_{d} of the mixture of ligands, and the ligand with the highest ACE₅₀ value has the lowest K_{d} of the mixture of ligands.

In some instances, the method includes calculating the K_{di} of a receptor-ligand binding pair, ESᵢ, in the plurality of mixtures by fitting the change in concentration of the receptor-ligand binding pairs, [ESᵢ], in each of the plurality of mixtures as a function of the titrant concentration to the equation of formula (I) or an equation derived from formula (I)

$${K}_{d i} = \frac{( {[ E ]}_{0} - {\sum }_{i} [ E {S}_{i} ] ) ( {[ {S}_{i} ]}_{0} - [ E {S}_{i} ] )}{[ E {S}_{i} ]} . \qquad formula ( I )$$

In some instances, the relative K_{di}s of a plurality of ligands Sᵢ are determined.

In some instances, the initial concentration of receptor, [E]₀, is known and the initial concentration of the ligand, [Sᵢ]₀ is known.

In some instances, the concentration of the receptor, [E]₀, is greater than the sum total of the concentration of the ligands, [Sᵢ]₀.

In some instances, the method also includes determining the whether a ligand Sᵢ binds to the receptor E bind in a competitive manner, an allosteric manner, or a non-competitive manner. For example, if a receptor ligand-pair ESᵢ maintains a relatively constant signal response in each of the plurality of mixtures the ligand Sᵢ binds to the receptor E in a non-competitive manner. In some instances, the method includes determining the variation in the ratio of signal response of a receptor ligand pair ESᵢ to response of the receptor-titrant pair versus the concentration of the titrant for each of the plurality of mixtures, wherein if the ratios for each of the plurality of mixtures have a linear relationship with the titrant concentration, then the ligand Sᵢ binds to the receptor in a competitive manner, and wherein if the ratios for each of the plurality of mixtures have a non-linear relationship, than the ligand Sᵢ binds to the receptor in an allosteric manner.

In some instances, the receptor is a biomolecule, a polypeptide, an enzyme, or a nucleic acid.

In some instances, the ligand is an organic molecule, such as a pharmaceutical compound or a small molecule (e.g., a molecule having a molecular weight of less than about 600 a.m.u.), or a polypeptide.

In some instances, the plurality of mixtures achieves equilibrium of receptor-ligand binding pair, ESᵢ, unbound receptor, and unbound ligand.

In some instances, the method includes using liquid chromatography, for example, reverse-phase liquid chromatography, which can be used under conditions, which dissociate a receptor-ligand binding pair.

In some instances, the receptor-bound ligand is separated from each of the plurality of mixtures using size-exclusion-chromatography or ultrafiltration.

In some instances, the signal response is determined using mass spectrometry.

In some instances, the method includes disrupting the receptor-ligand binding pair, ESᵢ.

In some instances, the signal response of a receptor-ligand binding pair, ESᵢ, is determined by measuring the relative amount of ligand, Sᵢ, in the receptor-ligand binding pair, ESᵢ, in each of the plurality of mixtures.

In some instances, the relative amount of ligand, Sᵢ, is determined by evaluating a signal response from a mass spectrometer.

In another aspect, the invention features a method for determining the equilibrium dissociation constant, K_{d}, of a receptor-ligand binding pair. The method includes the following steps:
(a) providing a mass spectrometer calibrated to the ligand of the receptor-ligand binding pair;
(b) providing a plurality of mixtures, each mixture including a receptor, [E]₀, and a ligand, [S]₀, wherein the concentration of one or more of E₀, and S₀ is chosen such that the binding affinity of S to E can be determined;
(c) allowing each of the plurality of mixtures to reach equilibrium of bound receptor-ligand binding pairs, ES, unbound receptor, and unbound ligand;
(d) separating the receptor-bound ligand from each of the plurality of mixtures;
(e) determining the signal response from the mass spectrometer for the receptor-ligand binding pairs in each of the plurality of mixtures; and
(f) using information known, measured or acquired in steps a-e to fit the concentration of receptor-ligand pair, [ES], and initial, known ligand concentration, [S]₀, to the equation of formula (I)

$${K}_{d} = \frac{( {[ E ]}_{0} - [ E S ] ) ( {[ S ]}_{0} - [ E S ] )}{[ E S ]} \qquad formula ( I )$$

for each of the plurality of mixtures, yielding the K_{d} of the receptor-ligand binding pair.

In some instances, each of the plurality of mixtures includes an initial concentration of receptor, [E]₀, and an initial, known concentration of ligand, [S]₀, wherein [E]₀ is about the same in each of the plurality of mixtures and [S]₀ is varied in each of the plurality of mixtures.

In some instances, the method also includes determining the initial receptor concentration [E]₀ in the mixtures.

In some instances, the receptor is a biomolecule, a polypeptide, an enzyme, or a nucleic acid.

In some instances, the ligand is an organic molecule, such as a pharmaceutical compound or a small molecule (e.g., a molecule having a molecular weight of less than about 600 a.m.u.), or a polypeptide.

In some instances, the plurality of mixtures reach equilibrium of bound receptor-ligand binding pairs, unbound receptor, and unbound ligand.

In some instances, the receptor-bound ligands are separated from the mixture using size-exclusion-chromatography.

In some instances, the method includes using liquid chromatography.

In some instances, the method also includes disrupting the receptor-ligand binding pairs, ES, for example by using reverse-phase liquid chromatography under high temperature conditions.

In some instances, the concentration of the receptor-ligand binding pair, [ES], is determined by measuring the amount of ligand in the receptor-ligand binding pairs, ES, in each of the plurality of mixtures.

In another aspect, the invention features a method of analyzing the binding kinetics of a receptor-ligand binding pair. The method includes the following steps:
(a) providing a mixture including a receptor, [E]₀, and a ligand, [Sᵢ]₀;
(b) allowing the mixture to reach equilibrium of receptor, [E], ligand, [Sᵢ], and receptor-ligand binding pair, [ESᵢ];
(c) treating the mixture with an excess of a competitive inhibitor, I;
(d) measuring a decrease in the receptor-ligand binding pair at a plurality of time points by;
   (i) separating the receptor-ligand binding pair from the unbound ligand; and
   (ii) determining a signal response of the receptor-ligand binding pair for each of the plurality of time points with an analytical device; and
(e) using the information known, measured, or acquired from steps (a)-(d) to evaluate the binding kinetics of the receptor-ligand binding pair.

In some instances, the signal response of the receptor-ligand binding pair is measured with an analytical device.

In some instances, the mixture includes a plurality of ligands, Sᵢ.

In some instances, for example, when the mixture includes a plurality of ligands, Sᵢ, at least 90% of the plurality of ligands, Sᵢ, have a unique molecular mass.

In some instances, the binding kinetics are evaluated using the information known, measured, or acquired from to calculate the dissociation rate, kₛ₂ of the receptor-ligand binding pair by fitting the change in signal response of the receptor-ligand binding pair over time to the equation of formula (XVIII) or a derivative thereof
[ES]=[ES]ₜ₌₀e^{-ks2·t} formula (XVIII).

In some instances, the method includes identifying a ligand that binds in a non-competitive manner wherein if the a ligand-receptor binding pair maintains a relatively constant concentration at each of the plurality of time points, than the ligand is binding to the receptor in a non-competitive manner.

In some instances, the binding kinetics of at least two of the plurality of ligands, Sᵢ, are compared.

In some instances, the receptor is a biomolecule, a polypeptide, an enzyme, or a nucleic acid.

In some instances, the ligand and/or competitive inhibitor is an organic molecule, such as a pharmaceutical compound or a small molecule (e.g., a molecule having a molecular weight of less than about 600 a.m.u.), or a polypeptide.

In some instances, the method includes subjecting the receptor-bound ligand to liquid chromatography.

In some instances, the receptor-bound ligand is separated from the unbound ligand using size-exclusion-chromatography.

In some instances, the signal response is determined using mass spectrometry.

In some instances, the method also includes disrupting the receptor-ligand binding pair.

In some instances, the signal response of the receptor-ligand binding pair is determined by measuring the relative amount of ligand in the receptor-ligand binding pair.

In some instances, the method also includes determining the half-life, t_{1/2}, of the receptor ligand binding pair.

In one aspect, the invention features a method of identifying a receptor-ligand complex from a sample. The method includes the following steps:
(a) providing a sample including a receptor-ligand complex;
(b) subjecting the sample to size-exclusion-chromatography, wherein an eluant from the size-exclusion-chromatography is passed through a UV detector and a multiport valve including a sample loop;
(c) detecting the receptor-ligand complex from the sample using the UV detector, wherein detection of the receptor-ligand complex activates a controller that is in communication with the UV detector and the multiport valve including a sample loop, wherein the controller is calibrated to activate the multiport valve at a time when the receptor-ligand complex is present in the sample loop, and wherein activation of the multiport valve transfers the receptor-ligand complex present in the sample loop to a chromatographic device; and
(d) identifying a ligand from the receptor-ligand complex, thereby identifying the receptor-ligand complex.

In some instances, sample also includes an unbound ligand.

In some instances, the receptor-ligand complex is separated from unbound ligand.

In some instances, the method also includes dissociating the receptor-ligand complex.

In some instances, the dissociation of the receptor-ligand complex occurs in the chromatographic device.

The receptor of the receptor-ligand complex can be, for example, a polypeptide, an enzyme, or a nucleic acid.

The ligand of the receptor-ligand complex can be, for example, a polypeptide, an organic molecule, or a nucleic acid.

In some instances, the controller is calibrated manually. In other instances, the controller is calibrated using a computer software program.

In some instances, the chromatographic device is a reverse-phase column.

In some instances, the dissociated ligand is identified using mass spectroscopy.

In some instances, the ligand is devoid of an identifying tag. In some instances, the ligand has an identifying tag, which can be, for example, a fluorescent tag or a radioactive tag.

In another aspect, the invention features a method of identifying a plurality of receptor-ligand complexes from a sample. The method includes:
(a) providing a sample including a plurality of receptor-ligand complexes;
(b) subjecting the sample to size-exclusion-chromatography, wherein an eluant from the size-exclusion-chromatography is passed through a UV detector and a multiport valve including a sample loop;
(c) detecting the receptor-ligand complexes from the sample using the UV detector, wherein detection of the receptor-ligand complexes activates a controller that is in communication with the UV detector and the multiport valve including a sample loop, wherein the controller is calibrated to activate the multiport valve at the time when at least a portion of the receptor-ligand complexes are present in the sample loop, wherein activation of the multiport valve transfers the receptor-ligand complexes present in the sample loop to a chromatographic device; and
(d) identifying a dissociated ligand from a dissociated receptor-ligand complex.

In some instances, the sample also includes a plurality of unbound ligands.

In some instances, the receptor-ligand complexes are separated from unbound ligand.

In some instances, the method includes identifying a plurality of dissociated ligands.

In some instances, the method includes a single receptor.

In some instances, the method also includes dissociating the receptor-ligand complexes.

In some instances, the dissociation of the receptor-ligand complexes occurs in the chromatographic device.

The receptor of the receptor-ligand complex can be, for example, a polypeptide, an enzyme, or a nucleic acid. The ligand of the receptor ligand complex can be, for example, a polypeptide, an organic molecule, or a nucleic acid.

In some instances, the controller is calibrated manually. In other instances, the controller is calibrated using a computer software program.

In some instances, the chromatographic device is reverse-phase column.

In some instances, one or more of the dissociated ligands are identified using mass spectroscopy.

In some instances, the ligands are devoid of an identifying tag. In some instances, the ligand has an identifying tag, which can be, for example, a fluorescent tag or a radioactive tag.

In some instances, at least about 90% of the plurality of ligands have a unique molecular mass. For example, about 90, 91, 92, 93, 94, 95, 96, 97, 98, 99, or 100% of the ligands can have a unique molecular mass.

In some instances, the invention relates to a process for identifying one or more chemical compounds having desirable binding characteristics. In general, such compounds can be identified using the following steps: (1) providing mixtures of ligands as structural variants (i.e., related compounds) of the chemical structure of a first ligand, (in some instances, these compounds are uniquely identifiable by their molecular mass), (2) analyzing the mixture structural variants to qualitatively and/or quantitatively rank the affinities of the structural variants relative to the affinity of the original ligand for its biomolecule receptor or the binding kinetics of the original ligand for its biomolecule receptor, and (3) using the results to identify desirable ligands and to design related ligands for subsequent cycles of mixture, synthesis, and/or ranking of ligands.

Any of the methods described herein can be used in an iterative or repetetive manner, or in a sequential manner, for example with another method described herein. For example, if a first method is used to provide binding information (e.g., relative binding affinity) about a ligand or a mixture of ligands, additional ligands can be designed or tested using the information obtained in the first method. The additional ligands can be further evaluated, for example using the first method, or alternatively, using a second method described herein. For example, a mixture of ligands can be first evaluated by rank of binding affinities; the binding affinity information can then be used to design additional ligands, which are subsequently ranked by binding affinity or binding kinetics.

While the results of one or more the methods described herein can be used to direct the synthesis or assembly of additional ligands for evaluation, in some examples a single ligand or mixture of ligands can be evaluated using multiple methods or using a single method where one or more steps of the method are re-run. For example, a single ligand or mixture of ligands can be evaluated by assessing the binding affinity (e.g., K_{d}) and also assessing the binding kinetics (e.g., k_{off}). These methods for assessing binding affinity and binding kinetics can be performed in any order and can also be performed in an iterative manner. In some instance, a single method is run with the same ligand or mixture of ligands to ensure reproducibility of the method.

The methods described herein can provide a very efficient method for the identification of a receptor-ligand complex in a sample. These methods can directly deliver the receptor-ligand complex from a size-exclusion chromatography stage to an analytical device, such as an LC-MS device, which can allow for rapid sample screening as well as reduction of sample loss due to the use of a fraction collector or the adhesion of the sample to the surface of the analytical device. Additionally, the desired output of the methods can be achieved with mixtures of ligands (e.g., combinatorial libraries) as opposed to single ligands, and does not require purification of the ligands. In some instances, the methods described herein have the advantage of providing the ability to run samples on a microscale. Another advantage of can include providing a high-throughput method that can be run with automation. Accordingly, a large number of compounds can be screened at one time against a receptor. The methods described above can also be highly sensitive, which can enable the accurate detection and characterization of microscale samples. Furthermore, representative samples can be collected only when the material is actually in the sampling loop, as opposed to random sampling of eluant in a search for the desired products for analysis to elute out of the analytical device.

The processes described herein can provide a very general system for optimizing receptor-ligand interactions and can be applied to a large variety of types of protein classes and small molecule interactions including, but not limited to soluble proteins, membrane-associated proteins, enzymes, nuclear hormone receptors, and G-protein coupled receptors (GPCRs). Additionally, the process requires no biochemical assay for its output and utilizes only very small quantities of a purified protein or other biomolecule receptor, typically less than 5 μg per experiment. Moreover, the methods described herein do not require knowledge of the receptor's structure for its implementation. Further, the desired output of the methods can be achieved with mixtures of ligands (e.g., combinatorial libraries) and do not require purification of the ligands.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph depicting theoretical plots of ligand recovery of receptor-ligand complex ES versus initial ligand concentration [S]₀ for varying K_{d}.

FIG. 2 is a graph depicting theoretical plots of ligand recovery of receptor-ligand complex ES versus initial ligand concentration [S]₀ for varying initial active receptor concentration [E]₀.

FIG. 3 is a mathematical fit of ligand recovery data for receptor-ligand complex [ES] versus initial ligand concentration [S]₀ for theoretical data incorporating 10% random error.

FIG. 4 depicts theoretical plots of receptor-ligand complexes ESᵢ versus initial competitor ligand concentration [S₁]₀ for competition between a single compound S₂ and added competitor S₁.

FIG. 5A depicts a simulated ACE₅₀ experiment for a mixture of three ligands of different K_{d} under receptor-excess conditions: Solid lines show recovery for the given ligand at 1 μM total concentration; dashed boundary lines indicate recovery at 0.33 or 3.0 μM.

FIG. 5B depicts a simulated ACE₅₀ similar to that depicted in FIG. 5A, except receptor concentration is limiting (2 μM) with respect to total ligand concentration (15 μM).

FIG. 6 depicts a solution to formula (I).

FIG. 7 depicts theoretical plots of receptor-ligand complexes ESᵢ versus initial competitor ligand concentration [S₁]₀ for a three-component mixture.

FIG. 8 depicts normalized theoretical plots of receptor-ligand complexes ESᵢ versus initial competitor ligand concentration [S₁]₀ for a three-component mixture.

FIG. 9 depicts a simulated signal response (from an AS-MS experiment) of an allosteric-competitive titrant and ligand and the ratio thereof with increasing titrant concentration.

FIG. 10 depicts AS-MS-measured Akt-1-NGD-28835 and Akt-1-staurosporine complex concentrations and the ratio thereof with increasing titrant concentration. The asymptotically-bounded ratio plot indicates allosteric binding.

FIG. 11A depicts AS-MS-measured titrant and ligand responses from ACE₅₀ experiment between staurosporine and a mixture of ligands discovered by screening of mass-encoded combinatorial libraries.

FIG. 11B depicts titrant-ligand recovery ratios from the data in (C) indicating allosteric competition.

FIG. 11C-D depict respectively AS-MS-measured protein-ligand complex concentrations and ratio plots from ACE₅₀ experiment between M-1 and the mixture of ligands in (A) and (B). In contrast to the results with staurosporine, orthosteric binding competition is observed between the pool members and M-1.

FIGS. 12A-D are graphs depicting the inhibition of Akt-1 biochemical activity by staurosporine and M-1 under increasing ATP concentrations. 12A and 12B depict staurosporine results, showing competitive binding between staurosporine and ATP. 12C and 12D depict results for M-1, showing mixed, non-competitive binding between M-1 and ATP. Note: staurosporine is plotted vs. a nanomolar concentration axis, M-1 vs. a micromolar axis.

FIGS. 13A-B are graphs depicting affinity ranking and determination of the mode of binding competition vs. atropine for a pool of ligands to the muscarinic acetylcholine receptor M₂. 13A depicts a logarithmic plot of the ACE₅₀ experiment results indicating that NGD-3350 has higher affinity than its congeners NGD-3348 and NGD-3346 and other pool components. 13B depicts a linear titrant-ligand response ratio plot indicating orthosteric competition between atropine and the pool components.

FIG. 14 is a mathematical model of the effects on [ES] of a 1:1 dilution of an equilibrated solution of E+S at time zero with a high concentration of inhibitor I with association rate kᵢ₁ varying from zero to 0.1 μM⁻¹ sec⁻¹ (0→10⁵ M⁻¹sec⁻¹). Theoretical decay curve [E·S]₀e^{-ks2·t} shown as (E).

FIG. 15 is a log plot mathematical model of the effects on [ES] of a 1:1 dilution of an equilibrated solution of E+S at time zero with a high concentration of inhibitor I with association rate kᵢ₁ varying from zero to 0.1 μM−1 sec−1 (0→10⁵ M⁻¹sec⁻¹). Theoretical decay curve −[ES]₀kₛ₂·t (slope=kₛ₂) shown as (E).

FIG. 16 is a mathematical model of the effects on [ES] of a 1:1 dilution of an equilibrated solution of E+S at time zero with a high concentration of inhibitor I. kₛ₂ varies from 1 to 0.001 sec⁻¹; theoretical decay curve −[ES]₀kₛ₂·t (slope=kₛ₂) shown as (E).

FIG. 17 is a mathematical model of the effects on [ES] of a 1:1 dilution of an equilibrated solution of E+S at time zero with a high concentration of inhibitor, inh (I). Modeled kₛ₂=0.01 sec⁻¹ (A); theoretical decay curves shown for kₛ₂=0.01±0.001 sec⁻¹ (E).

FIG. 18 depicts a simulated affinity selection-mass spectrometry response for and ratios of receptor-ligand complexes [ESᵢ] and [ESᵥ] from an equilibrated solution of E+Sᵢ+Sᵥ as a function of receptor concentration [E]₀ from receptor-excess to receptor-limiting conditions.

FIG. 19 is a flow diagram depicting the path of a ligand of a receptor-ligand complex.

FIGS. 20A-20B depict a multiport valve at two different positions, 20A and 20B.

FIG. 21 depicts UV data for four consecutive sample injections at a wavelength of 230 nm.

FIGS. 22A-22F depict the effects of a change in the peak cut delay time of the multiport valve.

FIG. 23 is a mathematical fit of ligand recovery data for receptor-ligand complex [ES] versus initial ligand concentration [S]₀ for Human Serum Albumin+Warfarin.

FIG. 24 depicts normalized plots of ligand recovery for receptor-ligand complexes ESᵢ versus initial competitor ligand (Atropine) concentration [S₁]₀ for a five-component mixture vs. Porcine Muscarinic Receptor and their derived ACE₅₀ values.

FIG. 25 depicts normalized plots of ligand recovery for receptor-ligand complexes ESᵢ versus initial competitor ligand (Atropine) concentration [S₁]₀ for two selected ligands from a five-component mixture and their K_{d}'s.

FIG. 26 depicts the graphical solution yielding the k₂ and half-life of Zap-70 complexes Zap-70•NGD-746 and Zap-70•NGD-6380.

FIG. 27 depicts the graphical solution yielding the k₂ and half-life of Zap-70 complexes with the mixture of ligands.

FIG. 28 depicts normalized affinity selection-mass spectrometry responses for receptor-ligand complexes from an optimization library versus total competitor ligand Staurosporine concentration and derived ACE₅₀ and K_{d} values, Staurosporine K_{d}=100 nM.

### DETAILED DESCRIPTION

U.S. Pat. No. 6,207,861 relates to methods for making mass-coded combinatorial libraries and methods of identifying (i.e., screening) compounds in those mass-coded combinatorial libraries that associate with one or more biomolecules. The mass-coded libraries are designed and synthesized such that at least about 90% (i.e., any integer percent between 90-100, inclusive) of the individual compounds have a molecular mass that is distinct from the molecular masses of the other individual compounds in the mass-coded library.

The screening methods in U.S. Pat. No. 6,207,861 describe a system referred to as the Automated Ligand Identification System (ALIS). The ALIS system generally functions as follows: (1) a solution of the biomolecule of interest (e.g. a protein) is incubated in the presence of a ligand (e.g. a small organic molecule or library thereof) for a prescribed length of time to allow the biomolecule-ligand complex forming reaction to reach equilibrium; (2) the solution of biomolecule, unbound ligand and biomolecule-ligand complex is passed through a size-exclusion chromatography stage to separate the biomolecule plus biomolecule-ligand complex from unbound ligand on the basis of molecular size, with the biomolecule plus biomolecule-ligand complex co-eluting at the front of the eluant stream; (3) the portion of the eluant stream containing the biomolecule plus biomolecule-ligand complex but not containing any unbound ligand is subjected to a reverse-phase chromatography stage for desalting and elution into a mass spectrometer where the organic molecule may be identified on the basis of its molecular weight or mass spectrometry-mass spectrometry fragmentation pattern and quantified by measurement of its calibrated signal response.

The methods described herein apply the technology described in U.S. Pat. No. 6,207,861, and are also based, in part, on the discovery that quantitative analysis of receptor-ligand pairs can be accomplished by analysis of the signal response from an analytical device or detector (e.g., mass spectrometer, HPLC, UV, IR, NMR, etc.), which corresponds to the ligand recovery for ligand-binding experiments performed over a range of ligand concentrations, where the ligand recovery is based on the separation of unbound and bound ligands (e.g., by size-exclusion chromatography, gel filtration, ultracentrifugation). These experiments yield data that can be mathematically fit to models describing the kinetics and equilibrium thermodynamics of receptor-ligand binding. The models can provide estimates of equilibrium dissociation constants (K_{d}), initial receptor concentrations [R]₀, and absolute rates of dissociation (off-rates, k_{off}) for mixtures of receptors and ligands.

The term “equilibrium dissociation constant” (K_{d}) refers to the ratio of the reverse and forward rate constants for a reaction of the type E+S=ES. At equilibrium, the equilibrium dissociation constant (K_{d}) equals the product of the concentrations of reactants divided by the concentration of product (K_{d}=[E][S])/[ES]). The smaller the value of K_{d}, the stronger is the binding interaction between the associating reactants. The affinity (association) constant is the reciprocal of the equilibrium constant.

The reversible binding of a ligand to a receptor is denoted as follows:

$$E + S {{⇄}^{{k}_{on}}}_{{k}_{off}} E S$$

K_{d} is denoted as follows:

$${K}_{d} = \frac{{k}_{off}}{{k}_{on}} = \frac{[ E ] [ S ]}{[ E S ]}$$

In some instances, kₒₙ is depicted as k₁ and k_{off} is depicted as k₂.

Total initial receptor concentration, [E]₀, can be defined as the concentration of free receptor, [E], plus the concentration of bound receptor [ES], ([E]₀=[E]+[ES]). The total ligand concentration, [S]₀, can be defined as the concentration of free ligand plus the concentration of bound ligand ([S]₀=[S]+[ES]).

Accordingly, K_{d} can be expressed in terms of the equilibrium complex concentration [ES] and initial receptor and substrate concentrations [E]₀ and [S]₀ as follows:

$${K}_{d} = \frac{( {[ E ]}_{0} - [ ES ] ) ( {[ S ]}_{0} - [ ES ] )}{[ ES ]} .$$

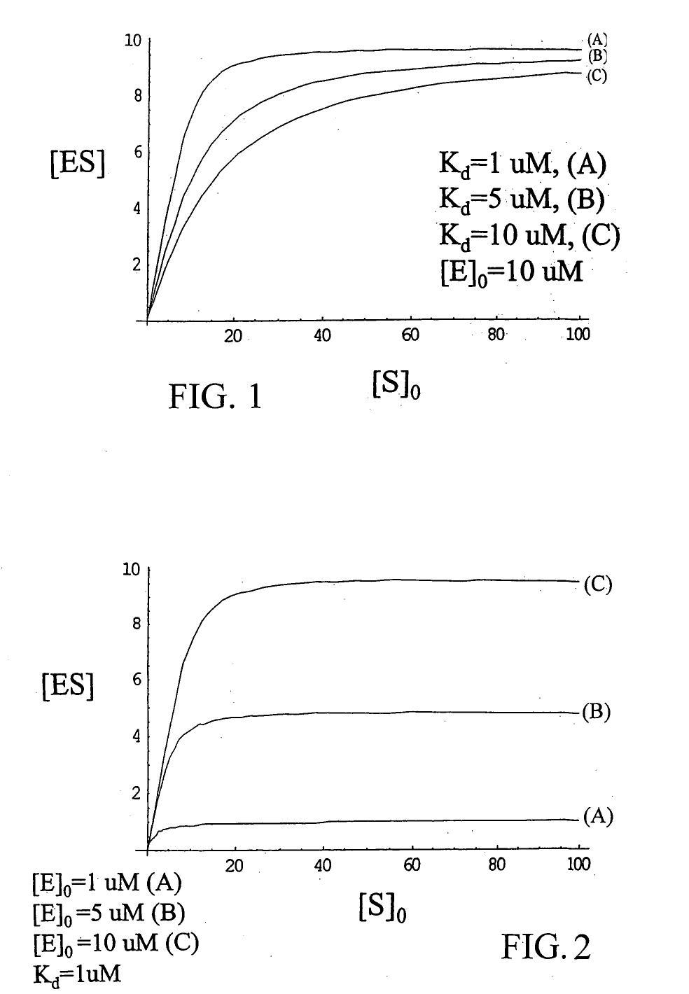

To determine K_{d}, the equilibrium complex concentration [ES] initial receptor and substrate concentrations [E]₀ and [S]₀ must either be known or determined.

[ES] can be measured experimentally (e.g., using size-exclusion chromatography to separate the bound ligand from the unbound ligand, followed by liquid chromatography wherein the receptor-ligand complex is disrupted, followed further by mass spectroscopy wherein the mass spectrometer has been calibrated for the ligand).

[S]₀ can be known or estimated.

[E]₀ can vary depending on conditions and is determined experimentally. To measure [E]₀, a ligand can be titrated against the receptor. The recovery of complex [ES] reaches a maximum at the value of [E]₀, and the corresponding titration curve can be fit by the following equation:
[ES]²−[ES](K_{d}+[E]₀+[S]₀)+[E]₀[S]₀=0
the outcome of which describes the equilibrium concentration of receptor-ligand complex ES as a function of the parameters K_{d}, [S]₀, and [E]₀. For a given K_{d} and [E]₀, a plot of [ES] vs. [S]₀ follows a rectangular hyperbolic curve as shown in FIG. 1. The precise shape of this curve depends on the K_{d} for the ligand under study. As shown in FIG. 2, the asymptotic limit to [ES] is dependent on [E]₀. This behavior is described as saturation binding.

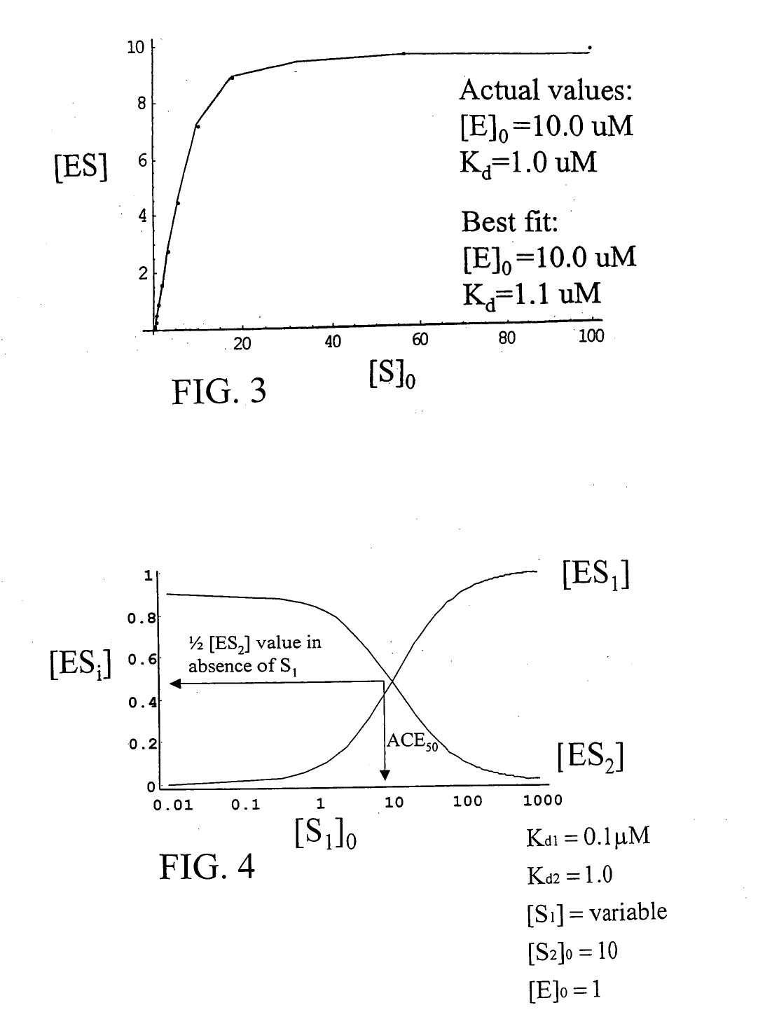

Given a set of [ES] values measured by calibrated experiments at different values of [S]₀, the equation can be fit to the [ES], [S]₀ data pairs to yield best-fit values for the parameters K_{d} and [E]₀ by numerical non-linear regression techniques (See FIG. 3).

The same expressions used to describe the interaction of a single ligand to a single receptor can also be used to describe the interacting of a mixture of compounds (e.g., ligands) with a single receptor-binding site, demonstrated as follows:
E+S₁+S₂+ . . . Sᵢ→ES₁+ES₂+ . . . ESᵢ.

The K_{d} for each ligand Sᵢ can be expressed as follows:

$${K}_{d i} = \frac{[ E ] [ {S}_{i} ]}{[ E {S}_{i} ]}$$

which expands by analogy to the expression of K_{d} expressed above in the case of a single ligand to

$${K}_{d i} = \frac{( [ {E}_{0} - {\sum }_{i} [ E {S}_{i} ] ) ( {[ {S}_{i} ]}_{0} - [ E {S}_{i} ] )}{[ E {S}_{i} ]} \qquad formula ( I )$$

Simultaneous solution of formula (I) for a theoretical two-component mixture over a range of concentrations of a ligand S₁ and at a fixed concentration of the second ligand S₂ yields a graphical representation of receptor-ligand complex concentrations [ES₁] and [ES₂] shown in FIG. 4 for the conditions therein annotated. As the initial concentration [S₁]₀ of the known ligand S₁ is increased, the corresponding receptor-ligand complex concentration [ES₁] increases. However, when S₁ and S₂ bind competitively (e.g., orthosteric binding) to the receptor, the receptor-ligand complex concentration [ES₂] decreases because S₁ at higher concentration outcompetes S₂ for the receptor E's binding site.

In instances where a plurality of ligands are present in a mixture, a first ligand having a relatively weak binding affinity for a receptor relative to a second ligand will be competed off at lower concentrations of a titrant (e.g., a known competitive ligand) than a second ligand. For example, when titrating a known inhibitor of an enzyme in the presence of a plurality of unknown ligands, the relative binding affinities can be determined by assessing the order in which the ligands are displaced from the enzyme by a competitive ligand.

The term “ACE₅₀” refers to the concentration of a competitor compound for a ligand of interest required to reduce a receptor-ligand complex's concentration to ½ (50%) of its value compared to in the absence of the competitor. The ACE₅₀ depends on parameters such as the equilibrium binding constant K_{d} for the ligand of interest and of the competitor, as well as the concentrations of the receptor and the ligand. The ACE₅₀ value, which describes the concentration of the known compound as it competes out the ligand of interest, is the converse of the ordinary definition of a biochemical or biophysical IC₅₀, which describes the concentration of the ligand of interest as it competes out a known compound, e.g. a radioligand. In contrast to a conventional IC₅₀ value, a higher ACE₅₀ value indicates a higher-affinity ligand: greater competitor concentration is required to displace the compound of interest from the binding site.

Under experimental conditions where a receptor, E, is present in excess, relative to a pool of ligands of interest, Sᵢ, and the receptor-ligand pool mixture is titrated with another competitor ligand, competition between the components of the pool is considered inconsequential with the only apparent competition observed for a given ligand, Sᵢ, being the competition between the ligand, Sᵢ, and the added competitor.

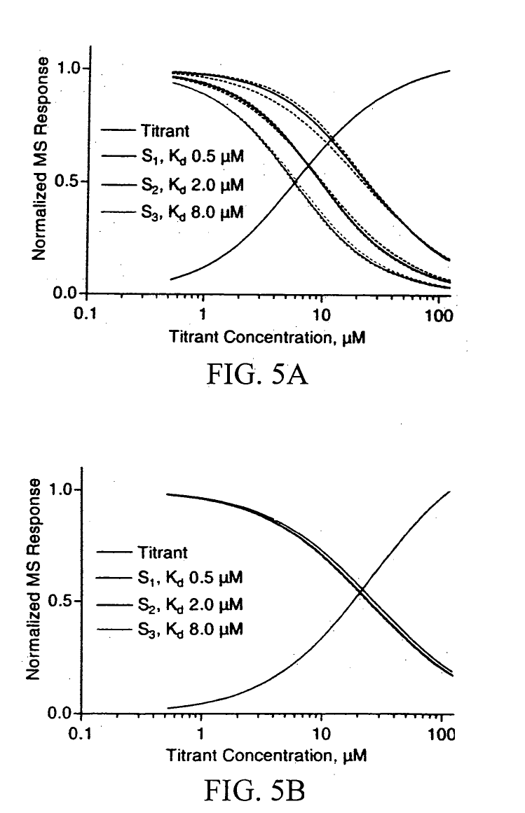

A simulated binding displacement experiment is shown in FIG. 5a for a mixture of three ligands of varying K_{d}, demonstrating how the ACE₅₀ method may be used to simultaneously affinity-rank multiple compounds. In this simulation the total concentration of all pool components ([S₁]₀=[S₂]₀=[S₃]₀=1.0 μM) is on the order of the total receptor concentration (5.0 μM). Under these conditions inter-library competition is limited and the individual library components compete only with the titrant. The ACE₅₀ values are insensitive to ligand concentration: nine-fold variation of the concentration of any one ligand yields the same rank order of affinity. This feature of the method can be beneficial when directly evaluating synthetic mixtures where ligand concentration may vary due to differences in building block reactivity. The consequence of higher pool concentration than receptor concentration is demonstrated in FIG. 5b. In the extreme case shown in this simulation, three ligands of different K_{d} can yield nearly equivalent ACE₅₀ values when the total ligand concentration of each of the three library components is 5 μM and the receptor concentration is 2 μM.

Extracting K_{d} information from ACE₅₀ data derived from a mixture of ligands reduces to a solution for a binary mixture of a ligand, Sᵢ, and the added competitor.

Using the principles described above, the K_{d} expression (i.e., K_{di}) given a total receptor concentration, [E₀] and the total ligand concentration [Sᵢ]₀ for a ligand Sᵢ is expressed in formula (I) below.

$${K}_{d i} = \frac{( {[ E ]}_{0} - {\sum }_{i} [ E {S}_{i} ] ) ( {[ {S}_{i} ]}_{0} - [ E {S}_{i} ] )}{[ E {S}_{i} ]} \qquad formula ( I )$$

In the absence of competitor ligand Sᵢ, the K_{d} expression (i.e., K_{di}) for ligand S₂ simplifies to the equation shown in formula (II) below.

$${K}_{d 2} = \frac{( {[ E ]}_{0} - [ E {S}_{2} ] ) ( {[ {S}_{2} ]}_{0} - [ E {S}_{2} ] )}{[ E {S}_{2} ]} \qquad formula ( II )$$

For simplification and readability, in the equations below the following nomenclature is used.
kd1=K_{d1}
kd2=K_{d2}
s10=[S₁]₀
s20=[S₂]₀
e0=[E]₀
es1=[ES₁]
es2=[ES₂]

The solution of formula (II) for [ES2] yields two roots, the real one being that shown below in formula (III):

$$es2 = \frac{1}{2} ( e0 + kd2 + s20 - \sqrt{- 4 e0s20 + {( e0 + kd2 + s20 )}^{2}} ) . \qquad formula ( III )$$

This expression represents the value of [ES₂] in the absence of competitor S₁. One half of this value is, by definition, the amount of [ES₂] present where the total concentration of competitor S₁, [S₁]₀, is equal to ACE₅₀.

The solution of formula (III) for [ES₂] yields two roots, the real one being that shown below in formula (IV):

$$es2 = \frac{1}{2} ( e0 - es1 + kd2 + s20 - \\ \sqrt{{( e0 - es1 + kd2 + s20 )}^{2} + 4 ( - e0s20 + es1s20 )} ) \qquad formula ( IV )$$

This expression yields the value of [ES₂] when in competition with competitor S₁. Setting the right hand side (rhs) of formula (IV) equal to ½ of the rhs and solving for [ES₁] yields the following formula (V):

$$es1 = \frac{1}{4 e0 + 8 kd2 - 8 s20} \\ ( 3 {e0}^{2} + 5 e0 kd2 + 2 {kd2}^{2} - 7 e0s20 + 4 kd 2 s20 + 2 {s20}^{2} + \\ e0 \sqrt{- 4 e0s20 + {( e0 + kd2 + s20 )}^{2}} - \\ 2 kd2 \sqrt{- 4 e0s20 + {( e0 + kd2 + s20 )}^{2}} - \\ 2 s20 \sqrt{- 4 e0s20 + {( e0 + kd2 + s20 )}^{2}} ) . \qquad formula ( V )$$

Formula (V) represents the value of [ES₁] where [ES₂] is ½ its value in the absence of competitor S₁; in other words, this is the value of [ES₁] where [S₁]₀=ACE₅₀.

In analogy, the solution for [ES₁] yields two roots, the real one being that shown in formula (VI) below:

$$es1 = \frac{1}{2} ( e0 - es2 + kd1 + s10 - \sqrt{\begin{matrix}{( e0 - es2 + kd1 + s10 )}^{2} + \\ 4 ( - e0s10 + es2s10 )\end{matrix}} ) . \qquad formula ( VI )$$

Substitution of the value of [ES₂] at ACE₅₀, which is ½ of the rhs of formula (III) into the expression for the value of [ES₁] when in competition with competitor S₂, which is formula (VI) yields the following formula (VII):

$$es1 = \frac{1}{2} ( e0 - ( \frac{1}{4} ( e0 + kd2 + s20 - \sqrt{- 4 e0s20 + {( e0 + kd2 + s20 )}^{2}} ) ) \cdot kd1 + s10 - √ ( {( e0 - ( \frac{1}{4} ( e0 + kd2 + s20 - \sqrt{- 4 e0s20 + {( e0 + kd2 + s20 )}^{2}} ) ) + kd1 + s10 )}^{2} + 4 ( - e0s10 + ( \frac{1}{4} ( e0 + kd2 + s20 - \sqrt{- 4 e0s20 + {( e0 + kd2 + s20 )}^{2}} ) 0 * s10 ) ) ) . \qquad formula ( VII )$$

Both formulas (V) and (VII) above are expressions for [ES₁] at [S₁]₀=ACE₅₀. Setting the rhs of formula (V) equal to the rhs of formula (VII) and solving for [S₁]₀ yields the following expression, which is the value of [S₁]₀ where [ES₂] is ½ its value in the absence of a competitor; in other words, [S₁]₀ in the expression below is equal to ACE₅₀, and is expressed in terms of the K_{d}'s of the competitor K_{d1} and the ligand of interest K_{d2}, the total receptor concentration [E]₀ and the total concentration of the ligand being titrated [S₂]₀:

$$ACE50 = s10 = \frac{1}{4 kd2 ( e0 + 2 kd2 - 2 s20 )} ( {e0}^{2} ( kd1 + 3 kd2 ) + e0 ( kd2 ( 5 kd2 - 7 s20 + \sqrt{- 4 e0s20 + {( e0 + kd2 + s20 )}^{2}} ) + kd1 ( 3 kd2 - s20 + 3 \sqrt{- 4 e0s20 + {( e0 + kd2 + s20 )}^{2}} ) ) + 2 ( kd2 ( kd2 + s20 ) ( kd2 + s20 - \sqrt{- 4 e0s20 + {( e0 + kd2 + s20 )}^{2}} ) + kd1 ( kd2 - s20 ) ( kd2 + s20 + 3 \sqrt{- 4 e0s20 + {( e0 + kd2 + s20 )}^{2}} ) ) ) . \qquad formula ( VIII )$$

Formula (VIII) allows a user to predict the value of ACE₅₀ for a given set of experimental parameters K_{d1}, K_{d2}, E₀, and [S_{2]}₀. Additionally, this expression can be used to optimize the parameters for ACE₅₀ determination.

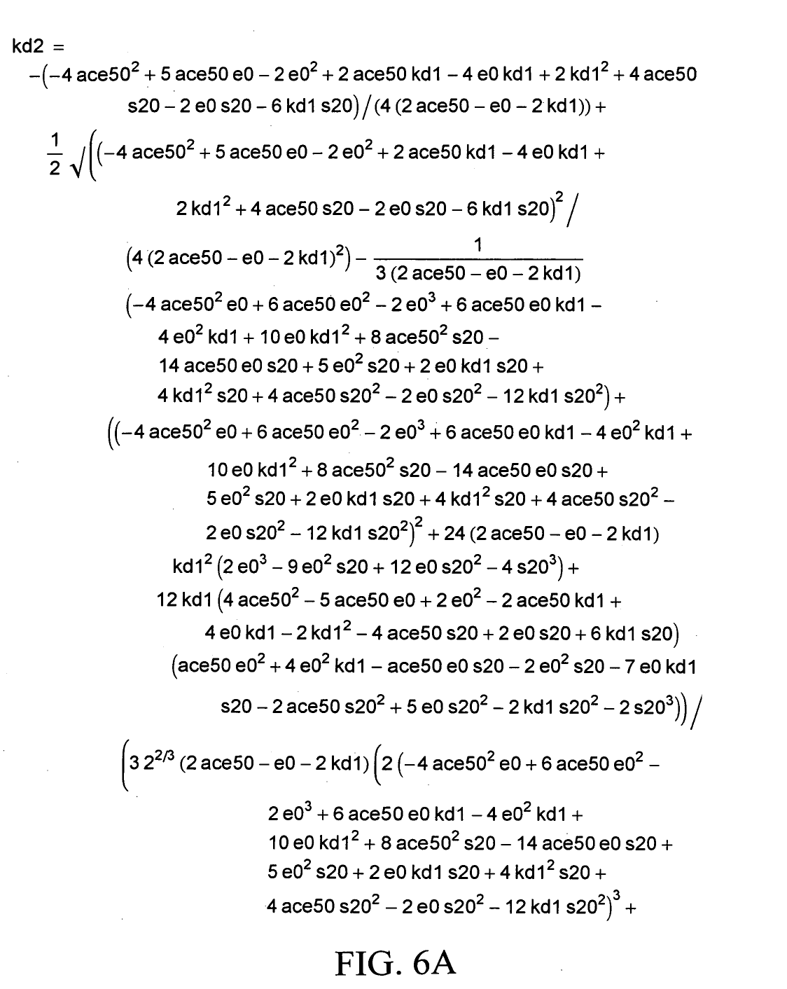

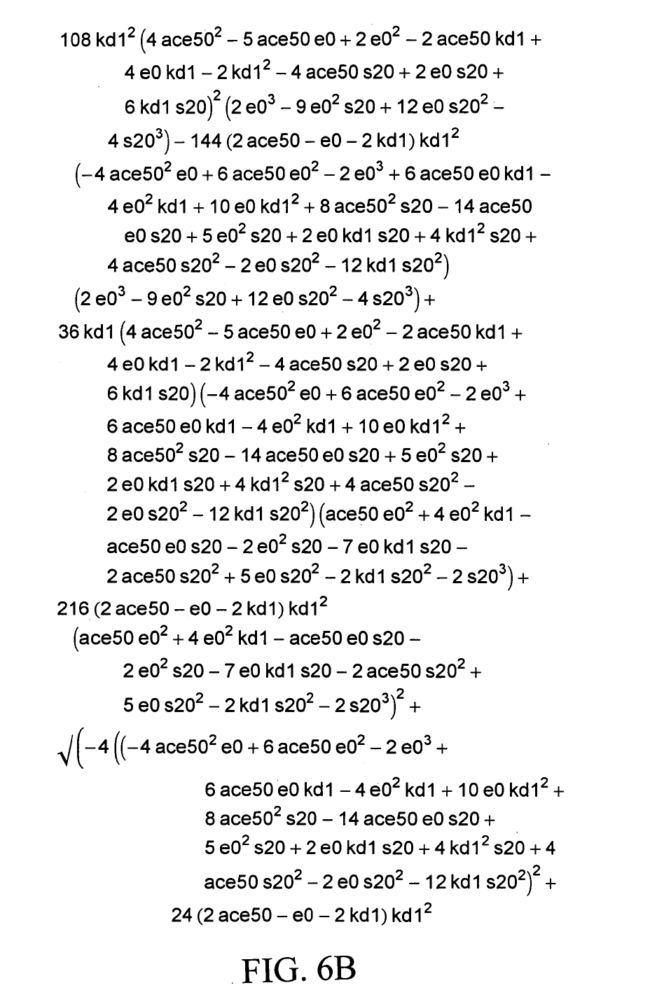

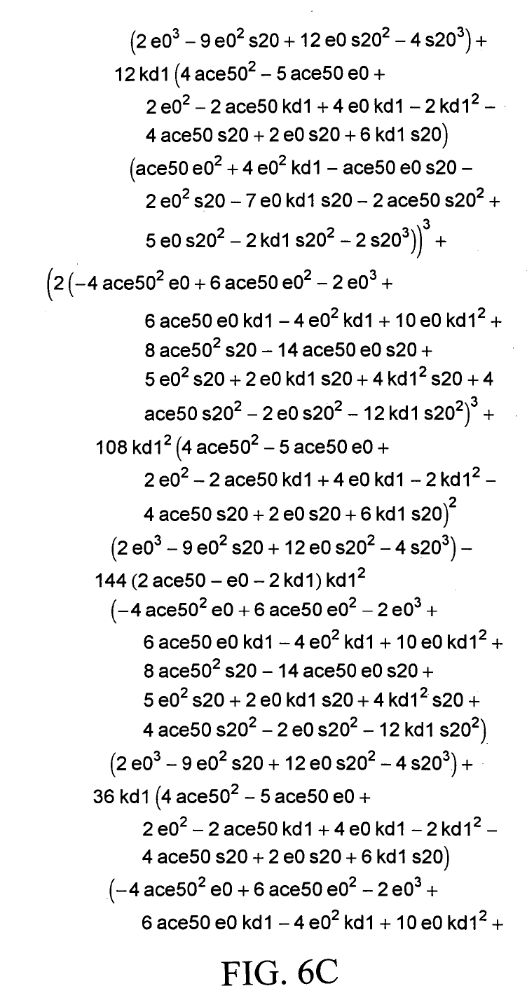

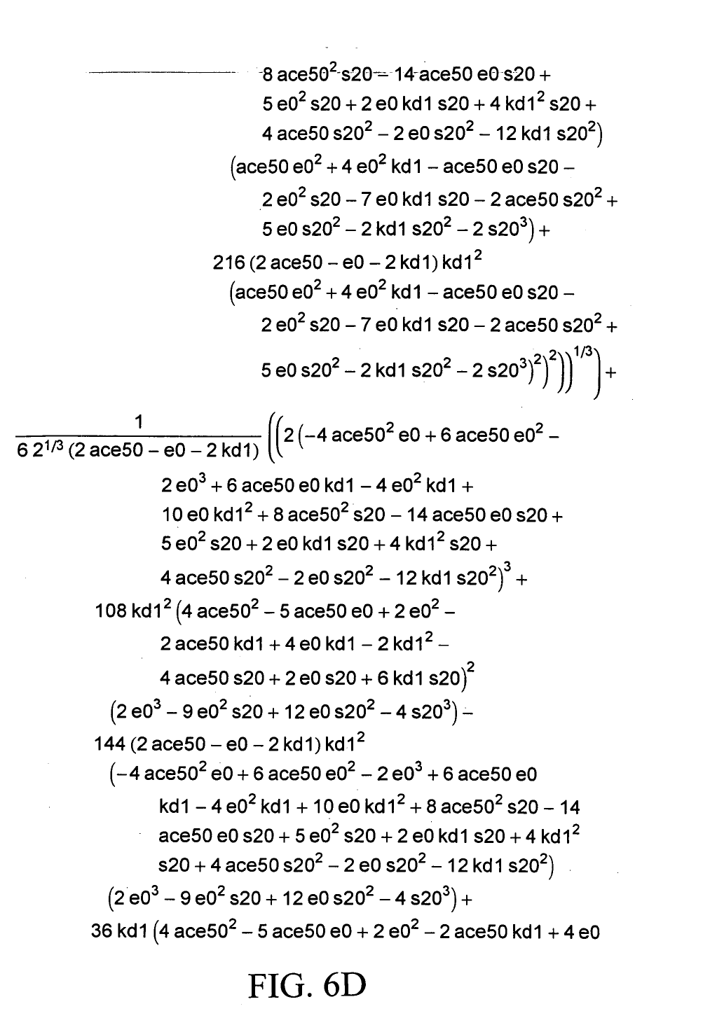

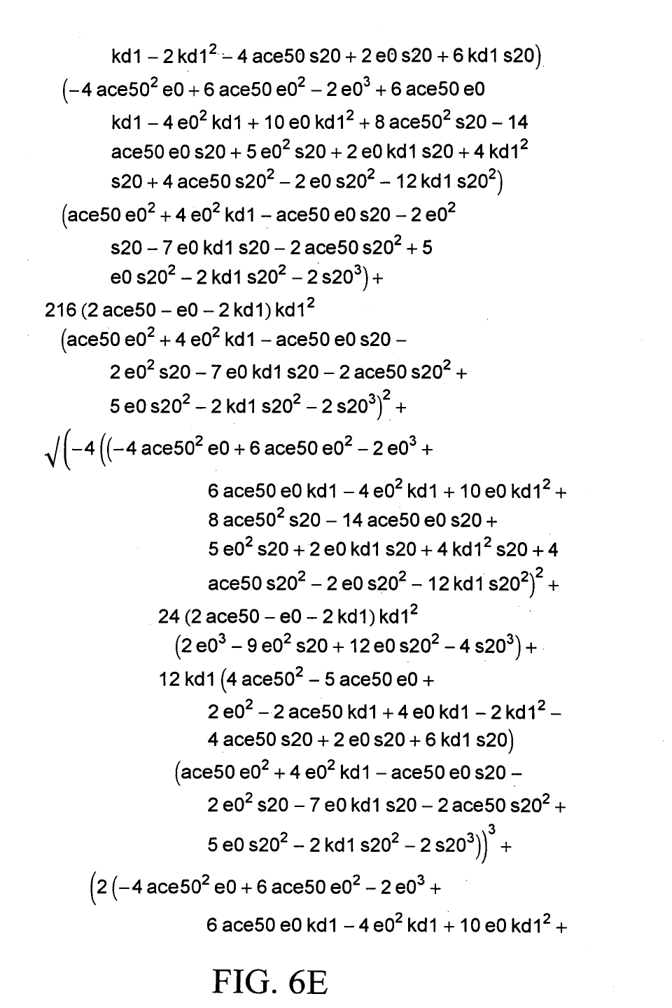

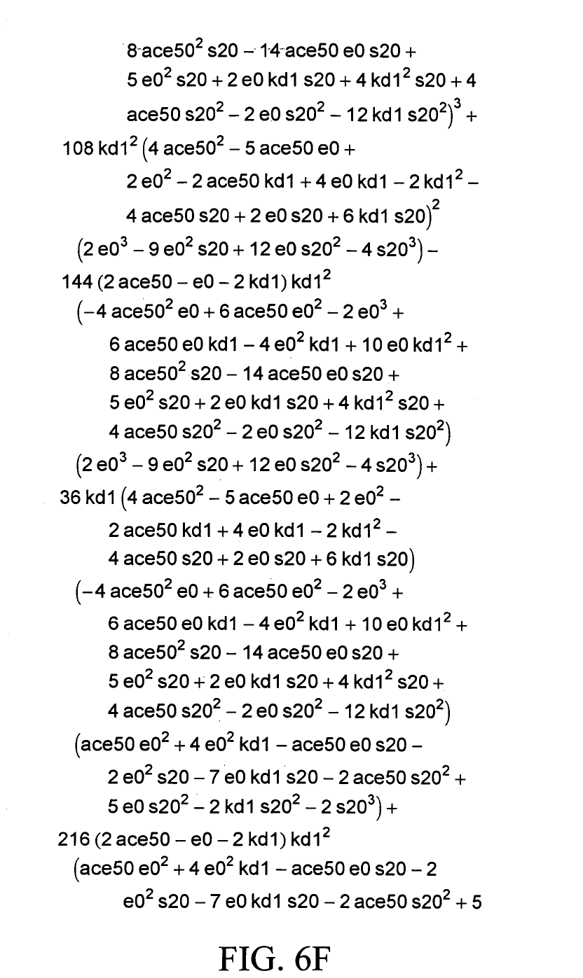

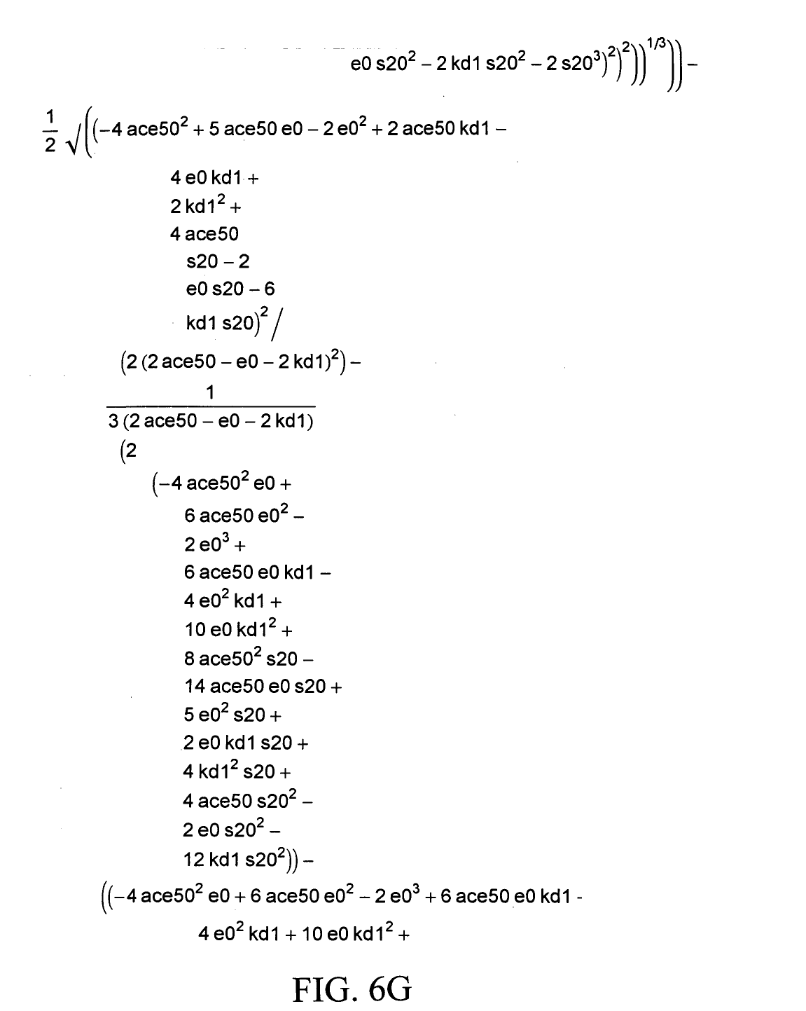

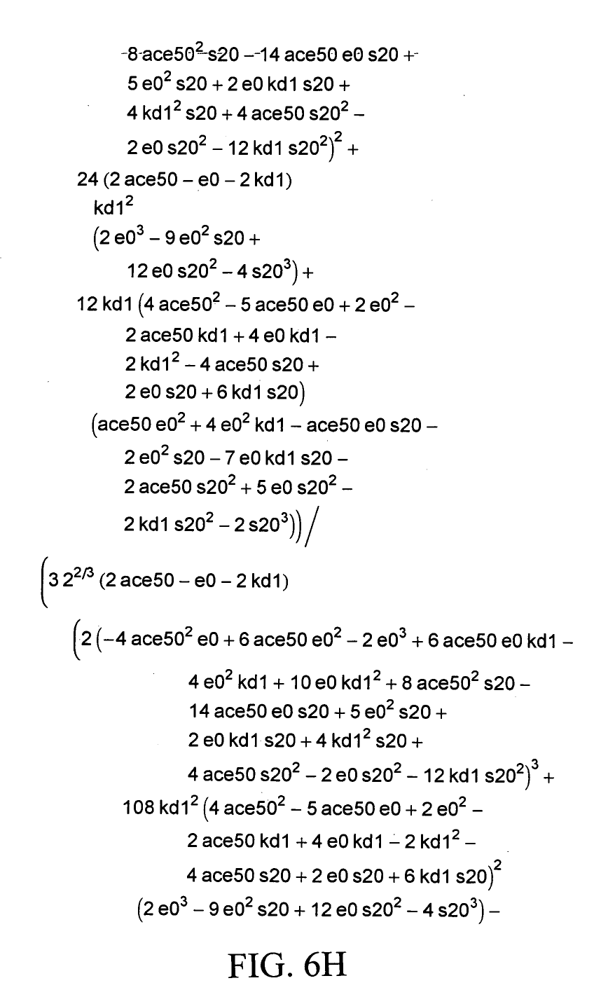

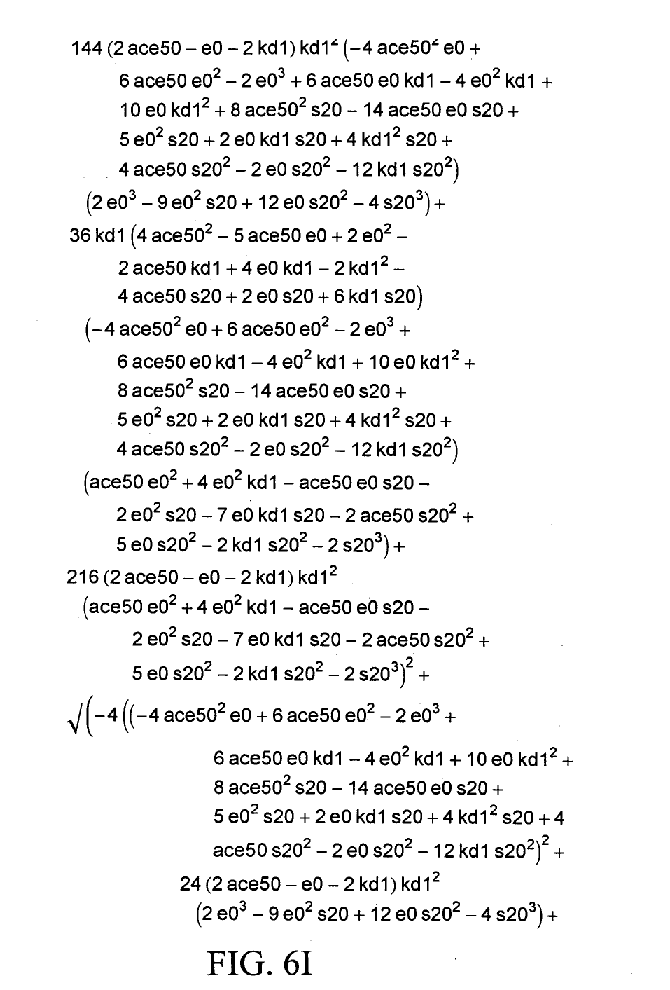

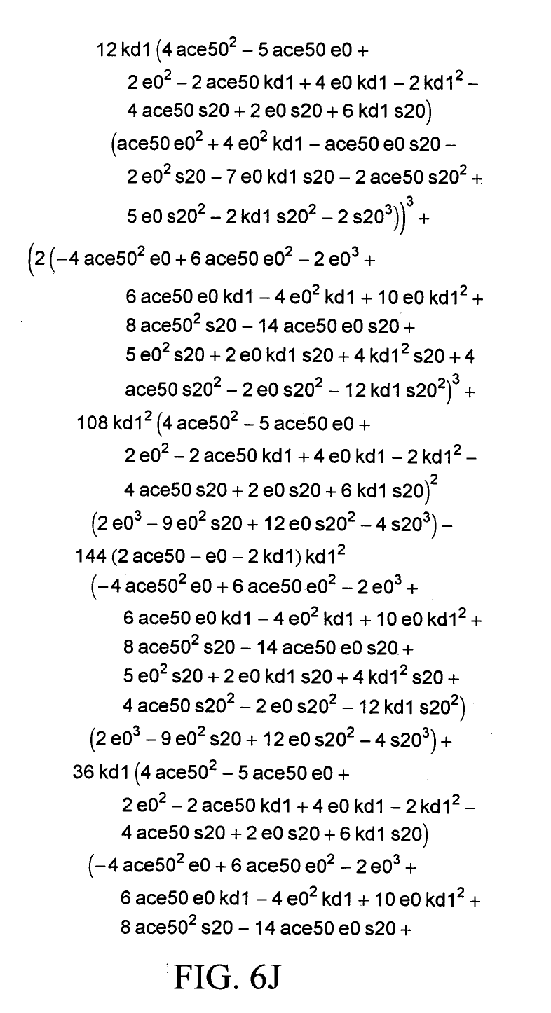

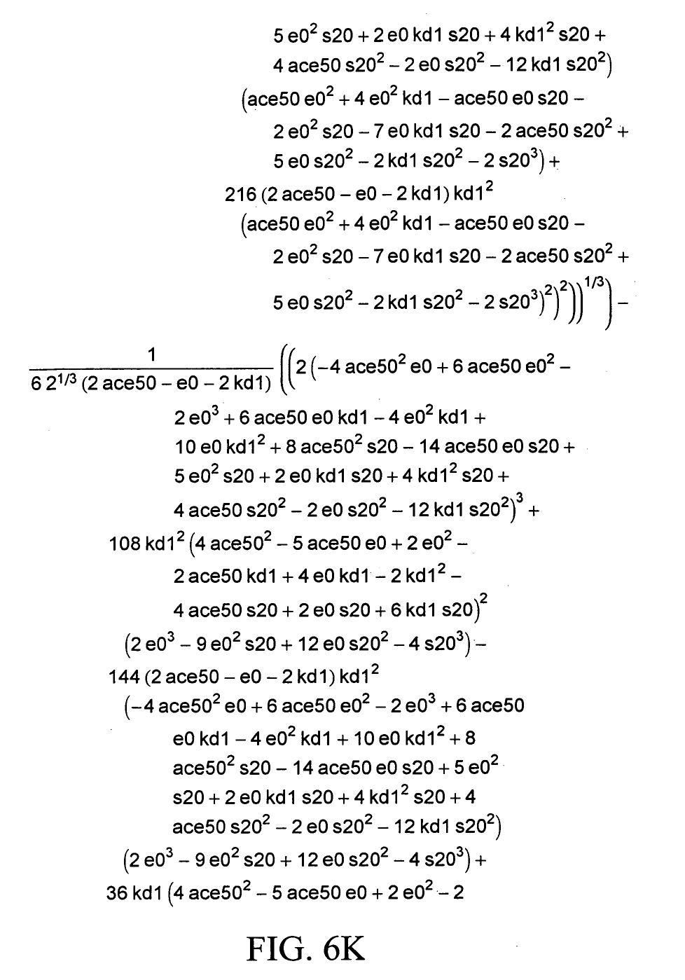

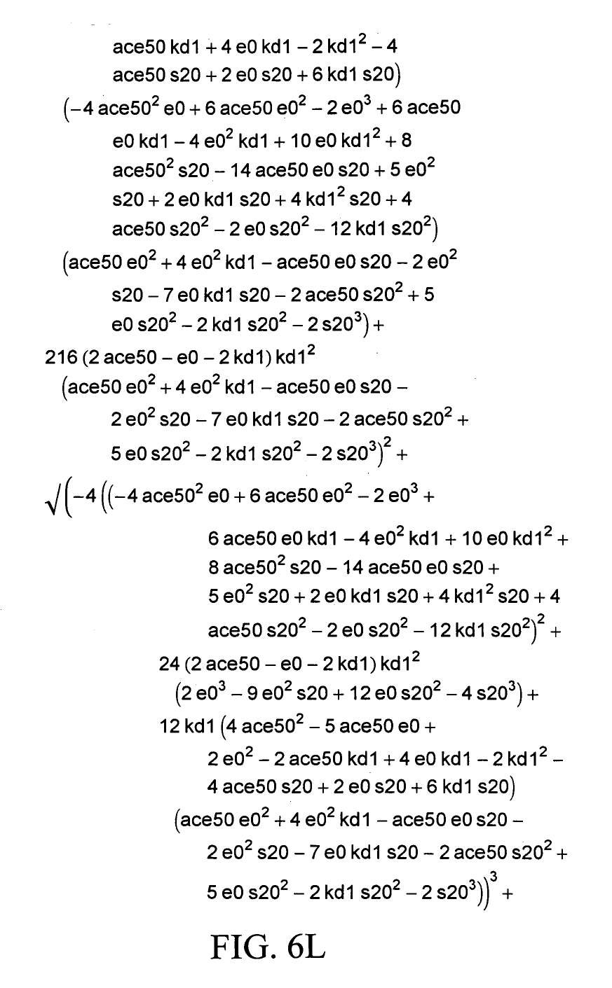

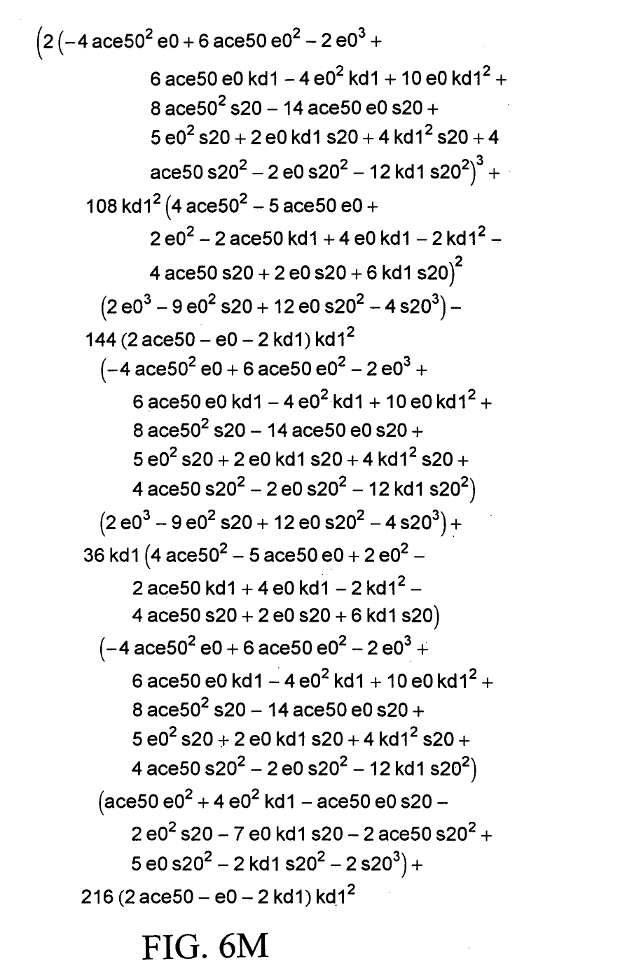

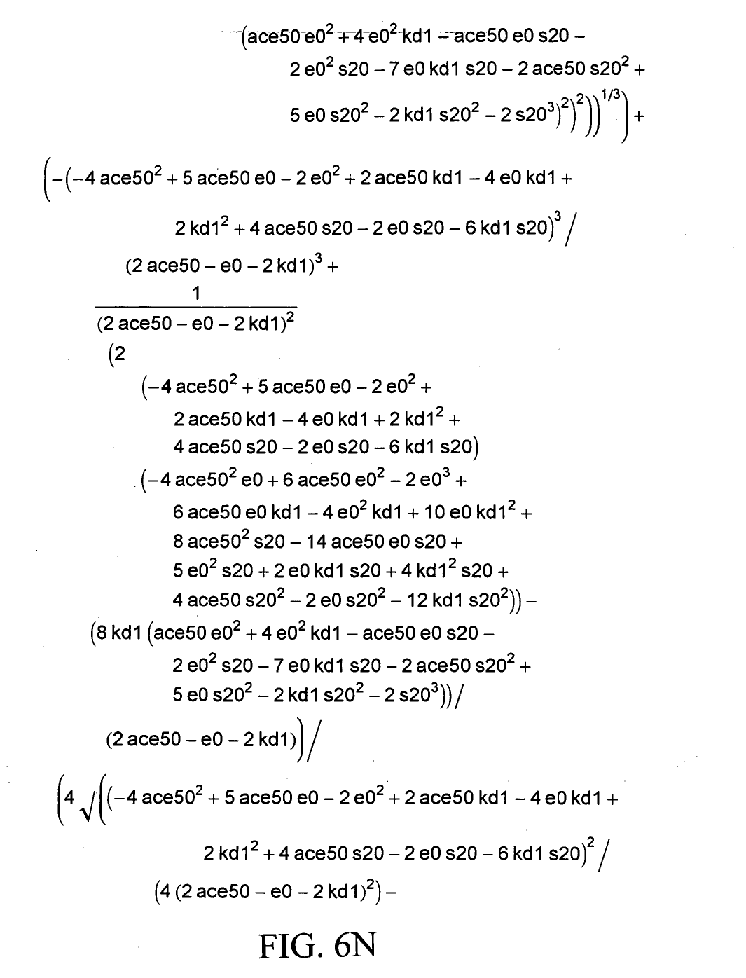

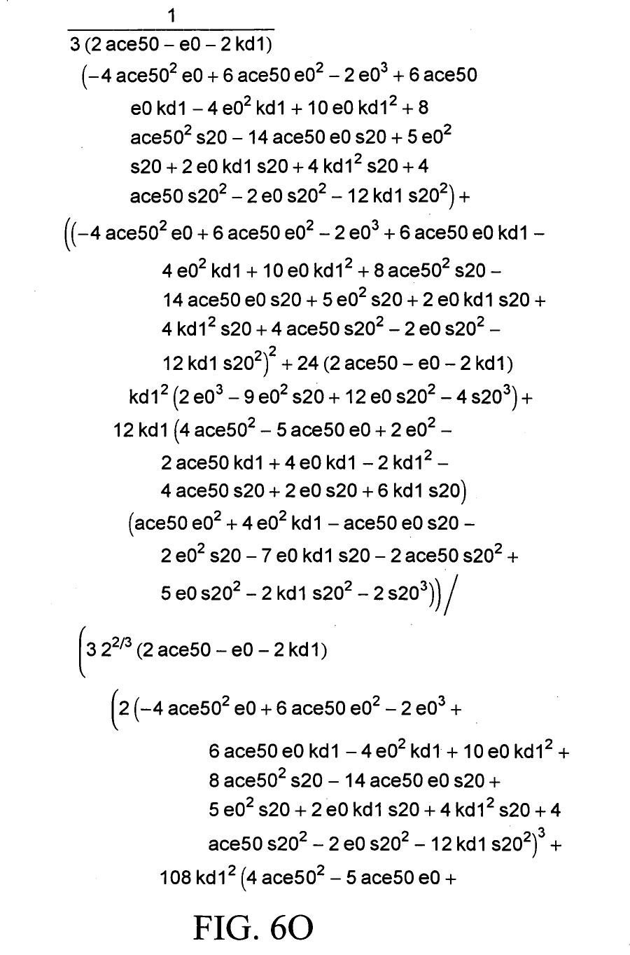

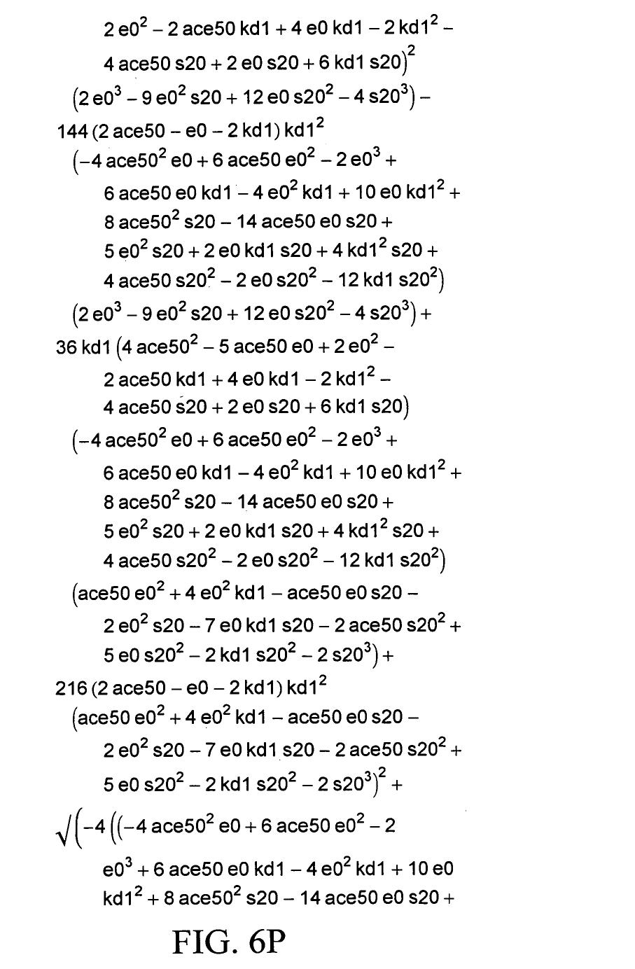

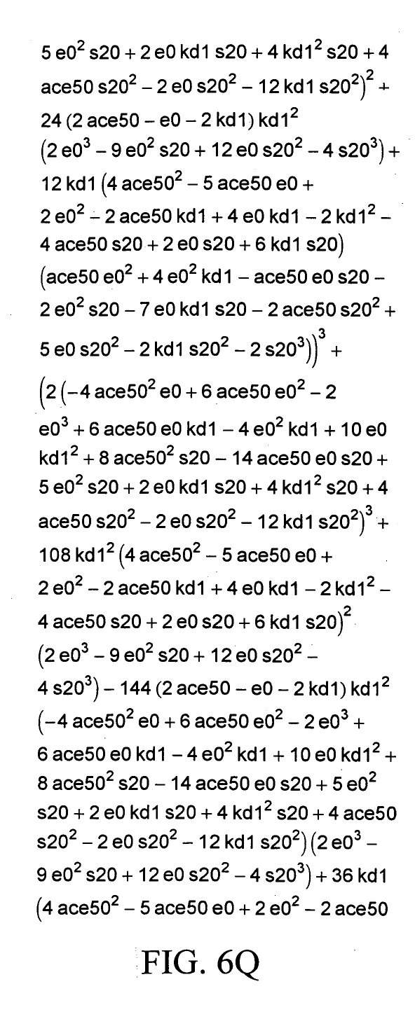

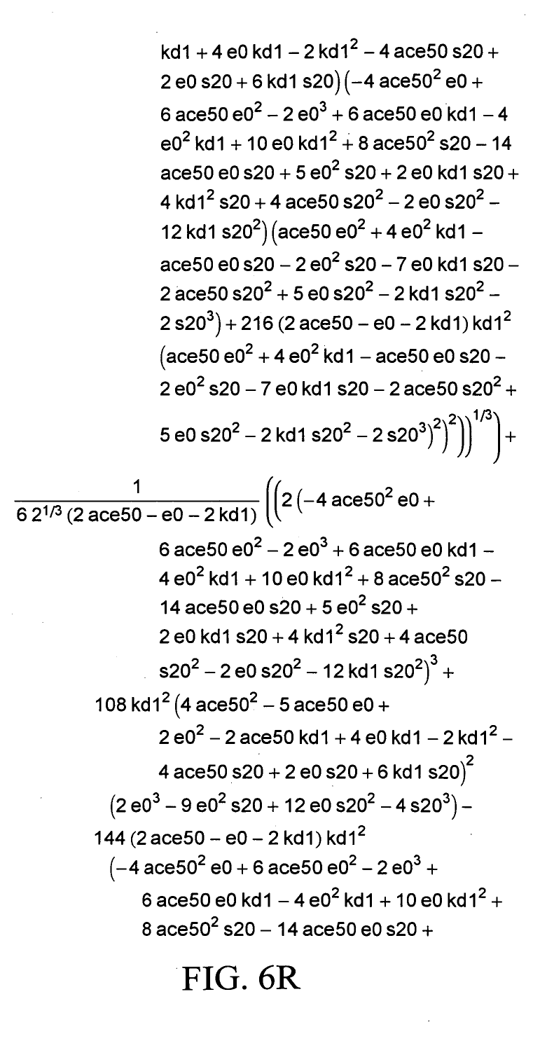

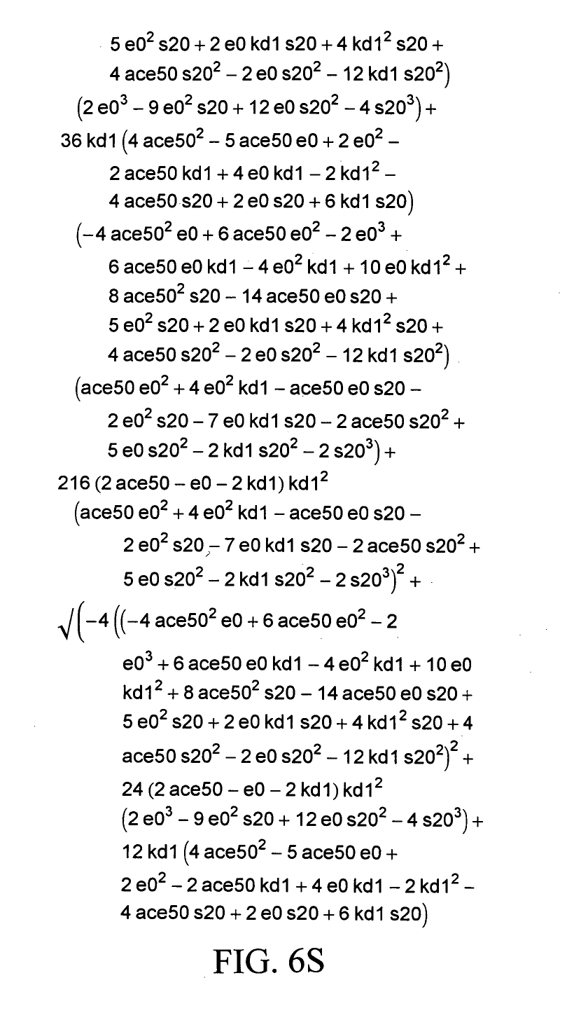

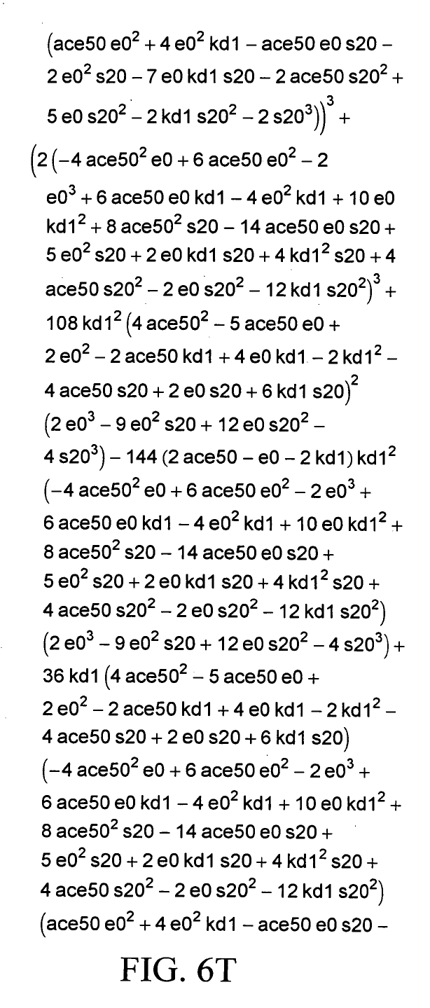

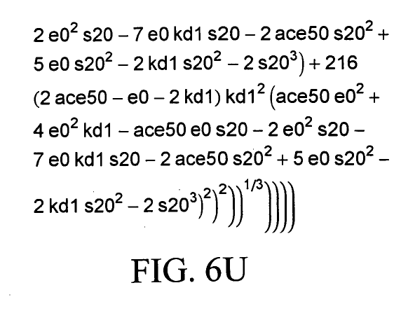

Solution of formula (VIII) for K_{d2} yields four roots, the real one of which is an expression for K_{d2} in terms of the ACE₅₀, the K_{d} of the competitor K_{d1}, the total receptor concentration E₀ and the total concentration of the ligand being titrated [S_{2]}₀. As shown in FIG. 6, the exact solution to formula (I) is unwieldy, and an alternate approach for deriving the value of K_{d2} from the experimental values of ACE₅₀, K_{d1}, E₀, and [S_{2]}₀ is the numerical solution of formula (VIII) for K_{d2} using ACE₅₀, K_{d1}, E₀, and [S₂]₀ as input parameters.

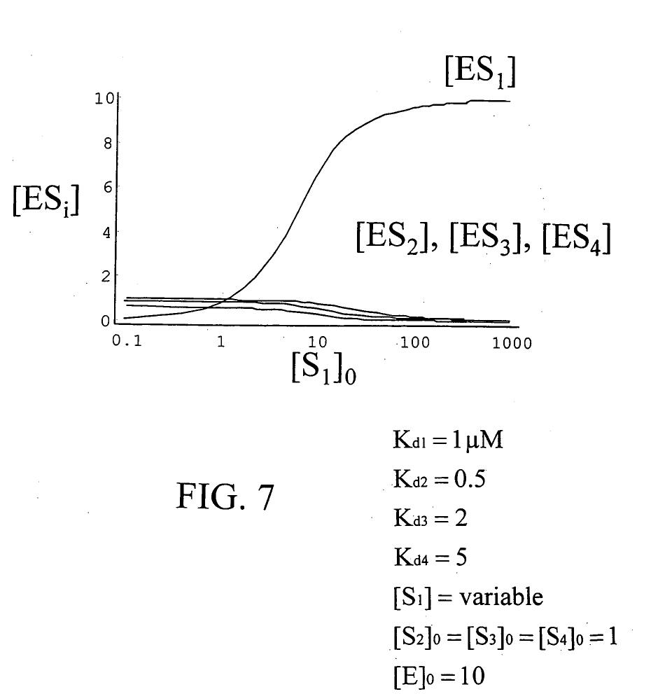

FIG. 7 shows the results of a theoretical titration of a ternary mixture of compounds S₁, S₂, and S₃ with a known ligand S₁. Note that the total concentration of the ligands (except the added competitor) is less than the total receptor concentration. In these circumstances, the compounds in the mixtures may be considered to be non-competitive with each other, and only compete with the ligand, which is added in excess.

In the presence of 2.0 μM orthosteric-competitive ligand S₂ with K_{d2}=0.5 μM, titration by S₁ yields a shallower binding curve for E·S₁, and the concentration of protein-ligand complex E·S₂ decreases as the receptor is saturated by S₁. Where the ratio of E·S₁ to E·S₂ increases linearly with total titrant concentration [S₁]₀ when the receptor is the limiting reagent (i.e., when the titrant and ligand must compete for the receptor site). This linear relationship indicates mutually exclusive competitive binding, most simply explained by orthosteric interaction with the receptor, and is described by the following equation:

$$\frac{E \cdot {S}_{1}}{E \cdot {S}_{2}} = \frac{{[ {S}_{1} ]}_{0} {K}_{d2}}{{[ {S}_{2} ]}_{0} {K}_{d1}}$$

Therefore, a plot of the ratio of ligand recoveries for E·S₁ to E·S₂ versus total titrant concentration [S₁]₀ will yield a straight line for orthosteric ligands where [S₁]₀>[E]₀. If the MS response calibration factors of the titrant and ligand are known, the slope of this line corresponds to the ratio of ligand and titrant affinities divided by the concentration of the ligand.

The term “orthosteric” as used herein to describe ligands binding to a receptor, refers to ligands that bind in the same receptor site and bind, generally, in a mutually exclusive manner. For example, where the binding of one ligand to the receptor physically blocks the binding of another ligand to the receptor.

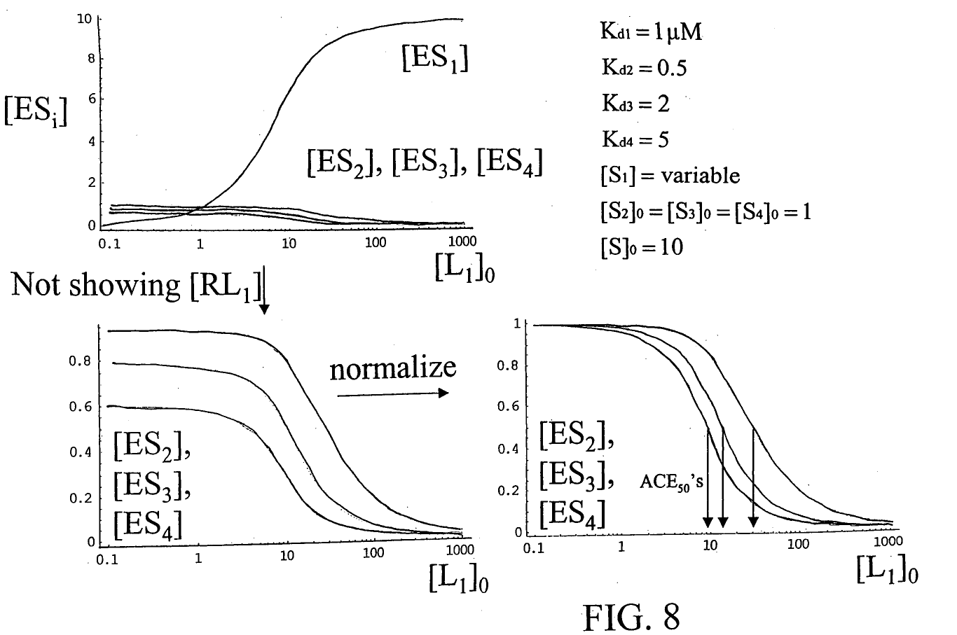

An example of the above described method is depicted in FIG. 8. Normalized plots of the data from FIG. 7 show that the ACE₅₀ values of the ligands in the mixture vary as a function of their K_{d}. Given a set of ACE₅₀ values measured over a range of values of [S₁]₀, the affinity of each component of a mixture of ligands to a protein target can be ranked. Further, the values of ACE₅₀ for each ligand Sᵢ yield quantitative estimates of the individual binding affinities K_{di} by solution of the equation shown in FIG. 6.

The ternary complex model of allosteric binding is shown below. In this model, ligands S₁ and S₂ bind distinct sites on receptor E with dissociation constants K_{d1} and K_{d2} respectively. However, if both ligands bind simultaneously to the receptor, they affect each other's binding constant by a factor α. For example, S₁ binds to E with dissociation constant K_{d1}, but binds to the binary complex E·S₂ to form ternary complex E·S₁·S₂ with dissociation constant α·K_{d1}. Where α>1, allosteric interaction by one of the ligands increases the dissociation constant of the other, resulting in negative cooperativity. Where, α<1, positive cooperativity results, and if α=1, allosteric interaction by one ligand has no affect on the binding of the other.

$$\begin{matrix}E + {S}_{1} & ⇌ & E \cdot {S}_{1} \\ ⥮ {K}_{d2} & & α {K}_{d2} ⥯ \\ E \cdot {S}_{2} & & \\ + {S}_{1} & {⇌}^{α {K}_{d1}} & E \cdot {S}_{1} \cdot {S}_{2}\end{matrix}$$

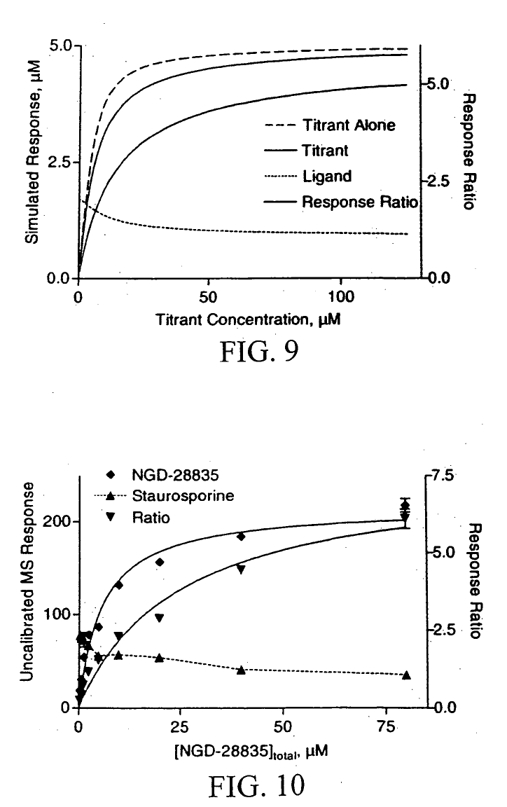

The using a size-exclusion method to separate mixture components does not generally separate the binary protein-ligand complexes from allosterically-bound ternary complexes; for example, when using size-exclusion chromatography, all proteinaceous species co-elute from the SEC stage. As such, the measured recovery of a particular ligand represents the sum of the protein-ligand complexes containing that ligand; e.g., recovered S₁ correlates with the summed concentrations of the products E·S₁ and E·S₁·S₂. FIG. 9 shows the simulated recovery of two allosteric ligands where S₁ with K_{d1}=2.0 μM is titrated into a mixture of receptor E at 5.0 μM concentration plus S₂ with K_{d2}=0.5 μM at 2.0 μM concentration. For cooperativity factor α=10, negative cooperativity between the two sites causes the recovery of S₂ to diminish with increasing titrant concentration. However, its recovery does not diminish to zero as was the case for orthosteric binding competition; rather, the ligand's recovery plateaus because its receptor concentration remains constant (S₂'s binding site is not occupied by titrant) while K_{d2} is increased by the factor α. This has an important and measurable influence on the ratio of the recoveries of the two ligands: rather than linearly increasing with titrant concentration under receptor-limiting conditions, the ratio is hyperbolically curved per the following formula (IX):

$$\frac{E \cdot {S}_{1} + E \cdot {S}_{1} \cdot {S}_{2}}{E \cdot {S}_{2} + E \cdot {S}_{1} \cdot {S}_{2}} = \frac{{[ {S}_{1} ]}_{0} ( {[ {S}_{2} ]}_{0} + α {K}_{d2} )}{{[ {S}_{2} ]}_{0} ( {[ {S}_{1} ]}_{0} + α {K}_{d1} )} \qquad formula ( IX )$$

and is asymptotically bounded at a value of formula (X)

$${Lim}_{{[ {S}_{1} ]}_{0} -> ∞} \frac{{[ {S}_{1} ]}_{0} ( {[ {S}_{2} ]}_{0} + α {K}_{d2} )}{{[ {S}_{2} ]}_{0} ( {[ {S}_{1} ]}_{0} + α {K}_{d1} )} = 1 + \frac{α {K}_{d2}}{{[ {S}_{2} ]}_{0}} \qquad formula ( X )$$

In the case where the negative cooperativity is large, the interactions are indistinguishable from mutually exclusive competitive binding, and the right-hand side of formula (IX) reduces to that of formula (XI):

$${Lim}_{α -> ∞} \frac{{[ {S}_{1} ]}_{0} ( {[ {S}_{2} ]}_{0} + α {K}_{d2} )}{{[ {S}_{2} ]}_{0} ( {[ {S}_{1} ]}_{0} + α {K}_{d1} )} = \frac{{[ {S}_{1} ]}_{0} {K}_{d2}}{{[ {S}_{2} ]}_{0} {K}_{d1}} \qquad formula ( XI )$$

The response ratio data from an ACE₅₀ titration against an allosteric-competitive ligand can be fit to formula (IX) to yield the K_{d} of the titrant and cooperativity factor α given the K_{d} of the ligand, the total ligand concentration [S_{2]}₀, the total receptor concentration [E]₀, and relative MS response calibration factors for the titrant and ligand. Nonlinear regression analysis of the ACE₅₀ response ratio data from FIG. 10 yields a cooperativity factor α=8.3±0.7 and K_{d} value of 3.0±0.3 μM for NGD-28835. This K_{d} value is in good agreement with that of 3.3±1.3 μM measured by an independent titration experiment against the basal kinase.

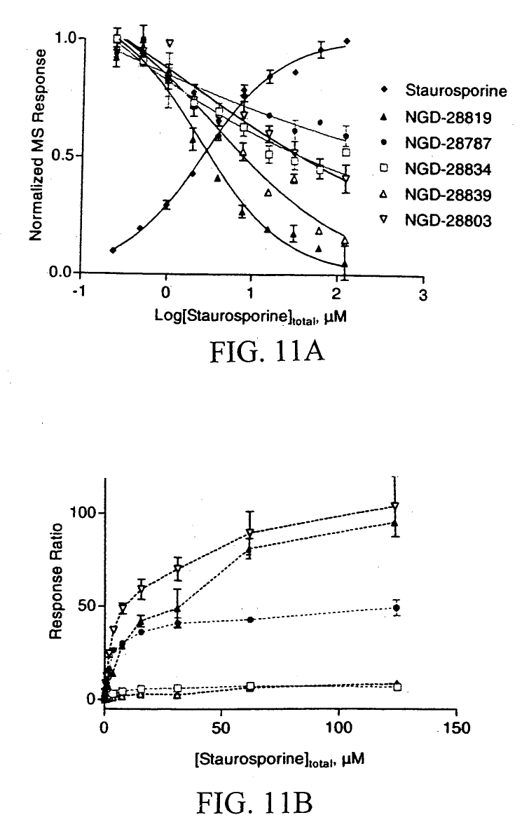

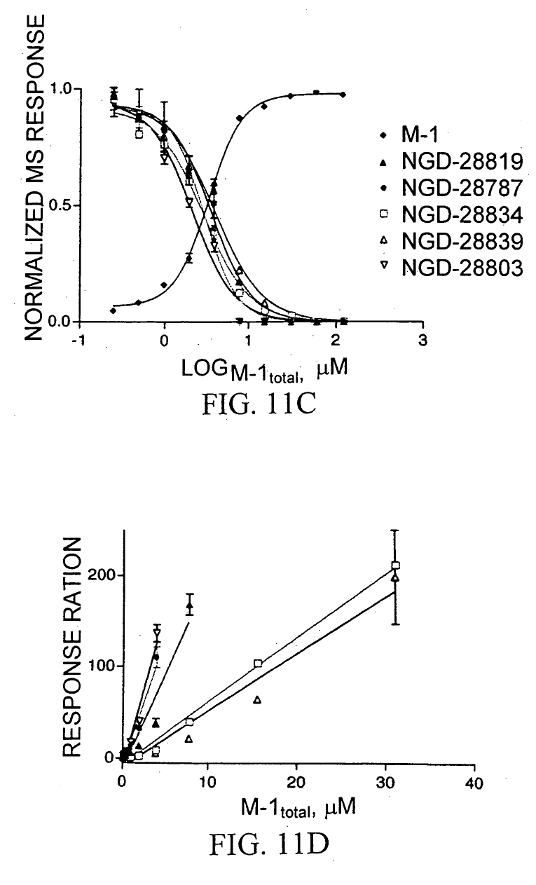

FIGS. 11A-F show the simultaneous binding mode determination for several Akt-1 ligands. Saturation binding by the titrant staurosporine does not quantitatively displace these ligands, and the response ratio curves vs. titrant concentration are asymptotically bounded, suggesting all bind allosterically with respect to the ATP/staurosporine binding site. (FIGS. 11a and 11b) In contrast, competition by the recently reported Akt-1 ligand M-1 (Merck and Company) yields orthosteric binding competition for each component of this same mixture of compounds. These results suggest the M-1 compound and the ligands directly competitive with it bind a site distinct from the ATP/staurosporine binding site. (FIGS. 11c and 1d)

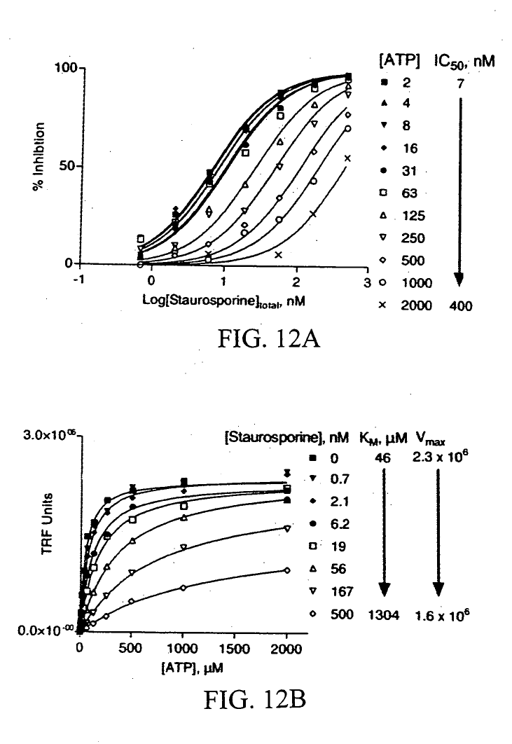

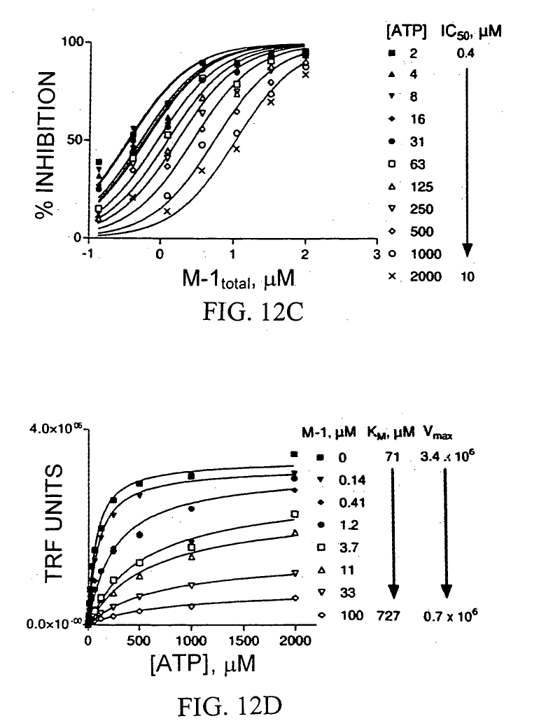

To independently evaluate this conclusion, inhibition of Akt-1 kinase activity by M-1 and staurosporine was examined at varying ATP concentrations as shown in FIGS. 12a-d. Staurosporine binds the ATP-binding site and, consistent with this implication, increasing ATP concentrations increase the measured IC₅₀ for the nanomolar inhibitor staurosporine by greater than 50-fold. Without wishing to be bound by theory, the increase in staurosporine IC₅₀ with increasing ATP concentration is primarily due to an increase in ATP Kₘ. The effect of staurosporine on Vₘₐₓ for this reaction is modest, even at high ATP concentration. These results confirm staurosporine is an orthosteric competitor of ATP. (FIGS. 12a and 12b)

Though the IC₅₀ of M-1 is only micromolar, its IC₅₀ increases by a smaller factor over the same ATP concentration range than the nanomolar IC₅₀ of staurosporine. The increase in the IC₅₀ of M-1 with increasing ATP concentration is due to a five-fold decrease in Vₘₐₓ coupled with a smaller increase in Kₘ than is observed for staurosporine. These results show that M-1 is a mixed, non-competitive inhibitor of Akt-1, indicating a biochemical mechanism of action that includes both ATP displacement (increased ATP Kₘ) and slowing of Akt-1 kinase activity (decreased Vₘₐₓ), and confirming that M-1 binds at a site topographically distinct from the ATP- and staurosporine-binding pocket of Akt-1. (FIGS. 12c and 12d) This conclusion is consistent with the ACE₅₀ results, which suggest M-1 and staurosporine simultaneously bind Akt-1, and illustrates how the ACE₅₀ technique may be used to evaluate orthosteric and allosteric binding mechanisms of ligand mixtures.

In addition to illuminating details of their Akt-1 binding mechanism, the M-1 titration experiment also yields an affinity ranking for the mixture components. NGD-28839 has the highest ACE₅₀ value, indicating it is the highest affinity ligand in this test mixture as it requires the highest concentration of titrant M-1 for displacement. NGD-28839 shows the best biochemical activity of the mixture components, yielding 44±8% inhibition of Akt-1 kinase activity at 50 μM concentration. The ACE₅₀ value for NGD-28839 in FIG. 5 is 4.1 μM (95% c.i. 3.4 to 5.0 μM), corresponding to a K_{d} of 3.5±0.7 μM (95% c.i. 1.5 to 10.2 μM) given a K_{d} of 0.3±0.1 for M-1. The other ligands in this mixture show weaker inhibition of Akt-1 activity than NGD-28839, consistent with their lower affinities as indicated by ACE₅₀ titration.

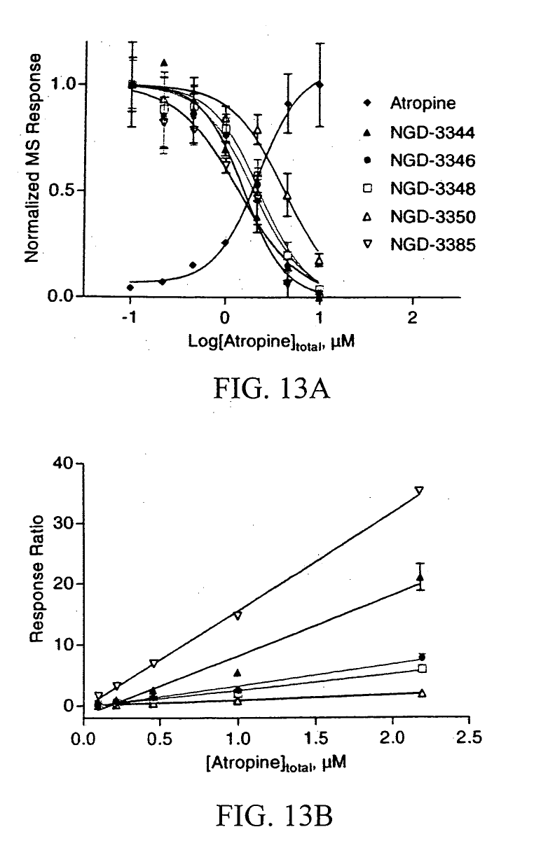

Affinity-ranking and affinity optimization in compound mixtures using the ACE₅₀ method is further demonstrated in FIGS. 13a-b using a small library of ligands to the muscarinic acetylcholine receptor M₂, a GPCR. This ligand pool includes chemotype representatives of several compounds discovered through AS-MS-based high-throughout screening of mass-encoded libraries, as well as some structural analogs of NGD-3346. The known M₂ ligand Atropine was used as the titrant against 2.0 μM M₂ in the presence of 0.5 μM per component compound pool. The response ratio plots are linear, (FIG. 13b) indicating that all the ligands examined are orthosterically competitive with Atropine. Consistent with this result, independent biochemical assays show that all the ligand tested, like Atropine, are antagonists of M₂. The ACE₅₀ curves indicate clear differences in affinity, with NGD-3350 exhibiting a 10-fold higher affinity than its structural congeners NGD-3348 and NGD-3346. (FIG. 13a) Independent biochemical activity measurements confirm this result: NGD-3350 exhibits an IC₅₀ of 1.6 μM in a cell-based cAMP assay and an IC₅₀ of 9.6 μM in a tissue-based assay for M₂ antagonism. Though the remaining compounds all exhibit modest M₂ antagonist activity in the cAMP assay, only NGD-3350 shows significant activity in tissue. These results highlight the utility of the ACE₅₀ method to simultaneously rank-order compounds by affinity, particularly for mixtures of structural analogs synthesized by combinatorial chemistry techniques, and identify those compounds with improved affinity relative to a progenitor, e.g. the improved affinity of NGD-3350 relative to its parent NGD-3346.

The equilibrium concentrations of the products and reactants of a single-site equilibrium binding are described by the equilibrium dissociation constant K_{d}, which can also be expressed as the ratio of the dissociation rate constant k₂ to the association rate constant k₁ as shown below in formula (XII).

$${K}_{d} = \frac{{k}_{2}}{{k}_{1}} = \frac{[ E ] [ S ]}{[ ES ]} = \frac{[ {E}_{0} - ES ] [ {S}_{0} - ES ]}{[ ES ]} \qquad formula ( XII )$$

It can be seen from this equation that for a given association rate constant k₁, a lower value of dissociation rate constant k₂ will yield a smaller value of K_{d} and hence a higher equilibrium concentration of the desired protein-ligand complex.

The overall rate of change in the concentration of receptor-ligand complex E·S (or, more simply, ES) with time, dES/dt, is the difference between its rate of formation and its rate of depletion (see formula (XIII).

$$\frac{ⅆ [ ES ]}{ⅆ t} = {k}_{1} [ E ] [ S ] - {k}_{2} [ ES ] \qquad formula ( XIII )$$

The rate of formation of ES depends upon the association rate constant k₁, the total concentration of receptor E₀ and the total concentration ligand S₀, while the rate of dissociation of ES depends on the concentration of ES and upon the dissociation rate constant k₂. Therefore, dES/dt can be modeled using the formula (XIV) below for single-site, reversible binding between a receptor and a single ligand.

$$\frac{ⅆ [ ES ]}{ⅆ t} = {k}_{1} [ {E}_{0} - ES ] [ {S}_{0} - ES ] - {k}_{2} [ ES ] \qquad formula ( XIV )$$

It should also be noted that when dES/dt is zero, (by definition, when the system is at equilibrium,) formula (XIV) reduces to the equilibrium expression in formula (XII).

In analogy to the single ligand-single site equilibrium described above, competitive binding between a ligand, S, and an inhibitor ligand, I, is described below. As shown in formula (XV),

$$EI {{⇌}^{{k}_{i2}}}_{ {k}_{i1 }} E + I + S {{⇌}^{{k}_{s1}}}_{ {k}_{s2 }} ES \qquad formula ( XV )$$

free receptor, E, can react either with free ligand, S, to form the complex ES or react with free inhibitor, I, to form complex EI. The overall rate of change in the concentration of protein-ligand complexes ES and EI are described by formulae (XVI) and (XVII), respectively.

$$\begin{matrix}\frac{ⅆ [ ES ]}{ⅆ t} = {k}_{s1} [ {E}_{0} - ES - EI ] [ {S}_{0} - ES ] - {k}_{2} [ ES ] & formula ( XVI ) \\ \frac{ⅆ [ EI ]}{ⅆ t} = {k}_{i1} [ {E}_{0} - ES - EI ] [ {I}_{0} - EI ] - {k}_{2} [ EI ] & formula ( XVII )\end{matrix}$$

The behavior of the system can be modeled using a simultaneous numerical solution of these two equations given values for the system parameters E₀, total receptor concentration; S₀ and I₀, total ligand and total inhibitor concentrations; kₛ₁, kₛ₂, kᵢ₁, and kᵢ₂, the rates of association and dissociation for the interacting components of the mixture, and initial concentrations for ES and EI.

Simultaneous solution of formulas (XVI) and (XVII) with the initial value of ES not equal to zero allows one to model the behavior of the system with time upon sudden addition of an excess of inhibitor. Where a large excess of inhibitor I is added to the solution of ES, kₛ₁, the association rate of E and S can be estimated to be zero. This is because when a receptor-ligand complex ES dissociates, the receptor will then be bound to inhibitor I, since I is present in a large excess, leaving only the dissociation available to the ES complex and closing off the pathway for E and S to reassociate. Thus, the large excess of inhibitor I, where S and I bind to E competitively, occupy substantially all of the binding sites of E. Formula (XVIII), depicts a solution to formula (XVI), where a large excess of inhibitor I is added to a mixture of ES.
[ES]=[ES]ₜ₌₀e^{-ks2·t} formula (XVIII)

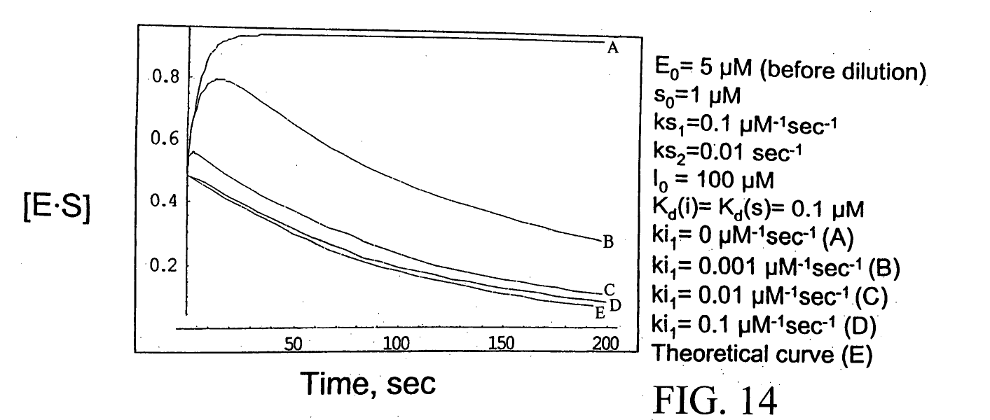

FIGS. 14-17 are examples of mathematical modeling experiments which simulate the response for a receptor-ligand complex vs. time when subjected to changes in inhibitor concentration and variation in other parameters as indicated, including association and dissociation rates for the ligand S and inhibitor I. Solving the equation provides both the kinetic dissociation rate k₂ and the half-life of the receptor ligand complex. The relation of kₛ₂ to t_{1/2} is shown in formula (XIX).
Half-life t_{1/2}=0.693÷kₛ₂ formula (XIX)

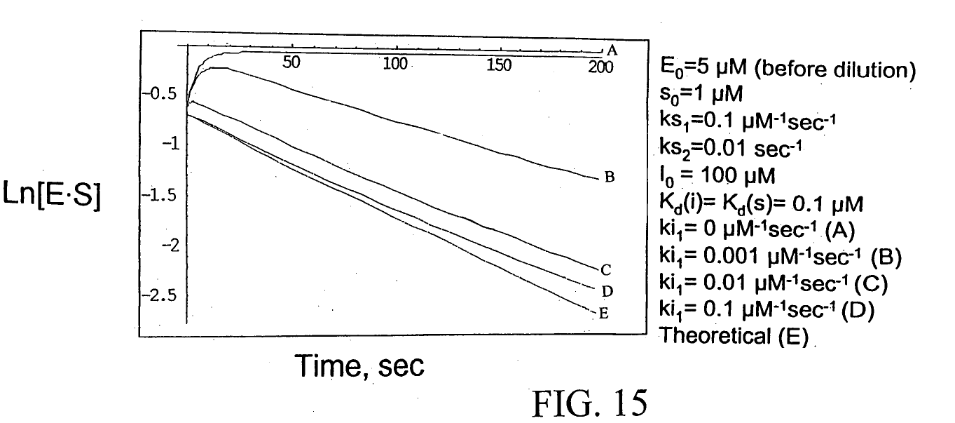

FIG. 14 is a mathematical model of a system consisting of 5 μM receptor and 1 μM ligand with typical association and dissociation rates to which has been added a large excess of inhibitor by 1:1 dilution of the original, equilibrated receptor-ligand mixture with inhibitor while keeping the total ligand concentration constant. As mentioned above, the rate at which the inhibitor competes with the ligand for the receptor depends upon both the dissociation rate of the receptor-ligand complex and the rate of association of the inhibitor and receptor; the results in FIG. 14 are shown for inhibitors of varying association rate. Dilution of the receptor with a non-associating inhibitor (kᵢ₁=0) containing an equal total concentration of ligand as the equilibrated receptor-ligand mixture causes the total receptor and receptor-ligand complex concentrations to initially drop to 50% of their original value, then (in the absence of active inhibitor) the system restores itself to a new equilibrium. However, in the presence of an excess of an associating inhibitor (kᵢ₁≠0), any free receptor is rapidly quenched by the inhibitor so no receptor-ligand complex can re-form. Also, as soon as any receptor-ligand complex spontaneously dissociates, the rate of which depends upon kₛ₂, the freed receptor is quenched by the excess of inhibitor. Therefore, the measurable concentration of the receptor-ligand complex will diminish with time after addition of an excess of inhibitor. It can be seen that even with a very slow-binding inhibitor (kᵢ₁=0.001 μM⁻¹sec⁻¹) the slope of the decay curve approaches that of the theoretical decay curve [ES]₀e^{-ks2·t} (formula XVIII) that would result from pure dissociation kinetics. As the decay follows an exponential function, the similarity of the experimental decay curve and the theoretical one is even more apparent when the plots are compared in log space, as shown in FIG. 15.

For example, when an excess of I is introduced to a mixture of ligands and receptor, the relative binding kinetics of the plurality of ligands can be determined in a relative sense. Because it can be assumed, in the case of a sufficient excess of I, that a ligand, once becoming unbound from a receptor will not become rebound to a receptor, a ligand whose signal response decreases the fastest in a mixture of ligands can be determined to have the highest off rate of the ligands.

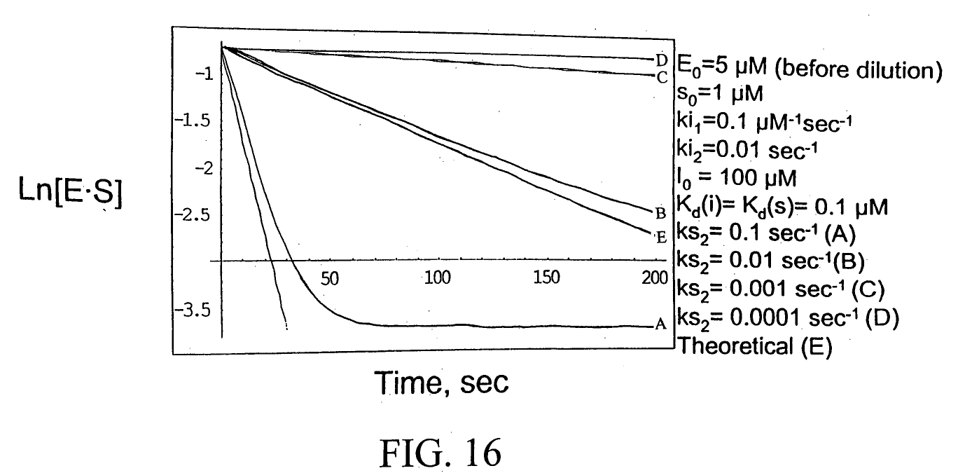

An example of this method is also depicted in FIG. 16. This figure demonstrates mathematically a system consisting of 5 μM protein and 1 μM ligand with a typical receptor-ligand association rate and varying receptor-ligand dissociation rates to which has been added a large excess of inhibitor by 1:1 dilution of the original, equilibrated receptor-ligand mixture with an excess of inhibitor while keeping the total ligand concentration constant. For each ligand dissociation rate modeled, the theoretical decay curve [E·Sub]₀e^{-ksub2·t} (formula (XVIII)) that would result from pure dissociation kinetics is also shown. The model indicates that the receptor-ligand complex's rate of diminution closely correlates with the absolute dissociation rate.

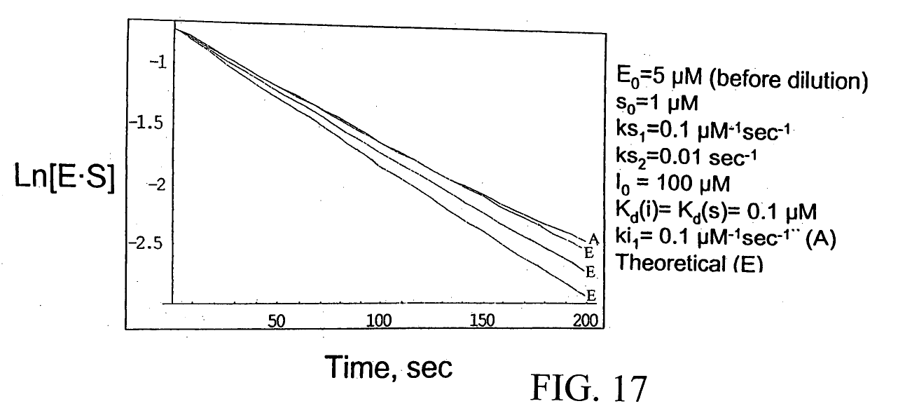

The exact degree of correlation of the rate of decay of the receptor-ligand complex in a modeled competitive binding experiment and the theoretical decay curve expected from measurement of the pure dissociation rate is evaluated in FIG. 17. The modeled decay curve is shown for kₛ₁=0.01 sec⁻¹ and theoretical curves are shown for dissociation rates ±0% of this value. The results indicate that the measured dissociation rate is within ˜10% of the actual value, a very good approximation of the actual dissociation rate given the simplicity of the experimental method.

Since the experimentally measured decay curve closely matches the exponential decay curve expected for first-order dissociation kinetics, the experimental data can be fit to the pure exponential decay function using available curve-fitting algorithms such as those supplied with Microsoft Excel® to extract dissociation rate and half-life information about each ligand. Given a set of protein-ligand complex concentration values measured versus time by consecutive experiments performed after addition of an excess of a competitive inhibitor to an equilibrated mixture of a protein and a ligand or ligands of interest, the dissociation rate of each ligand can be quantitatively estimated and the relative rates of multiple ligands in a mixture can be qualitatively ranked.

The term “noncompetitive ligand” refers to a ligand that binds to a receptor at a second site other than the active site. The binding of the noncompetitive ligand can occur simultaneously with another ligand (e.g., at the catalytic site of an enzyme) or can bind the receptor alone yet still affect the response of the receptor. In some cases a noncompetitive ligand (e.g., a noncompetitive ligand for an enzyme) can act by decreasing the effectiveness of the receptor (e.g., decreasing the effectiveness of the enzyme, or altering receptor conformation, including active site conformation).

Where a ligand is a noncompetitive ligand, the introduction of an excess of inhibitor I, will not act to prohibit the reassociation of the noncompetitive ligand once it has dissociated from the receptor-ligand complex. Thus, the signal response of a noncompetitive ligand over time, after the addition of an excess of inhibitor, will remain constant.

Yet another method of comparing ligands and identifying ligands having desirable binding characteristics includes the comparison of ligand binding under “receptor-limiting” and “receptor in excess” conditions. Simultaneous solution of the formula (I) below

$${k}_{di} = \frac{( {[ E ]}_{0} - {\sum }_{i}^{} [ {ES}_{i} ] ) ( {[ {S}_{i} ]}_{0} - [ {ES}_{i} ] )}{[ {ES}_{i} ]} \qquad formula ( I )$$

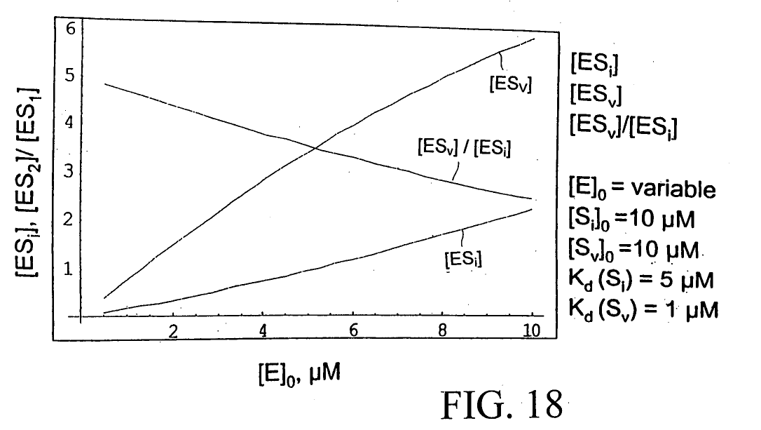

for a theoretical two-component mixture at a fixed concentration of a “initial” (e.g., known) ligand Sᵢ and of a second “varied” (e.g., related or improved) ligand Sv over a range of receptor concentrations yields a graphical representation of receptor-ligand complex concentrations [ESᵢ] and [ESᵥ] shown in FIG. 18 for the conditions therein annotated. As the total receptor concentration [E]₀ is increased from [E]₀<[Sᵢ]₀ to [E]₀>[Sᵢ]₀ the experimental conditions are described as proceeding from “receptor-limiting” to “receptor in excess.” The ratio of the receptor-ligand complex for the tighter ligand to that of the weaker ligand increases as the experiment approaches receptor-limiting conditions, indicating that the tighter ligand outcompetes the weaker ligand for the limited amount of receptor present. Thus, ranking of the binding affinities of the ligands in a mass-encoded mixture is accomplished by contacting a mixture of ligands, including an initial (e.g., an original lead) compound, with the receptor under receptor-limiting conditions and, separately, receptor-excess conditions; quantifying the resulting receptor-ligand complexes by affinity selection-mass spectrometry; and comparing the ratios of the signal responses of each ligand; where the ratios are the signal responses of 1) the ligands in the absence of receptor to the signal responses of each ligand under receptor excess conditions and 2) the signal responses of the ligands in the absence of receptor to the signal responses under receptor-limiting conditions.

The methods described herein can be performed by separating components of mixtures, using size-exclusion chromatography, which separates unbound ligands from receptors and receptor bound ligands, followed by liquid chromatography. The liquid chromatography can be performed under conditions that dissociate the receptor from the ligand. The dissociated receptor can then be passed through a mass spectrometer to provide a signal response.

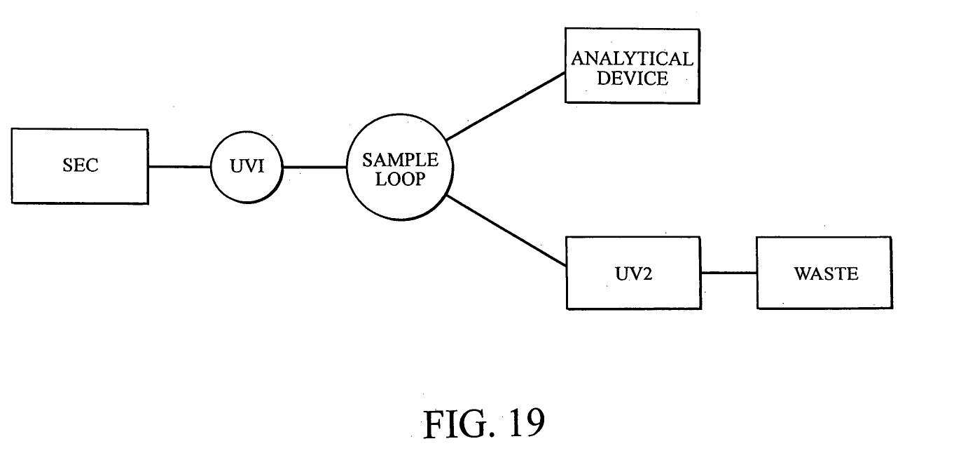

FIG. 19 shows, from left to right, the path a ligand follows as a receptor-ligand complex is identified within a sample in a method described herein. A sample that includes receptor, ligands, and receptor-ligand complexes is passed through a size-exclusion-column (SEC) where the eluant is monitored with a UV detector (UV1). The sample then enters a sample loop where it is transferred either to a second analytical device, or diverted to an outflow (e.g., waste), which can be monitored by a second UV detector (UV2) as depicted in FIG. 19.

Many screening and analytical techniques rely upon light absorbance detectors (e.g. UV, fluorescence) to determine the absence or presence of a receptor in liquid samples. UV detectors measure the absorbance of UV light by material passed through the detector. Absorbance is typically plotted versus time on a chart. Material is detected after it passes through the size-exclusion chromatography apparatus, and an output peak is generated on a chart or monitor. The size of the peak (i.e., volume) corresponds to the extent of absorbance of a sample or material at a particular wavelength.

Before subjecting sample material to a UV detector, receptor-ligand complexes are first separated from unbound ligand and other impurities using size-exclusion chromatography. Each separated component of the sample registers a peak in the UV trace, which is created on a chart. Molecular species with large physical size are the first species to elute from size-exclusion chromatography and register the early UV peaks. For example, in the case of a sample having a receptor that is a protein and a ligand that is a small molecule where some of the receptor and ligand form a receptor-ligand complex, the receptor and receptor-ligand complex will co-elute at the front of the eluant stream, which is due to the relatively large size of the protein compared to the much smaller size of the small molecule ligand.

The term “size-exclusion-chromatography” (SEC) refers to the use of porous particles to separate molecules of different sizes. It is generally used to separate biological molecules, and to determine molecular weights and molecular weight distributions of polymers. Generally, molecules that are smaller than the pore size can enter the particles and therefore have a longer path and longer transit time than larger molecules that cannot enter the particles. Molecules larger than the pore size cannot enter the pores and elute together as the first peak in the chromatogram. This condition is called total exclusion. Molecules that can enter the pores will have an average residence time in the particles that depends on the molecule's size and shape. Different molecules therefore have different total transit times through the column. Molecules that are smaller than the pore size can enter all pores, and have the longest residence time on the column and elute together as the last peak in the chromatogram.

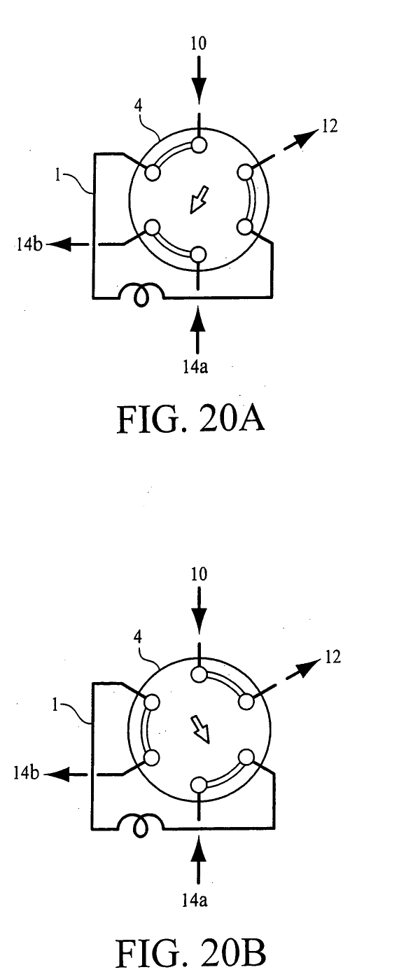

Sample exists the SEC through an eluant stream (“SEC eluant”) that passes through a UV detector. As depicted in FIG. 20A, SEC eluant 10 is passed through a sample loop 1, (e.g., a 1, 5, 10, 15, 20, 25, 50, 100, 150, 200, 250, or 300 μl sample loop) mounted on a high-speed 2-position 6-port selection valve 4 and flows into outflow eluant 12 until the UV detector (not shown) detects a peak that includes a receptor-ligand complex. This initial position of the sample loop 1 and the valve 4 is referred to as the “load” position.

Upon detection of a receptor-ligand complex, a controller connected to a computer (not shown) triggers a timer, which is set for a predetermined amount of time that is sufficient to fill the sample loop 1 with receptor-ligand complex (e.g., 2-5 seconds). Once the sample loop 1 is filled with receptor-ligand complex, the controller rotates the sample loop 1 in the 2-position 6-port selection valve 4 to the position depicted in FIG. 20B. Accordingly, the filled sample loop includes a representative view of all receptor-ligand complexes in the sample.

This position is referred to as the “inject” position. As seen in FIG. 20B, the SEC eluant 10 and outflow eluant 12 are no longer connected to the sample loop 1, but rather, the sample loop 1 is connected to a different eluant stream, which is connected to an analytical device. Analytical device eluant 14a (e.g., solvent) flows into and through the sample loop 1 and exits the sample loop 1 as sample eluant 14b, where the sample eluant 14b flows into the analytical device (not shown) for identification (e.g., mass spectrometry analysis).

The analytical equipment can be scaled to handle samples of various sizes, including small or micro samples. Sample parameters for a 15 μl sample loop are as follows: a connecting tube from the SEC to the multiport valve 4 is 35 cm×75 μm; the sample loop is 84 cm×150 μm; the connecting tube for the analytical device eluant 14a to the sample loop is 40 cm×20 μm; the connecting tube from the sample loop to the analytical device (i.e., sample eluant) 14b is 50 cm×100 μm; and the connecting tube from the sample loop to the outflow eluant 12 is 45 cm×75 μm. The sample loop used to collect eluant after the SEC is made out of capillary tubing coated with polymer. The capillary tubing of desired length (i.e., for the desired volume) is then rolled into a loop to reduce the space it occupies.

The controller can be operated manually, e.g., by a person observing UV peak detection and a timer. Alternatively, the controller can be connected to a computer and operated with a software program. For example the software program can begin a timer upon detection of a receptor-ligand complex by the UV detector, wherein at a predetermined time, the controller rotates the sample loop 1 from the load position depicted in FIG. 20A to the inject position depicted in FIG. 20B.

In some instances, the software used to operate the controller is built around a National Instruments data acquisition (DAQ) board equipped with both digital and analog inputs. A contact closure from the size-exclusion chromatography system is connected to one of the digital inputs. When a sample injection is made, the contact closure is closed and the digital input is triggered. This notifies the controller that a sample run has initiated.

The methods described herein can use two UV detectors, where each detector outputs an analog signal ranging from −1V to +1V that is relative to the absorbance level of the sample. For example, a first UV detector can be positioned between the SEC column, (i.e., in the stream of the SEC eluant 10) and the multiport valve 4. A second UV detector can be positioned in the stream of outflow eluant 12. The signals are connected to two of the controller's analog inputs. The signals are converted to digital using an on board 12-bit analog to digital (A/D) converter. Using the digital signals, the controller can then track peaks passing through the UV detector.

Due to the nature of size exclusion chromatography, the first peak exiting the UV detector is generally the peak containing the receptor-ligand complex and free receptor. The controller waits for the first rise in signal and registers it as a peak. The capture of the peak (i.e., the sample) in the sample loop is based on a timer initiated with the detection of the peak.

The flow rate of the pump pushing solvent through the UV detector can be constant at 2 mL/min. At that flow rate, it takes 3.3 seconds to fill a 100 μL sample loop. Thus the time delay required to fill the sample loop 1 with detected material after peak identification is approximately 3.3 seconds. However, due to possible signal delays from the UV detector to the controller, the time delay is determined experimentally for each system configuration (i.e., the system is calibrated, and is recalibrated for each variation in the system). Because the volume and flow rate do not change from sample to sample, any variation in travel time from the UV detector to the sample loop is due solely to any instability in the flow rate generated by the solvent pump. It is therefore implicit that any change in plumbing requires re-evaluation of the time delay (e.g., where the system is altered or any component is replaced or adjusted).

The two UV detectors can be used to capture data before and after the rotation of the multiport valve, which diverts the sample captured in the sample loop to an analytical device such as an LC-MS. Plots of the data from the two UV detectors visually illustrate the sample capture for every sample. One detector tracks the receptor-ligand complex and is used by the controller to time the rotation of the sample loop and valve. The second detector is connected to the flow of sample that is diverted away from the analytical device. The trace from this detector reveals what was not captured by the sample loop. A comparison of the two traces indicates the efficiency of peak capture.

In order to improve the accuracy of sample detection in the UV device, an algorithm is used to collect and process data and determine the presence of a peak (i.e., presence of a receptor-ligand complex). The DAQ board collects data at a maximum frequency of 20 kHz. Raw data, however, can often be too unstable to use for peak identification. For example, samples of low protein concentration can be missed due to signal noise. Accordingly, an algorithm can be used to smooth the data and to provide a reliable data output.

The presently used algorithm collects and averages sets of raw data at preset time intervals. These averaged sets are then further processed to create a moving average of data which is tracked by the controller for peaks. For example, at a sampling rate of 10 kHz (10,000 data points per second), sets of 500 points of raw data are continuously collected and averaged every 50 ms. The first seven sets (each of 500 points of raw data) are designated A, B, C, D, E, F, and G. Sets A through E are averaged to create data point 1. Sets B through F are averaged to create data point 2. Sets C through G are averaged to create data point 3, and so on. The data points are plotted by the control system and checked for peaks.

The criteria for peak detection is simply to locate three consecutive data points that increase in value over an initial threshold. The threshold is determined by tracking data points over the first 2 seconds of data capture during which time only sample buffer is passing through the UV detector. The highest data point is set as the threshold. This eliminates hits on false positive peaks by accounting for the UV absorbance of the buffer as well as variances in signal due to line noise. The threshold can optionally be raised an additional preset amount by a user.

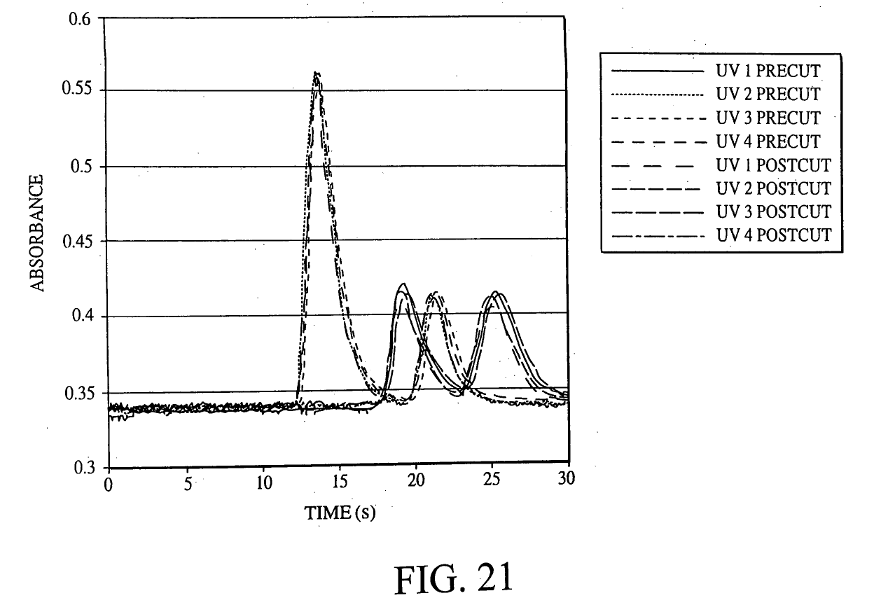

Representative results of the separation and identification of Human Serum Albumin (HSA) and ligand from a sample on a 50 mm size-exclusion chromatography column at a 2 mL/min flow rate using phosphate buffer are displayed in FIG. 21. The relative heights of the UV1 and UV2 traces indicate that each peak is cut by the same amount (˜60% relative peak area) in a reproducible manner. Although the retention times of the peaks are virtually identical, this is not necessary for proper system operation. Peak detection and cutting happens dynamically relative to the location of the peak so retention time is irrelevant.

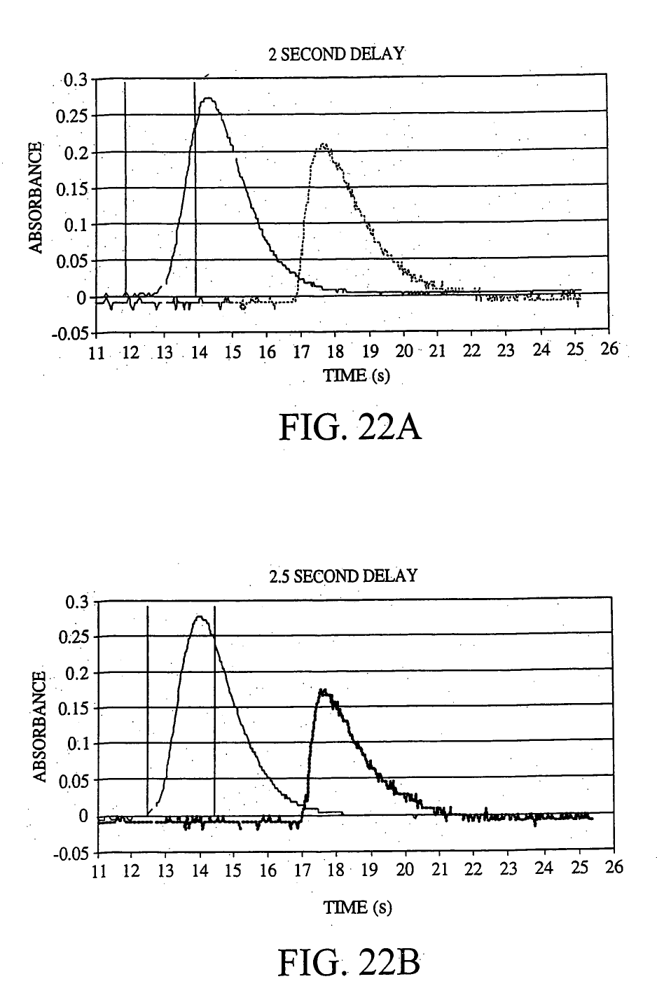

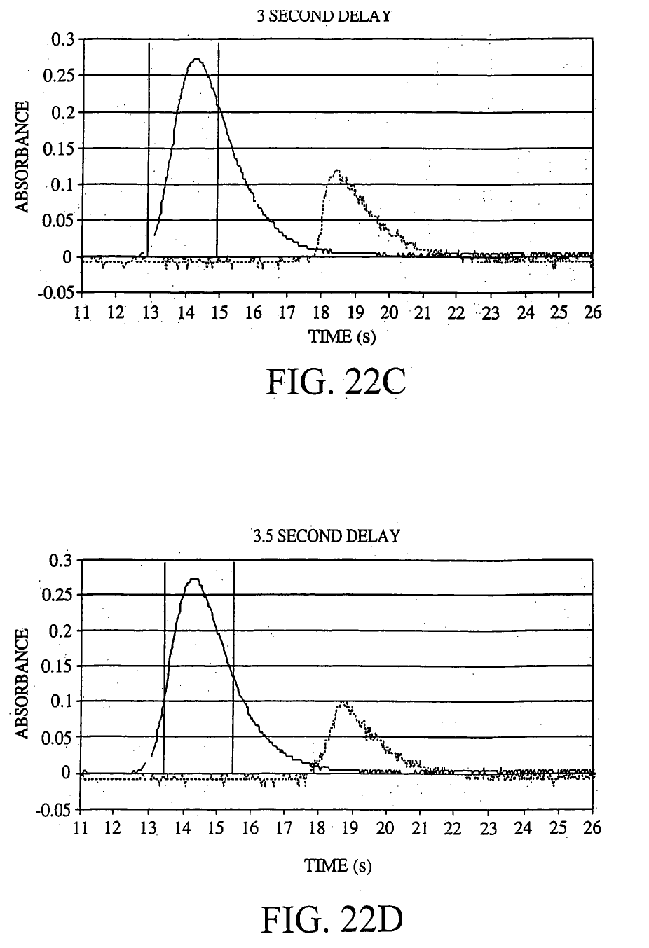

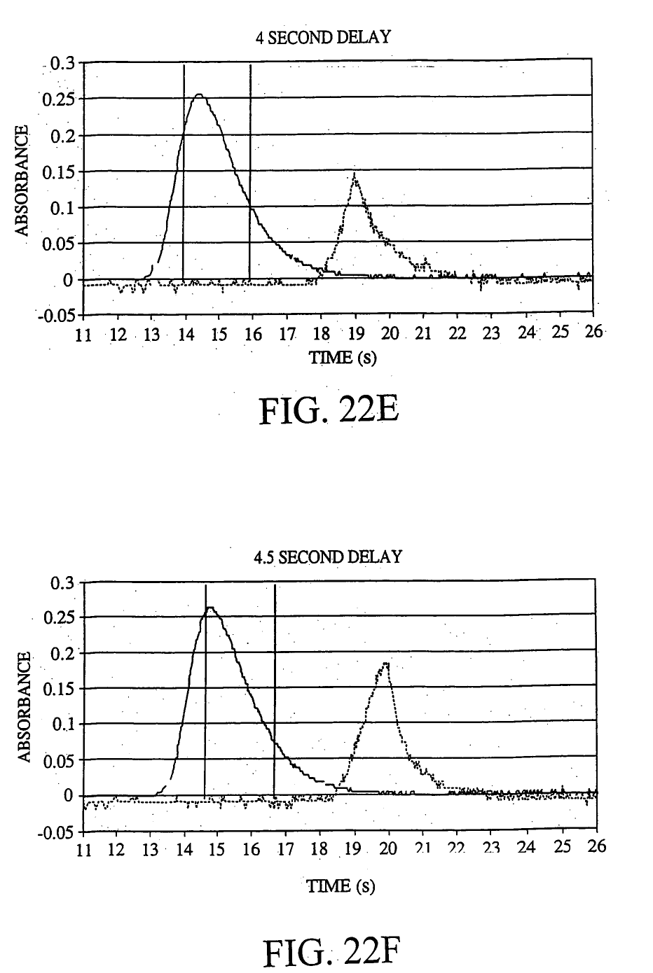

Changing the time of the peak cut is easily modified and the results of doing so HSA system are shown in FIGS. 22A-22F. Accordingly, when using the methods described, the timing of the change from the load position to the inject position of the injection port valve can be adjusted depending on what portion of the peak the user wants to collect. FIG. 22A depicts the portion of the peak captured in the sample loop after 2 seconds. FIG. 22B depicts the portion of the peak captured in the sample loop after 2.5 seconds. FIGS. 22C-22F depict the portion of the peak captured in the sample loop after 3.0 seconds, 3.5 seconds, 4.0 seconds, and 4.5 seconds, respectively. As can be seen in FIGS. 22A-22F, different portions of the peak (e.g., different portions of the detected receptor-ligand complex) can be captured in the sample loop by varying the delay time from detection to rotation of the sample loop with the controller. The vertical lines on the plots approximate the 100 μL portion of the sample captured by the sample loop. In the data shown, 3 seconds (See FIG. 22C) is the optimal value for delay time.

Although the expected sample travel time is calculated, variances in valve dead volumes and UV detector flow cells from rated values necessitates calibration. Lag time in the detector output is also a factor that need be accounted for. In each instance, a calibration run of a sample can determine the variance in travel time due to these factors.

Increasing the size of the sample loop can allow for a greater portion of the sample peak to be captured. However, the front portion of the peak is often the most desirable portion as it not only contains the receptor-ligand complex, but also is the least likely to contain any breakthrough unbound ligand. Breakthrough is the phenomenon by which small molecules are observed to pass through a size-exclusion chromatography column with apparent masses much greater than their real masses, thereby appearing as unresolved peaks with the protein signals.

The term “signal response” refers to an output for an analytical device or detector (e.g., a mass spectrometer, UV, fluorescence, HPLC, NMR, IR, etc.) that correlates in a measurable way with the amount of material of interest present in the analytical device. The signal response of a particular material is dependent on a number of factors relating to the material itself (e.g., UV activity, ionization potential, etc.) as well as factors relating to the analytical device or detector (e.g., sensitivity). The signal response for a material can be linear in relation to the amount of material. However, it is not necessary for a signal response to be linear in relation to the amount of material, rather it is only necessary that the signal response be dependent in a measurable way on the amount of material being assessed. For example, the signal response can have a logarithmic or exponential relation to the amount of material being analyzed in the analytical device or detector.

The term “calibrating” refers to the determination, by measurement or comparison with a standard, of the correct value of each scale reading on a meter or other measuring instrument. For example, an analytical or measuring instrument (e.g., a mass spectrometer) can be calibrated by measuring or determining a plurality of values of an analyte (e.g., concentrations of ligands) whose true values are known. The measured values may be “absolute,” that is a true measure, or may be “relative,” that is correlates to the measured values such that the relative values are indicative of the measured values over the range of values.

The term “mass spectrometer” refers to an analytical device that uses the difference in mass-to-charge ratio (m/e) of ionized atoms or molecules to separate them from each other. Mass spectrometry is therefore useful for quantitation of atoms or molecules and also for determining chemical and structural information about molecules. Molecules have distinctive fragmentation patterns that provide structural information to identify structural components. The general operation of a mass spectrometer is: (a) create gas-phase ions; (b) separate the ions in space or time based on their mass-to-charge ratio, and (c) measure the quantity of ions of each mass-to-charge ratio. The ion separation power of a mass spectrometer is described by its resolution.

There are many ionization sources known in the art, for example, electrospray ionization (ESI), electron ionization (EI), fast atom bombardment ionization (FAB), matrix-assisted laser desorption (MALDI), electron-capture (sometimes called negative ion chemical ionization or NICI), and atmospheric pressure chemical ionization (ApCI). The ions produced in any of the ionization methods above are passed through a mass separator, typically a magnetic field, a quadrupole electromagnet, or a time-of-flight mass separator so that the mass of the ions may be distinguished and the number of ions at each mass level quantified.

Mass spectrometry (MS) is a widely used technique for the characterization and identification of molecules, both in organic and inorganic chemistry. MS provides molecular weight information about a molecule. The molecular weight of a molecule is a crucial piece of information in the identification of a particular molecule in a mixture of molecules. MS analysis can be used, for example, in drug development and manufacture, pollution control analysis, and chemical quality control.

The term “ligand” refers to a molecule that associates with a receptor (e.g., interacts in a covalent or non-covalent manner). In some cases, the binding of the ligand to the receptor can have a biological effect (e.g., agonism or antagonism). For example, the ligand can be a polypeptide (e.g., a protein) binding to a biomolecule (e.g., DNA molecule) wherein the binding of the protein to the DNA initiates mRNA synthesis. The ligand can also be an organic molecule (e.g., a pharmaceutical compound) bound to an enzyme (e.g., HIV protease) wherein the binding of the organic molecule to the enzyme inhibits enzymatic activity.

The term “variant ligand” refers to a molecule that is related to an initial ligand, but is different from that ligand. For example a variant ligand can have the same core structure of an initial ligand but have one or more differing peripheral groups attached to the core structure. The differing groups can vary slightly, for example changing a methyl to an ethyl group. Alternatively, the differing groups can vary more significantly, for example, changing an amide group to an aromatic group. Variant ligands can also include ligands having changes from the core structure of an initial ligand, for example changing a phenyl ring to a pyridyl ring, etc.

The term “i” is an integer between 1 and the number of ligands (i.e., ligands having distinct a distinct structure) in a mixture of receptor and ligands, wherein “i” represents an individual ligand within the mixture.

The term “organic molecule” refers to a non-peptidic compound wherein the molecule includes carbon and hydrogen, and can also include additional elements such as nitrogen, oxygen, phosphorus, halogens, or sulfur (e.g., an pharmaceutical compound). Pharmaceutically acceptable salts (e.g., maleic, hydrochloric, hydrobromic, phosphoric, acetic, fumaric, salicylic, citric, lactic, mandelic, tartaric and methanesulfonic) are also encompassed within the meaning of the term “organic molecule.”

The term “inhibitor” refers to a molecule that associates with a receptor and in so associating with the receptor, affects function of the receptor or interferes with association of the receptor with another ligand. In some instances, the inhibitor associates in a manner that is competitive with a ligand.

The term “receptor” refers to a molecule upon association with a second molecule, enables or initiates and effect (e.g., biological activity or detectable signal). For example, a receptor can be a protein that binds a specific extracellular signal molecule (e.g., a ligand) and initiates a response in the cell. Examples of cell-surface receptors include the acetylcholine receptor and the insulin receptor. Examples of intracellular receptors include hormones, which can bind ligands that diffuse into the cell across the plasma membrane. Other examples of receptors include: polypeptides, proteins, enzymes, ribozymes, RNA, DNA, and biomolecular mimics.

The term “biomolecule” refers to a molecule having an effect on biological activity (e.g., metabolism, antagonism, agonism, signaling, or transcription). While a biomolecule can be found in a living organism, the term biomolecule is not limited to naturally occurring biomolecules and includes synthetic versions of naturally occurring biomolecules as well as fragments and modifications thereof. Examples of biomolecules include: polypeptides, proteins, enzymes, ribozymes, RNA, and DNA.

The term “polypeptide” refers to a polymer composed of multiple amino acids. A protein can be an example of a polypeptide.

The term “enzyme” refers to a macromolecule, usually a protein, that functions as a (bio) catalyst by increasing the reaction rate of a chemical or biochemical reaction. In general, an enzyme catalyzes only one reaction type (i.e., reaction selectivity) and operates on only one type of substrate (i.e., substrate selectivity). Substrate molecules are transformed at the same site (regioselectivity) and generally, only one chiral substrate of a racemic substrate pair is transformed (enantioselectivity, a special form of stereoselectivity).

The term “nucleic acid” refers to a polymer composed of nucleotide subunits. The nucleotide subunits can be joined together through phosphodiester bonds.

The term “receptor-ligand binding pair” refers to a complex having a receptor and a ligand that are generally held together in a reversible manner, by noncovalent interactions (e.g., hydrogen bonding, ionic interactions, or hydrophobic interactions).

The term “titrant” refers to the substance (e.g., a ligand) that quantitatively reacts and/or interacts with the analyte (e.g., a receptor) in a titration. The titrant is usually a standard solution added carefully to the analyte until the reaction and/or interaction is complete. The amount of analyte is calculated from the volume of titrant required for complete reaction.

The term “titration” refers to a procedure in which one solution (e.g., a solution of ligand having a known K_{d}) is added to another solution (e.g., a mixture of substrate and a ligand wherein the K_{d} is unknown) until the interaction between the two solutes (e.g., the substrate and the ligand having an estimated K_{d}) is complete, wherein the concentration of one solution of ligand is known, or known approximately (e.g., the concentration of the ligand having the known or estimated K_{d}).

The term “equilibrium” refers to a state in a reversible chemical and/or biochemical reaction and/or interaction at which the reactants are turning into products at the same rate as the products are turning back into the reactants, so that the amounts of each reactant and product remains essentially constant.

The phrase “disrupting the receptor-ligand binding pairs” refers to causing the receptor-ligand binding pair to dissociate into unbound receptor and ligand. Disruption of the receptor-ligand binding pairs can be accomplished by a variety of methods such as use of chromatography (e.g., high resolution reverse phase chromatography under high temperature); change of pressure, pH, salt concentration, temperature or organic solvent concentration; or competition with a known ligand to the receptor, or any combination of these techniques.

The term “liquid chromatography” refers to an analytical chromatographic technique that is used to separate ions and/or molecules that are dissolved in a solvent. If the sample solution is in contact with a second solid or liquid phase, the different solutes will interact with the other phase to differing degrees due to differences in adsorption, ion-exchange, partitioning, or size. These differences allow the mixture components to be separated from each other by using these differences to determine the transit time of the solutes through a column. High-performance liquid chromatography (HPLC) is a form of liquid chromatography to separate compounds that are dissolved in solution. HPLC instruments consist of a reservoir of mobile phase, a pump, an injector, a separation column, and a detector. Compounds are separated by injecting a plug of the sample mixture onto the column. The different components in the mixture pass through the column at different rates due to differences in their partitioning behavior between the mobile liquid phase and the stationary phase.

The term “competitive binder” refers to a ligand that binds to a receptor at a specific site (e.g., a catalytic site of an enzyme), where it competes with another ligand for binding in a dynamic equilibrium-like process.

The term “mass-encoded” refers to a quality of a set of chemical compounds (e.g., related compounds, initial compounds, variants or any combination thereof), where at least about 90% of the individual compounds in the set of compounds have a molecular mass that is distinct from the molecular masses of all of the other chemical compounds in the set.

The term “threshold binding characteristic” refers to any defined property of a ligand that provides an objective identification of the ability of that ligand to associate with a target. For example, a threshold-binding characteristic can be defined as a particular K_{d} of a ligand, for example <50 μM, <20 μM, <10 μM, <1 μM, <0.5 μM, <0.1 μM, <0.01 μM, etc. The threshold binding characteristic can be defined qualitatively or quantitatively relative to another known ligand, e.g., standard compound, as selected or desired by the user. Alternatively the threshold binding characteristic of a ligand can be defined qualitatively relative to other ligands, for example, a threshold binding characteristic can be defined as the top binding ligands within a mixture, for example, a threshold binding characteristic can be ligands which bind more tightly than ½ the ligands in the mixture, ligands that bind more tightly than ¾ of the ligands in the mixture, or ligands that bind more tightly than a known ligand in the mixture. The same types of quantitative and relative binding characteristics can also be used by analysis of the k_{off}, the half-life, t_{1/2}, or other binding characteristics of a ligand.

As can be appreciated by the skilled artisan, methods of synthesizing compounds (e.g., structurally related variants) for use in the present invention will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Additionally, compounds and/or libraries of compounds can be purchased from commercial sources. Furthermore, the compounds can by made by a combination of purchase of precursor compounds, followed by additional synthetic manipulations, thus providing a desired compound.

### EXAMPLES

### Example 1

### Determination of K_{d} for Human Serum Albumin (HSA) and the small molecule Warfarin

Six experimental samples containing 0.125 pmol, 0.25 pmol, 0.5 pmol, 1 pmol, 2, pmol, and 4 pmol of warfarin are analyzed to establish a calibration curve relating the mass spectrometric signal for warfarin. To improve the accuracy of the calibration, two data points are obtained for the 0.125 pmol sample.

Seven experimental samples containing known concentrations of warfarin, [S]₀, are prepared, by serial dilution. In particular, calibrated signal intensities for warfarin are measured in duplicate on 2 uL samples consisting of 80, 40, 20, 10, 5, 2.5 or 1.0 uM total [warfarin]. To each of these samples is added a constant quantity of HSA whose concentration is known nominally but not precisely. In this case, 0.2 uM nominal [HSA] was used. The sample solutions are incubated together for a time sufficient to allow the receptor-ligand (HSA-warfarin) complex to reach equilibrium, roughly 30 minutes.

The solution of HSA, unbound warfarin, and HSA-warfarin complex is then passed through a size exclusion chromatography stage to separate the HSA plus HSA-warfarin complex from unbound warfarin on the basis of molecular size, with the HSA plus HSA-warfarin complex co-eluting at the front of the eluant stream.

The portion of the eluant stream containing the HSA plus HSA-warfarin complex but not containing any unbound warfarin is diverted to a reverse-phase chromatography stage for desalting and elution into a mass spectrometer. The reverse-phase chromatography essentially disrupts the HSA-warfarin complex. After separation, the warfarin is identified on the basis of its molecular weight or mass spectrometry-mass spectrometry fragmentation pattern. The warfarin is then quantified by measurement of its calibrated signal response.

Because any amount of warfarin that is not bound to the HSA is separated from the HSA-warfarin complex by size exclusion, any warfarin reaching the mass spectrometer (i.e., recovered ligand) comes from the HSA-warfarin complex. Therefore, the calibrated signal for the warfarin is correlated to the amount of HSA-warfarin complex present at the beginning of the size exclusion chromatography. [HSA-Warfarin] and [Warfarin] data pairs are fit to the formula below:

$${K}_{d} = \frac{( {[ E ]}_{0} - [ ES ] ) ( {[ S ]}_{0} - [ ES ] )}{[ ES ]}$$

to yield best-fit values for the parameters K_{d} and [HSA]₀, by numerical non-linear regression techniques.

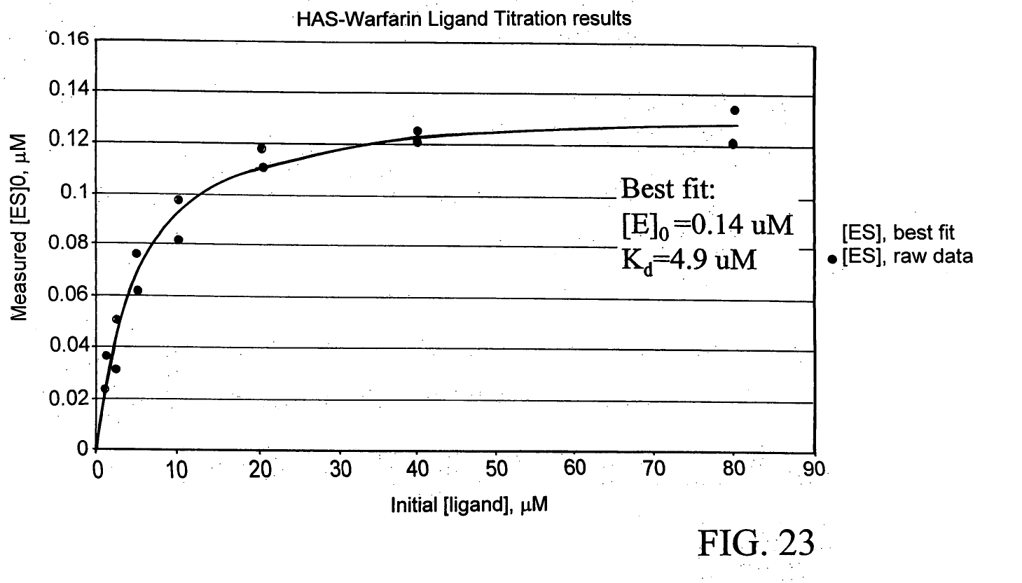

The results of the above experiment are shown in FIG. 23. The parameters K_{d} and [HSA]₀ were estimated by numerical non-linear regression techniques to be 4.9 uM and 0.14 uM, respectively. The value for the K_{d} is in good agreement with literature values; the value for [HSA]₀ indicates that ˜75% of the nominal protein concentration is active, i.e., competent to bind ligand in an experiment.

### Example 2

### Receptor-Ligand System of Porcine Muscarinic Receptor (pM2R)

A series of seven experimental samples containing a constant concentration (4.0 μM) of Porcine Muscarinic Receptor (pM2R), a constant concentration, 1 μM, of a mixture of ligands Sᵢ (See Table 1 below), and increasing concentrations (0, 0.1, 0.22, 0.46, 1.0, 2.2, 4.6, 10, 22, 46, and 100 μM) of another, known ligand [S₁]₀ (Atropine) are prepared. The samples are incubated for a time sufficient to allow the receptor-ligand complex forming reactions in each of the samples to reach equilibrium, roughly 30 minutes.

The solutions of pM2R, unbound ligands, and pM2R-ligand complexes are then passed through size exclusion chromatography to separate the pM2R plus pM2R-ligand complex from unbound ligands on the basis of molecular size, with the pM2R plus pM2R-ligand complex co-eluting at the front of the eluant stream. The portion of the eluant stream containing the pM2R plus pM2R-ligand complex but not containing any unbound ligand is then diverted to a reverse-phase chromatography stage for desalting, followed by elution into a mass spectrometer. The reverse-phase chromatography disrupts and separates the receptor-ligand binding pair. Thus, after reverse-phase chromatography, the ligand is subjected to Mass Spectroscopy (MS) and identified on the basis of its molecular weight or mass spectrometry-mass spectrometry fragmentation pattern.

Any amount of ligand that is not bound to the pM2R is separated from the pM2R-ligand complex by the size exclusion step. Therefore, any ligand reaching the mass spectrometer (i.e., recovered ligand) comes from the corresponding pM2R-ligand complex, and the mass spectrometric signal for each ligand Sᵢ correlates to the amount of pM2R-ligand complex, RLᵢ, present at the beginning of the size exclusion chromatography.

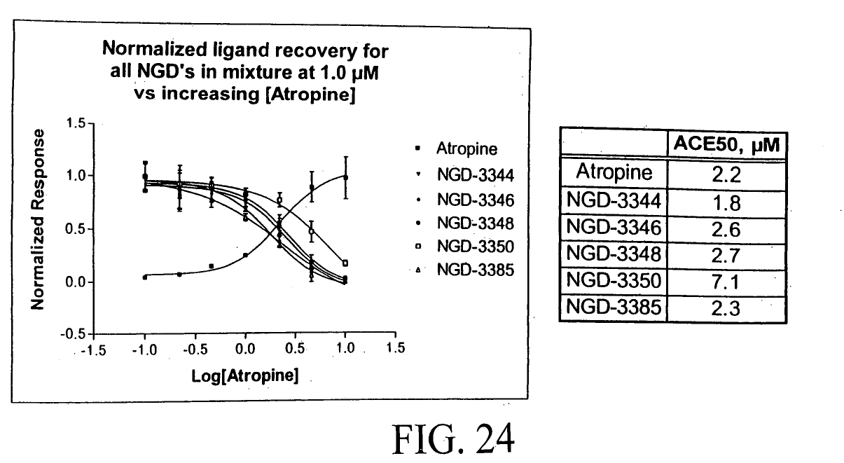

Ligand recoveries for each of the experimental samples having constant concentrations of the ligands and pM2R and increasing concentration of the, added competitor Atropine are plotted and the 50% inhibitory concentration point (ACE₅₀ value) is determined for each ligand in the mixtures (See FIG. 24). Qualitative compound affinity is determined according to the ACE₅₀ value. The higher the ACE₅₀ value, the lower the K_{d}.

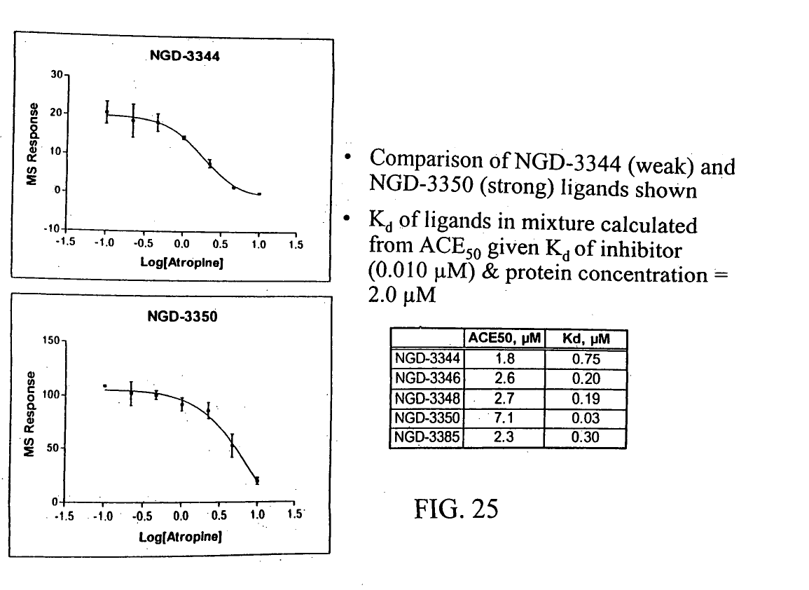

For quantitative K_{d} determination, the ACE₅₀ values, the initial pM2R receptor concentration [E]₀, initial ligand concentration of the compounds in the mixture under study [Sᵢ], and competitor Atropine K_{d} are entered into the equation shown in FIG. 6, which is a solution to the equation of formula (I). The results are shown in FIG. 25.

TABLE 1
Table of NGD structures

| Name | Structure |
|---|---|
| NGD-3344 | 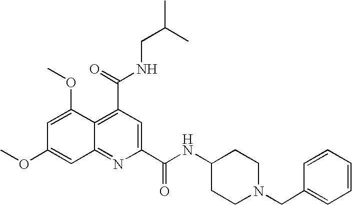 |
| NGD-3346 | 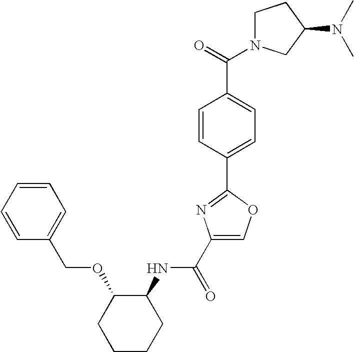 |
| NGD-3348 | 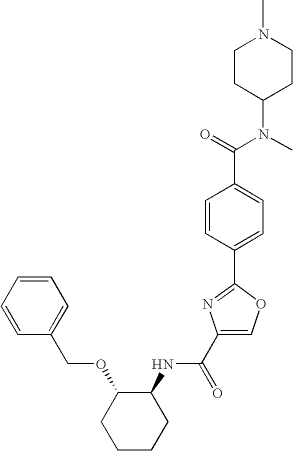 |
| NGD-3350 | 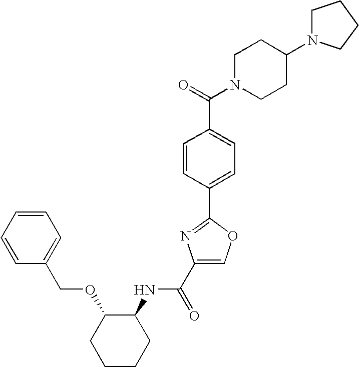 |
| NGD-3385 | 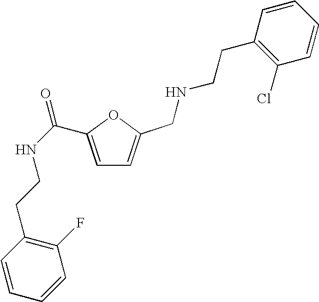 |

### Example 4

### Receptor-Ligand System of Serine Kinase Zap-70, NGD-6380, and Staurosporine

A mixture of 9 μM Zap-70 and 1 μM NGD-6380 is prepared and incubated at room temperature for 30 minutes, a time sufficient for the mixture to reach equilibrium of bound and unbound components. To the mixture of Zap-70 and NGD-6380 is added a mixture containing 100 μM of Staurosporine and 1 μM NGD-6380 (keeping the concentration of ligand unchanged with the addition of inhibitor). Table 2 depicts structures of Staurosporine and NGD-6380.

The resulting dissociation of NGD-6380 is measured over time at intervals of 20, 30, 40, 60, 70, 80, and 100 minutes. The dissociation of NGD-6380 is measured in the following manner. The aliquots of the solution of Zap-70, unbound NGD-6380, and Zap-70-NGD-6380 complexes are passed through size exclusion chromatography to separate the Zap-70 plus Zap-70-NGD-6380 complexes from unbound NGD-6380 on the basis of molecular size, with the Zap-70 plus Zap-70-NGD-6380 complex co-eluting at the front of the eluant stream. The portion of the eluant stream containing the Zap-70 plus Zap-70-NGD-6380 complex but not containing any unbound NGD-6380 is then diverted to a reverse-phase chromatography stage for desalting and separation of the receptor-ligand binding pair. Thus, after reverse-phase chromatography, the NGD-6380 is subjected to Mass Spectroscopy (MS) wherein the signal response is recorded.

Any amount of NGD-6380 which is not bound to the Zap-70 is separated from the Zap-70-NGD-6380 complex by the size exclusion step. Therefore, any NGD-6380 reaching the mass spectrometer (i.e., recovered ligand) comes from the corresponding Zap-70-NGD-6380 complex, and the mass spectrometric signal for NGD-6380 correlates to the amount of Zap-70-NGD-6380 complex present at the beginning of the size exclusion chromatography. Thus, the dissociation of Zap-70 from the Zap-70-NGD-6380 complex is seen by the decreasing amount of the signal response of the NGD-6380 over time.

The decrease in the signal response of the NGD-6380 is plotted as a function of time and the experimental data is fit to the exponential decay function of formula (XVIII) using the program Mathematical (Wolfram Research Inc., version 4.1).
[ES]=[ES]₀e^{-ks2·t} formula (XVIII)

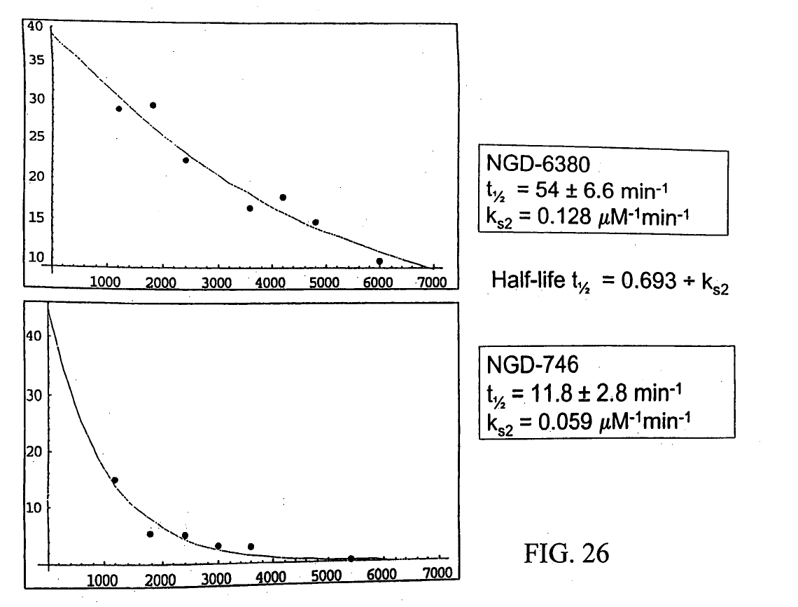

The results showing both the dissociation rate kₛ₂ and the half-life t_{1/2} are in FIG. 26.

TABLE 2
Table of structures of Staurosporin and NGD-6380

| Name | Structure |
|---|---|
| Staurosporine | 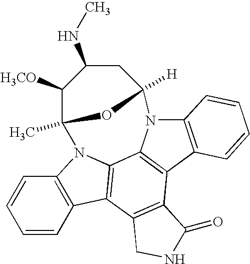 |
| NGD-6380 | 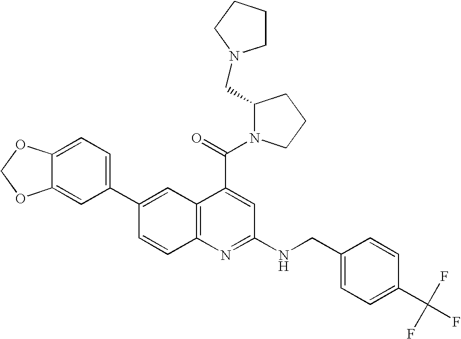 |

### Example 4

### Receptor-Ligand System of Serine Kinase Zap-70, NGD-746, and Staurosporine

A mixture of 9 μM Zap-70 and 1 μM NGD-746 is prepared and incubated at room temperature for 30 minutes, a time sufficient for the mixture to reach equilibrium of bound and unbound components. To the mixture of Zap-70 and NGD-746 is added a mixture containing 100 μM of Staurosporine and 1 μM NGD-746 (keeping the concentration of ligand unchanged with the addition of inhibitor). Table 3 depicts structures of Staurosporine and NGD-746.

The resulting dissociation of NGD-746 is measured as in Example 3 over time at intervals of 20, 30, 40, 50, 60, and 90 minutes. The decrease in the signal response of the NGD-746 is plotted as a function of time and the experimental data is fit to the exponential decay function of formula 8 using the program Mathematica®.
[ES]=[ES]₀e^{-ks2·t} formula (XVII)

The results showing both the dissociation rate kₛ₂ and the half-life t_{1/2} are in FIG. 26.

TABLE 3
Table of Staurosporine and NGD-746

| Name | Structure |
|---|---|
| Staurosporine | 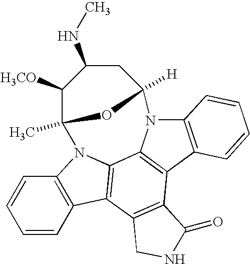 |
| NGD-746 | 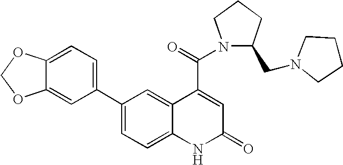 |

### Example 5

### Receptor-Ligand System of Serine Kinase Zap-70, a Mixture of Ligands, and Staurosporine

A mixture of 9 μM Zap-70 and 1 μM of a mixture of the ligands depicted in Table 3 is prepared and incubated at room temperature for 30 minutes, a time sufficient for the mixture to reach equilibrium of bound and unbound components. To the mixture of Zap-70 and the ligands is added a mixture containing 100 μM of Staurosporine and 1 μM of the ligands (keeping the concentration of ligands unchanged with the addition of inhibitor).

The resulting dissociation of ligands was measured as in Example 3 over time at intervals of 20, 30, 40, 50, 60, 70, 80, 90, 100, 10, and 120 minutes. The decrease in the signal response of the ligands is plotted as a function of time and the experimental data is fit to the exponential decay function of formula (XVIII) using the program Mathematica®.
[ESᵢ]=[ESᵢ]₀e^{-ks2·t} formula (XVIII)

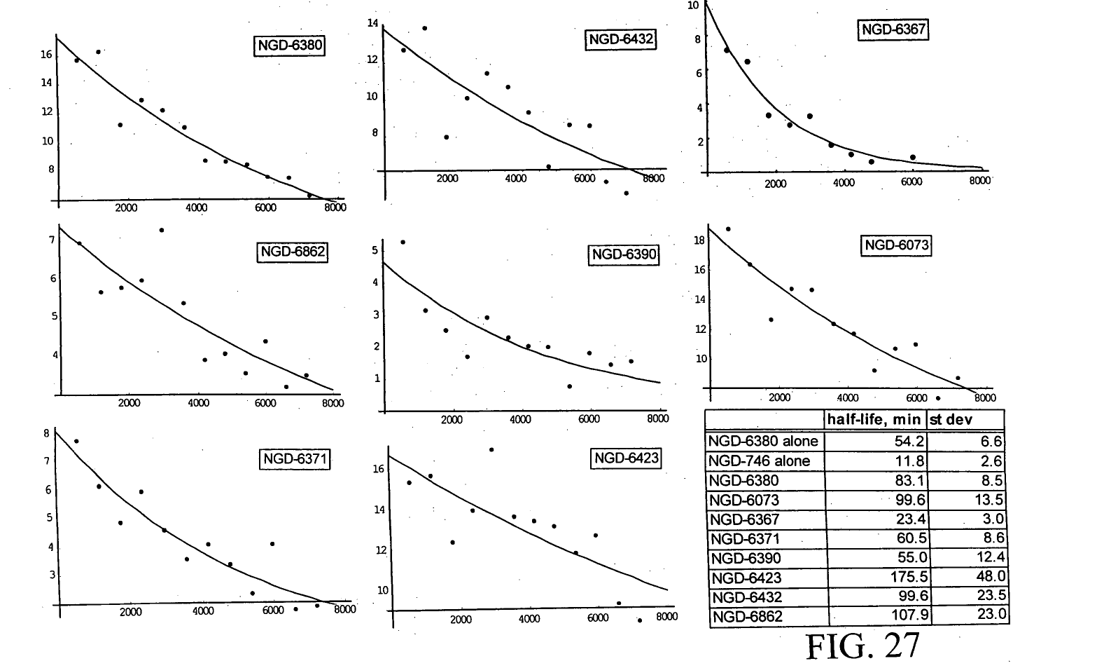

The results showing the half-life t_{1/2} are in FIG. 27. The dissociation rate kₛ₂ are shown in Table 4.

TABLE 4
Table of Staurosporine and NGD structures

| | | Dissociation rate kₛ₂ |
|---|---|---|
| Name | Structure | (μM⁻¹ min⁻¹) |
| Staurosporine | 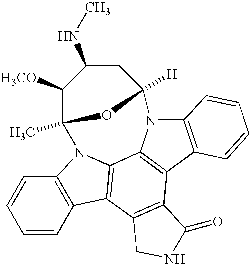 | — |
| NGD-6380 | 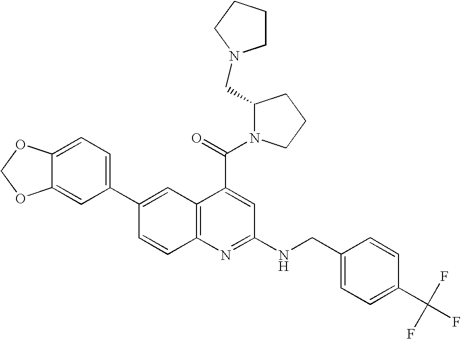 | 0.0083 |
| NGD-6073 | 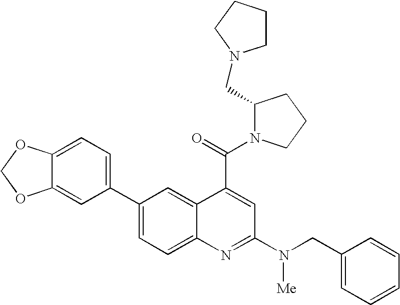 | 0.0070 |
| NGD-6367 | 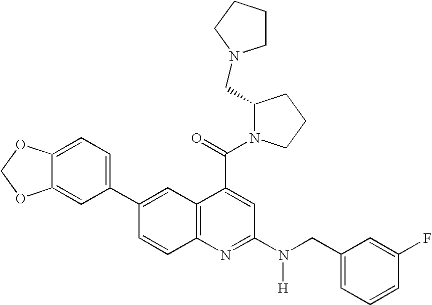 | 0.0296 |
| NGD-6371 | 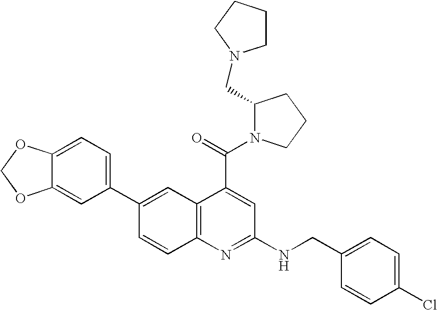 | 0.0165 |
| NGD-6390 | 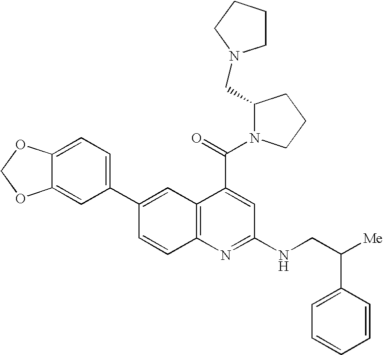 | 0.0126 |
| NGD-6423 | 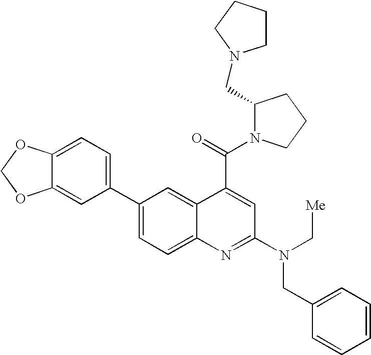 | 0.0039 |
| NGD-6432 | 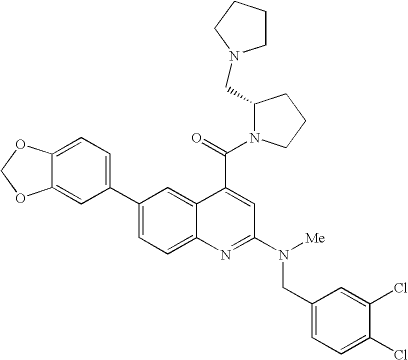 | 0.0070 |
| NGD-6862 | 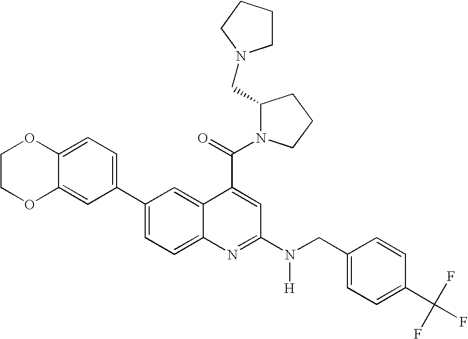 | 0.0064 |

### Example 6

### Mixture-Based Affinity Selection-Mass Spectrometry K_{d} Evaluation by Competition Between Increasing Concentrations of a Competitor Ligand and the Originator Ligand plus Structural Variants

The following steps are executed for the protein-ligand model system of ZAP-70 Kinase, the known, initial ligand NGD-746 discovered via high-throughput screening techniques, and mass-encoded mixture of structural variants of NGD-746.

Experimental samples containing a 10 μM nominal concentration of receptor ZAP-70, a constant concentration (1 μM) of a mixture of ligands, including originator ligand NGD-746, and increasing concentrations (0, 0.22, 0.46, 1, 2.2, 4.6, 10, 22, 46, 100 μM) of the Zap-70 ligand Staurosporine are prepared. The samples are incubated for a time sufficient to allow the receptor-ligand complex forming reactions in each of the samples to reach equilibrium, roughly 30 minutes.

The solutions of ZAP-70, unbound ligands, and ZAP-70-ligand complexes are then passed through size exclusion chromatography to separate the ZAP-70 plus ZAP-70-ligand complexes from unbound ligands on the basis of molecular size, with the ZAP-70 plus ZAP-70-ligand complexes co-eluting at the front of the eluant stream. The portion of the eluant stream containing the ZAP-70 plus ZAP-70-ligand complexes but not containing any unbound ligand is then diverted to a reverse-phase chromatography stage for desalting, followed by elution into a mass spectrometer. The reverse-phase chromatography disrupts and separates the receptor-ligand binding pairs. Thus, after reverse-phase chromatography, the ligands are subjected to Mass Spectroscopy (MS) wherein the signal response is determined and the ligands are identified on the basis of molecular weight or mass spectrometry-mass spectrometry fragmentation patterns.

Any amount of ligand that is not bound to ZAP-70 is separated from the ZAP-70-ligand complexes by the size exclusion step. Therefore, any ligands reaching the mass spectrometer (i.e., recovered ligands) come from the corresponding ZAP-70-ligand complexes, and the mass spectrometric signal for each ligand correlates to the amount of ZAP-70-ligand complex present at the beginning of the size exclusion chromatography.

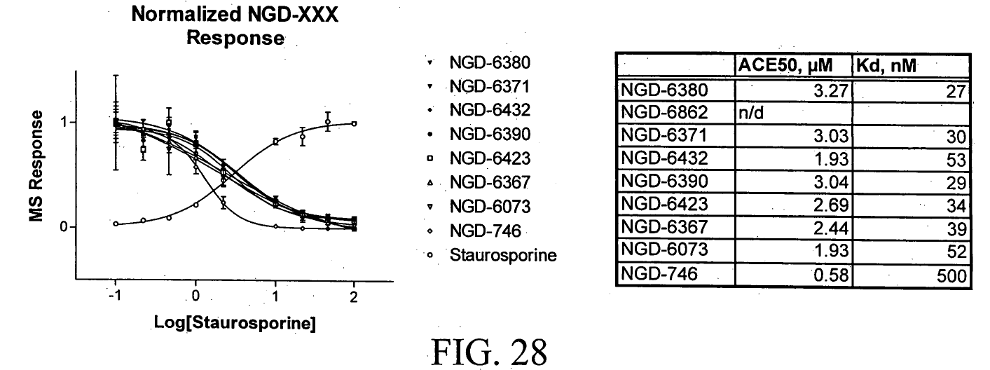

Normalized ligand recoveries for each mixture component and the competitor ligand in each of the experimental samples are shown in FIG. 28. The concentration of the competitive ligand Staurosporine required to reduce the response of each ligand to one-half its value in the absence of competitor, the ACE₅₀ value for each ligand, correlates inversely with the K_{d} of the ligand. The ACE₅₀ values, and the K_{d} values calculated from each ACE₅₀ value, for each structural variant in the mixture are also shown in FIG. 28. These data are used to identify ligands having a binding characteristic of interest and to generate a subsequent series of optimization mixtures (e.g., a new mixture of variant compounds).

### Example 7

### Synthesis of Initial Ligand NGD-746 and a Mass-Encoded Mixture of its Derivatives: NGD-6037, 6367, 6371, 6380, 6390, 6423, 6432, 6862

5-Iodoisatin 1 (10 g, 36.3 mmol) and malonic acid (7.5 g, 72 mmol) in 200 mL of glacial acetic acid were refluxed overnight. The precipitate was collected by filtration, washed with AcOH and acetone. The solid was then refluxed with EtOH for 1 h. Filtration and washing with EtOH and Et₂O gave 6-Iodo-2-oxo-1,2-dihydro-quinoline-4-carboxylic acid 2 as the product, yielding 8.8 g (76%). ¹H-NMR (400 MHz, DMSO-d₆): δ 14.0 (br s, 1H), 12.13 (s, 1H), 8.56 (d, 1H, J=8.1 Hz), 7.83 (dd, 1H, J=8.7, 1.8 Hz), 7.17 (d, 1H, J=8.4 Hz), 6.93 (s, 1H).

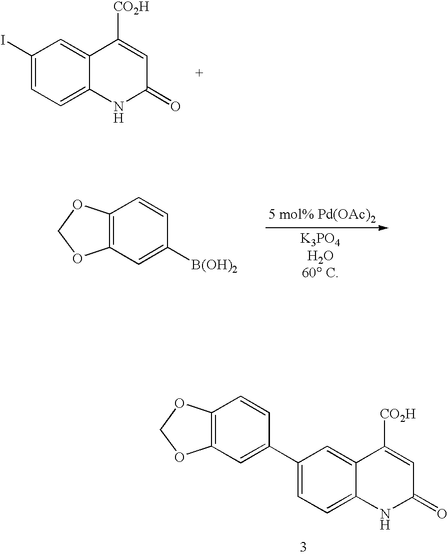

A mixture of 6-Iodo-2-oxo-1,2-dihydro-quinoline-4-carboxylic acid 2 (3.15 g, 10 mmol), 3,4-(methylenedioxyl)phenylboronic acid (2.49 g, 15 mmol), K₃PO₄ (8.49 g, 40 mmol), and Pd(OAc)₂ (112 mg, 0.5 mmol) in 60 mL of degassed H₂O was heated under argone at 60° C. for 2 h. After cooling to room temperature, the solid was collected by filtration, washed with H₂O and acetone. It was then treated with 20 mL of 1M HCl, the resulting greenish yellow solid was filtered again and washed with H₂O. Drying in vacuo over P₂O₅ yielded 2.53 g (82%) of product 3 as a greenish yellow solid. ¹H-NMR (400 MHz, DMSO-d₆): δ 14.0 (br s, 1H), 12.11 (s, 1H), 8.34 (s, 1H), 7.79 (d, 1H, J=7.6 Hz), 7.40 (d, 1H, J=8.6 Hz), 7.18 (s, 1H), 7.08 (d, 1H, J=7.1 Hz), 7.01 (d, 1H, J=8.2 Hz), 6.91 (s, 1H), 6.06 (s, 2H).

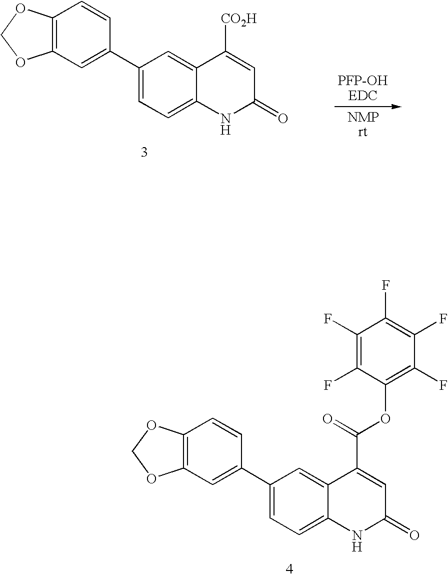

A mixture of acid 3 (2.0 g, 10.57 mmol), pentafluorophenol (2.92 g, 15.85 mmol), and EDC (3.04 g, 15.85 mmol) in 30 mL of NMP was stirred at room temperature for 6 h. Filtered the precipitate with the aid of NMP, the solution was poured into 60 mL of ice water. The resulting solid was collected by filtration, washed with ice water, and EtOAc, and dried in vacuo to yield ester 4 (2.80 g, 91%) which was used for the next step without further purification.

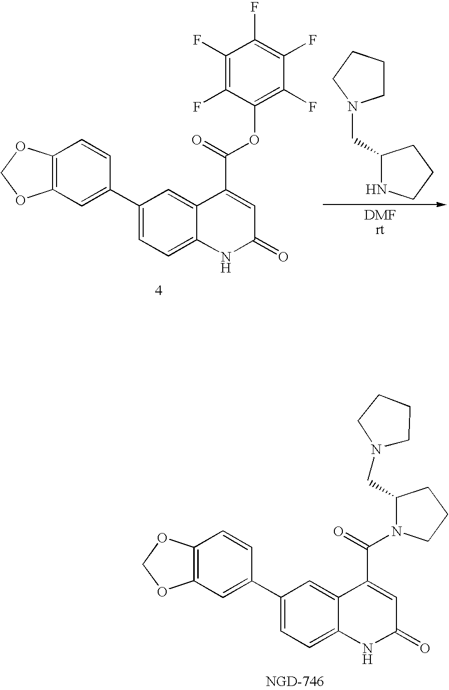

Ester 4 (12 mg, 0.025 mmol) in 0.5 mL of DMF was added into a mixture of pyrolidinopyrrolidine (7.7 mg, 0.05 mmol) and Amberlite® basic resin (50 mg) in 1 mL of DMF. The reaction mixture was allowed to stir at room temperature for 12 h, isocyanate resin (90 mg) was added, and continued to stir for 3 h. The solution then was collected by filtration and concentrated in vacuo to yield product NGD-746. MS m/z 446.2 (M⁺+1).

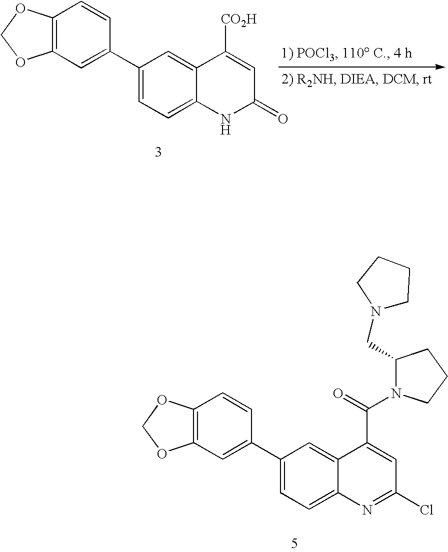

6-Benzo[1,3]dioxol-5-yl-2-oxo-1,2-dihydro-quinoline-4-carboxylic acid 3 (1.0 g, 3.2 mol) in 10 mL of oxyphosphourus chloride was refluxed for 4 h and cooled to room temperature. The solution was concentrated to dryness to yield a brownish yellow solid. The solid was then dissolved in 20 mL of methylene dichloride. Diisopropylethylamine (1.50 g, 11.5 mmol) and 2-(S)-pyrroldinylmethylpyrrolidine (0.59 g, 3.84 mmol) were slowly added into the solution at 0° C. The mixture was allowed to stir at room temperature for 12 h. After removing the solvent by rotary evaporation, the residue was dissolved in ethyl acetate, washed with saturated aqueous NaHCO₃ and brine. The organic phase was dried over sodium sulfate and concentrated. The residue was purified by silica gel column chromatography (Et₃N—AcOEt 5:95) to give (6-Benzo[1,3]dioxol-5-yl-2-chloro-quinolin-4-yl)-(2-pyrrolidin-1-ylmethyl-pyrrolidin-1-yl)-methanone 5 (1.20 g, 81%). MS m/z 464.2 (M⁺+1); ¹H-NMR (400 MHz, CDCl₃): δ 8.05 (m, 1H), 7.93 (m, 1H), 7.85 (br s, 1H), 7.41 (s, 1H), 7.13 (m, 1H), 7.11 (s, 1H), 6.92 (m, 1H), 6.03(s, 1H), 4.58 (m, 1H), 3.92 (m, 0.5H), 3.75 (m, 0.5H), 3.38-3.13 (m, 2H), 2.94 (m, 3H), 2.35-2.15 (m, 2H), 2.04 (m, 3H), 2.01-1.85 (m, 4H), 1.83 (m, 1H).

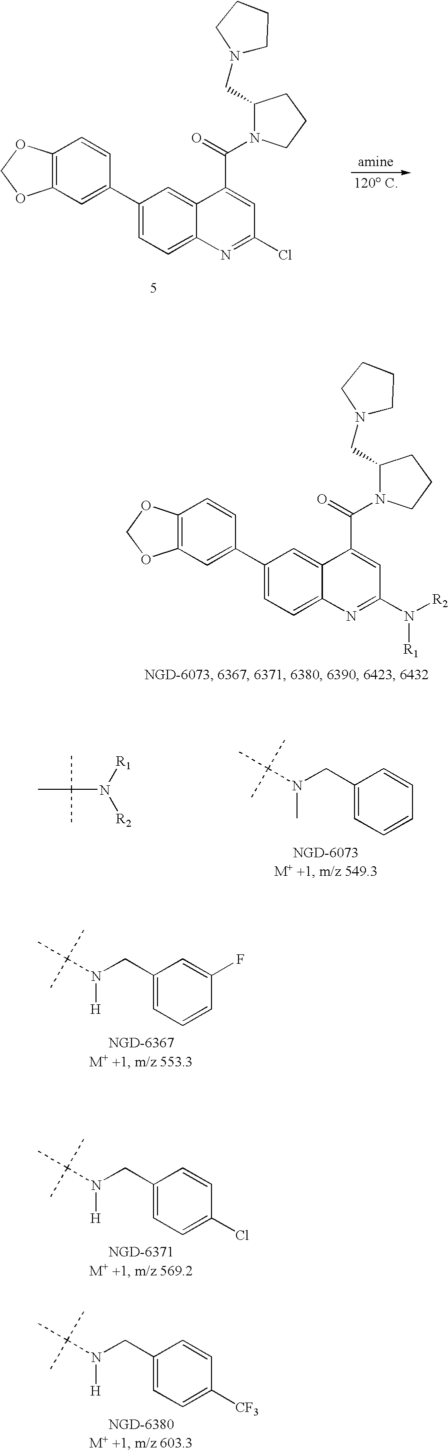

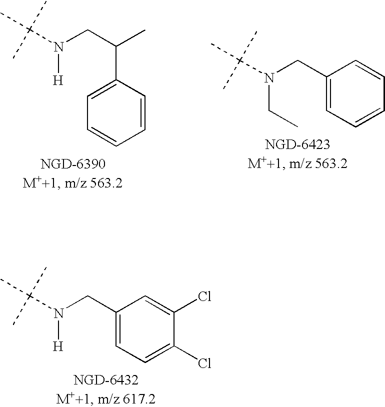

2-Chloroquinoline 5 (20 mg, 0.0425 mg) and N-methylbenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH_{3 CN (}3:1), and purified by preparative LC to yield NGD-6073. MS m/z 549.3 (M⁺+1).

2-Chloroquinoline 5 (20 mg, 0.0425 mg) and 3′-fluorobenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6367. MS m/z 553.3 (M⁺+1).

2-Chloroquinoline 5 (20 mg, 0.0425 mg) and 4′-chlorobenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6371. MS m/z 569.2 (M⁺+1).

2-Chloroquinoline 5 (20 mg, 0.0425 mg) and 4′-trifluoromethylbenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6380. MS m/z 603.3 (M⁺+1).

2-Chloroquinoline 5 (20 mg, 0.0425 mg) and 2-methylphenethylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6390. MS m/z 563.2 (M⁺+1).

2-Chloroquinoline 5 (20 mg, 0.0425 mg) and N-ethylbenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6423. MS m/z 563.2 (M⁺+1).

2-Chloroquinoline 5 (20 mg, 0.0425 mg) and N-methyl-3′,4′-dichlorobenzylamine (0.1275 mmol, 3 equiv) in 0.3 mL of NMP was heated for 12 h at 120° C. LC-MS analysis indicated that the reaction was complete. The reaction mixture was then dissolved in 1 mL of DMSO/CH₃CN (3:1), and purified by preparative LC to yield NGD-6432. MS m/z 617.2 (M⁺+1).

6-Iodo-2-oxo-1,2-dihydro-quinoline-4-carboxylic acid 2 (5.97 g, 18.93 mmol) in 20 mL of oxyphosphourus chloride was heated upto 100° C. for 4 h and then cooled to room temperature. The solution was concentrated to dryness to yield a brownish yellow solid. The solid was then dissolved in 100 mL of methylene dichloride. Diisopropylethylamine (20 ml, about 100 mmol) and 2-(S)-pyrroldinylmethylpyrrolidine (3.5 g, 22.7 mmol) were slowly added into the solution at 0° C. The mixture was allowed to stir at room temperature overnight. The mixture was diluted by methylene dichloride (200 ml), washed with water (2×40 ml), saturated aqueous

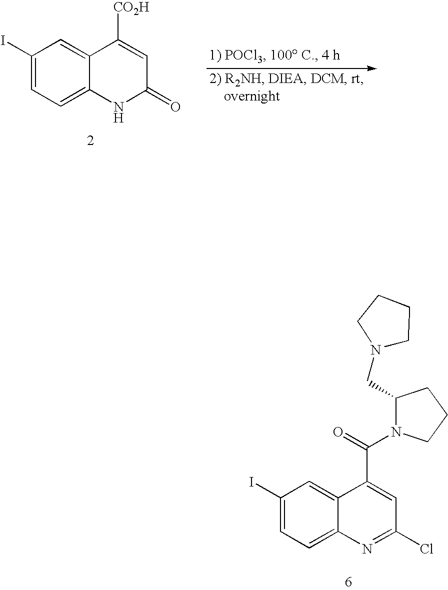

NaHCO₃ and brine. The organic phase was dried over Na₂SO₄ and concentrated to give the crude product 6 (6.5 g). With LC-MS, this compound was pure enough for the next step and was used directly without any purification. MS m/z 470.0 (M⁺+1).

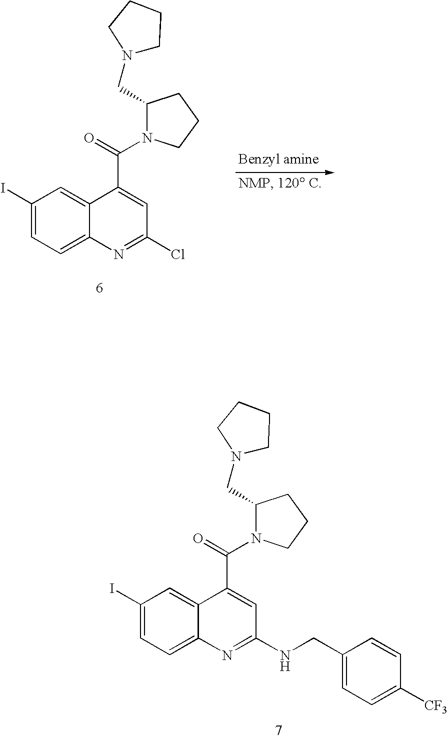

2-Chloroquinoline compound 6 (3.12 g, 6.64 mg) and p-trifluorobenzylamine (5.81 g, 33 mmol) in NMP (20 ml) was heated at 120° C. for 36 h. LC-MS analysis indicated that the reaction was complete. The reaction mixture was then cooled to room temperature and diluted by ethyl acetate (200 ml). The organic phase was washed by water and brine and dried over Na₂SO₄. Concentration gave the crude product and the residue was purified by silica gel column chromatography (Et₃N—CH₂Cl₂—MeOH 2:95:5) to give the desired product 7 (3.2 g). MS m/z 609.1 (M⁺+1).

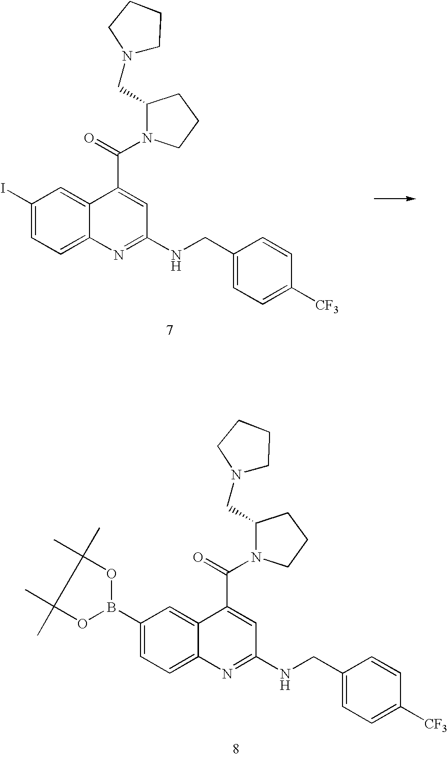

To a 25 ml round bottom flask charged with bis(pinacolato)diboron (279 mg, 1.1 mmol), KOAc (294, 3.0 mmol) and PdCl₂(dppf) (24.5 mg, 0.03 mmol) was added a solution of 6-iodoquinoline 7 (607 mg, 1.0 mmol) in DMSO (6 mL). The mixture was thoroughly degassed by alternately connected the flask to vacuum and argon. This resulting mixture was then heated at 80° C. overnight, diluted by EtOAc (40 mL) and filtered through Celite. The resulting product 8 was used in next steps without further purification after concentration. MS m/z 609 (M⁺+1).

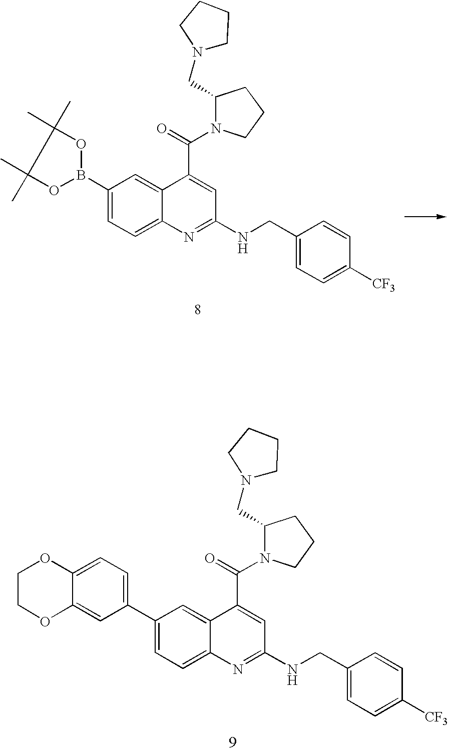

Under Ar, the 6-boronate 8 (15 mg, 0.025 mmol) in dioxane (2.0 mL) was added to the flask which was charged with Pd(dppf)Cl₂ (2 mg), Cs₂CO₃ (17 mg, 0.055 mmol), and 3,4-ethylenedioxyiodobenzene (15 mg, 0.057 mmol). The mixture was thoroughly degassed by alternately connected the flask to vacuum and argon. The resulting solution was heated to 70° C. and stirred overnight. It was diluted with EtOAc after cooling to room temperature. The solid was removed by filter through Celite and washed by some EtOAc. The organic phase was concentrated to remove the solvent. The resulting residue was purified by preparative LC to give product 9 (NGD-6862). MS m/z 617 (M⁺+1).

The mass-encoded mixtures of the above NGD compounds were simply made by mixing NGD-6037, 6367, 6371, 6380, 6390, 6423, 6432 and NGD-6862 in various concentrations as needed for the following experiments. The same mixtures can also be made by one of skill in the art directly from mixture-based synthesis or alternate methods known in the art.

### Example 8

### Sample Transfer from SEC to MS Through Sample Loop

Human Serum Albumin is combined with a plurality of ligands, and the resulting mixture is incubated in a buffered solvent for a time sufficient to allow the receptor-ligand complexes to reach equilibrium. Once the mixture has reached equilibrium, the mixture is injected on a size-exclusion column. As the receptor-ligand complexes are eluted from the size-exclusion column, they are detected by a UV detector. Upon detection, a controller run by a computer begins a timer that is calibrated for a time sufficient to fill the a sample loop connected to a 2-position 6-port selection valve with a representative sample of the receptor-ligand mixture. Once the sample loop is filled with receptor ligand complex, the controller shifts the position of the sample loop in the selection valve to load the receptor ligand complex onto a reverse phase liquid chromatography (RP-LC) device. While on the RP-LC, the receptor-ligand complexes are disrupted, dissociating the receptors from the ligands. Upon elution from the RP-LC, the dissociated ligands are then transferred to a mass-spectrometer where they are identified.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Accordingly, other embodiments are within the scope of the following claims.

1. A method of analyzing a binding affinity of a receptor for a ligand in a plurality of mixtures comprising a receptor, E, a ligand, Sᵢ, and a receptor-ligand binding pair, ESᵢ, the method comprising;
(a) providing a plurality of mixtures, each mixture comprising a receptor, [E]₀, a ligand [Sᵢ]₀, and a titrant, T, wherein the concentration of one or more of E, Sᵢ, T are chosen such that the relative ability of T to displace S can be determined;
(b) allowing each of the plurality of mixtures to achieve equilibrium;
(c) separating the receptor-ligand binding pairs, ESᵢ, from the unbound ligands, Sᵢ, for each of the plurality of mixtures;
(d) determining the signal response from an analytical device for the receptor-ligand binding pair, ESᵢ in each of the plurality of mixtures; and
(e) evaluating the signal responses from step (d) of the receptor-ligand binding pairs, ESᵢ, to determine binding affinity of the ligand Sᵢ to the receptor E.

2. The method of claim 1, wherein each mixture is selected such that the concentration of T relative to [E]₀ and [Sᵢ]₀, is chosen to allow for comparison of the relative ability of Sᵢ to displace T.

3. The method of claim 1, comprising providing a plurality of mixtures, each mixture comprising a initial concentration of receptor, [E]₀, an initial concentration of ligand [Sᵢ]₀, and a known concentration of a titrant wherein [E]₀ and [Sᵢ]₀ are constant throughout each of the plurality of mixtures, the [Sᵢ]₀ is approximately the same within each of the plurality of mixtures, and the concentration of the titrant is varied within the plurality of mixtures.

4. The method of claim 1, wherein the plurality of mixture each comprise a plurality of ligands Sᵢ, and a plurality of receptor-ligand binding pairs, ESᵢ, and wherein the signal response is determined for at least two of the receptor-ligand binding pairs, ESᵢ, and the relative binding of the receptor-ligand binding pairs, ESᵢ, is determined.

5. The method of claim 4, wherein at least about 90% of the plurality of ligands, Sᵢ, have a unique molecular mass.

6. The method of claim 1, wherein each mixture is selected such that the concentration of T relative to [E]₀ and [Sᵢ]₀, is chosen such that the binding affinity of a first ligand, S₁, can be compared with the binding affinity of a second ligand, S₂, to provide a measure of the relative binding affinity of S₁ for E and S₂ for E.

7. The method of claim 1, wherein the binding affinities are relative binding equilibrium constants, K_{di}s.

8. The method of claim 1, step (e) comprising calculating the ACE₅₀, which is the titrant concentration at which the signal response of a receptor-ligand pair reaches 50% of its value when the titrant concentration is 0.

9. The method of claim 8, wherein relative K_{d}s of a plurality of ligands are determined such that the ligand with the lowest ACE₅₀ value has the highest K_{d} of the mixture of ligands, and the ligand with the highest ACE₅₀ value has the lowest K_{d} of the mixture of ligands.

10. The method of claim 1, step (e) comprising calculating the K_{di} of a receptor-ligand binding pair, ESᵢ, in the plurality of mixtures by fitting the change in concentration of the receptor-ligand binding pairs, [ESᵢ], in each of the plurality of mixtures as a function of the titrant concentration to the equation of formula (I) or an equation derived from formula (I)
$${k}_{di} = \frac{( {[ E ]}_{0} - {\sum }_{i}^{} [ {ES}_{i} ] ) ( {[ {S}_{i} ]}_{0} - [ {ES}_{i} ] )}{[ {ES}_{i} ]} . \qquad formula ( I )$$

11. The method of claim 10, wherein the relative K_{di}s of a plurality of ligands Sᵢ are determined.

12. The method of claim 1, wherein the initial concentration of receptor, [E]₀, is known and the initial concentration of the ligand, [Sᵢ]₀ is known.

13. The method of claim 1, wherein the concentration of the receptor, [E]₀, is greater than the sum total of the concentration of the ligands, [Sᵢ]₀.

14. The method of claim 1, further determining the whether a ligand Sᵢ binds to the receptor E bind in a competitive manner, an allosteric manner, or a non-competitive manner.

15. The method of claim 14, wherein if a receptor ligand-pair ESᵢ maintains a relatively constant signal response in each of the plurality of mixtures the ligand Sᵢ binds to the receptor E in a non-competitive manner.

16. The method of claim 14, comprising determining the variation in the ratio of signal response of a receptor ligand pair ESᵢ to response of the receptor-titrant pair versus the concentration of the titrant for each of the plurality of mixtures, wherein if the ratios for each of the plurality of mixtures have a linear relationship with the titrant concentration, then the ligand Sᵢ binds to the receptor in a competitive manner, and wherein if the ratios for each of the plurality of mixtures have a non-linear relationship, than the ligand Sᵢ binds to the receptor in an allosteric manner.

17. The method of claim 1, wherein the receptor is a biomolecule.

18. The method of claim 1, wherein the receptor is a polypeptide.

19. The method of claim 1, wherein the receptor is an enzyme.

20. The method of claim 1, wherein the receptor is a nucleic acid.

21. The method of claim 1, wherein the ligand is an organic molecule.

22. The method of claim 1, wherein the ligand is a polypeptide.

23. The method of claim 1, wherein the plurality of mixtures achieves equilibrium of receptor-ligand binding pair, ESᵢ, unbound receptor, and unbound ligand.

24. The method of claim 1, further comprising using liquid chromatography.

25. The method of claim 1, wherein the receptor-bound ligand is separated from each of the plurality of mixtures using size-exclusion-chromatography.

26. The method of claim 1, wherein the receptor-bound ligand is separated from each of the plurality of mixtures using ultrafiltration.

27. The method of claim 1, wherein the signal response is determined using mass spectrometry.

28. The method of claim 1, further comprising disrupting the receptor-ligand binding pair, ES_{i.}

29. The method of claim 1, wherein the signal response of a receptor-ligand binding pair, ESᵢ, is determined by measuring the relative amount of ligand, Sᵢ, in the receptor-ligand binding pair, ESᵢ, in each of the plurality of mixtures.

30. The method of claim 1, wherein the relative amount of ligand, Sᵢ, is determined by evaluating a signal response from a mass spectrometer.

31. A method for determining the equilibrium dissociation constant, K_{d}, of a receptor-ligand binding pair, the method comprising;
(a) providing a mass spectrometer calibrated to the ligand of the receptor-ligand binding pair;
(b) providing a plurality of mixtures, each mixture including a receptor, [E]₀, and a ligand, [S]₀, wherein the concentration of one or more of E₀, and S₀ is chosen such that the binding affinity of S to E can be determined;
(c) allowing each of the plurality of mixtures to reach equilibrium of bound receptor-ligand binding pairs, ES, unbound receptor, and unbound ligand;
(d) separating the receptor-bound ligand from each of the plurality of mixtures;
(e) determining the signal response from the mass spectrometer for the receptor-ligand binding pairs in each of the plurality of mixtures; and
(f) using information known, measured or acquired in steps a-e to fit the concentration of receptor-ligand pair, [ES], and initial, known ligand concentration, [S]₀, to the equation of formula (I)
$${K}_{d} = \frac{( {[ E ]}_{0} - [ ES ] ) ( {[ {S}_{i} ]}_{0} - [ ES ] )}{[ ES ]} \qquad formula ( I )$$
for each of the plurality of mixtures, yielding the K_{d} of the receptor-ligand binding pair.

32. The method of claim 31, wherein each of the plurality of mixtures includes an initial concentration of receptor, [E]₀, and an initial, known concentration of ligand, [S]₀, wherein [E]₀ is about the same in each of the plurality of mixtures and [S]₀ is varied in each of the plurality of mixtures.

33. The method of claim 31, further comprising determining the initial receptor concentration [E]₀ in the mixtures of step (b).

34. The method of claim 31, wherein the receptor is a biomolecule.

35. The method of claim 31, wherein the receptor is a polypeptide

36. The method of claim 31, wherein the receptor is an enzyme.

37. The method of claim 31, wherein the receptor is a nucleic acid.

38. The method of claim 31, wherein the ligand is an organic molecule.

39. The method of claim 31, wherein the ligand is a polypeptide.

40. The method of claim 31, wherein the plurality of mixtures reach equilibrium of bound receptor-ligand binding pairs, unbound receptor, and unbound ligand.

41. The method of claim 31, wherein the receptor-bound ligands are separated from the mixture using size-exclusion-chromatography.

42. The method of claim 31, further comprising using liquid chromatography.

43. The method of claim 31, further comprising disrupting the receptor-ligand binding pairs, ES.

44. The method of claim 31, wherein the concentration of the receptor-ligand binding pair, [ES], is determined in step (e) by measuring the amount of ligand in the receptor-ligand binding pairs, ES, in each of the plurality of mixtures.

45. A method of analyzing the binding kinetics of a receptor-ligand binding pair, the method comprising;
(a) providing a mixture comprising a receptor, [E]₀, and a ligand, [Sᵢ]₀;
(b) allowing the mixture to reach equilibrium of receptor, [E], ligand, [Sᵢ], and receptor-ligand binding pair, [ESᵢ];
(c) treating the mixture with an excess of a competitive inhibitor, I;
(d) measuring a decrease in the receptor-ligand binding pair at a plurality of time points by;
(i) separating the receptor-ligand binding pair from the unbound ligand; and
(ii) determining a signal response of the receptor-ligand binding pair for each of the plurality of time points with an analytical device; and
(e) using the information known, measured, or acquired from steps (a)-(d) to evaluate the binding kinetics of the receptor-ligand binding pair.

46. The method of claim 45, wherein the signal response of the receptor-ligand binding pair is measured with an analytical device.

47. The method of claim 45, wherein the mixture of step (a) comprises a plurality of ligands, Sᵢ.

48. The method of claim 45, wherein at least 90% of the plurality of ligands, Sᵢ, have a unique molecular mass.

49. The method of claim 45, wherein the binding kinetics are evaluated using the information known, measured, or acquired from steps (a)-(d) to calculate the dissociation rate, kₛ₂ of the receptor-ligand binding pair by fitting the change in signal response of the receptor-ligand binding pair over time to the equation of formula (XVIII) or a derivative thereof
[ES]=[ES]ₜ₌₀e^{-ks2·t} formula (XVIII).

50. The method of claim 45, comprising identifying a ligand that binds in a non-competitive manner wherein if the a ligand-receptor binding pair maintains a relatively constant concentration at each of the plurality of time points, than the ligand is binding to the receptor in a non-competitive manner.

51. The method of claim 45, wherein the binding kinetics of at least two of the plurality of ligands, Sᵢ, are compared.

52. The method of claim 45, wherein the receptor is a biomolecule.

53. The method of claim 45, wherein the receptor is a polypeptide.

54. The method of claim 45, wherein the receptor is an enzyme.

55. The method of claim 45, wherein the receptor is a nucleic acid.

56. The method of claim 45, wherein the ligand is an organic molecule.

57. The method of claim 45, wherein the ligand is a polypeptide.

58. The method of claim 45, wherein the competitive inhibitor is an organic molecule.

59. The method of claim 45, wherein the competitive inhibitor is a polypeptide.

60. The method of claim 45, further comprising subjecting the receptor-bound ligand to liquid chromatography.

61. The method of claim 45, wherein the receptor-bound ligand is separated from the unbound ligand using size-exclusion-chromatography.

62. The method of claim 45, wherein the signal response is determined using mass spectrometry.

63. The method of claim 45, further comprising disrupting the receptor-ligand binding pair.

64. The method of claim 45, wherein the signal response of the receptor-ligand binding pair is determined by measuring the relative amount of ligand in the receptor-ligand binding pair.

65. The method of claim 45, further comprising determining the half-life, t_{1/2}, of the receptor ligand binding pair.
